(19) United States
(12) Patent Application Publication
(10) Pub. No.: US 2021/0220258 A1
(43) Pub. Date: Jul. 22, 2021

(54) ORAL DOSAGE FORM WITH SURFACE DELIVERY OF ACTIVE AGENT

(71) Applicant: Entrega Inc., Boston, MA (US)

(72) Inventors: Daniel BONNER, Sharon, MA (US); John JANTZ, Arlington, MA (US); Thomas H. JOZEFIAK, Belmont, MA (US); Bhushan PATTNI, Canton, MA (US)

(21) Appl. No.: 17/220,456

(22) Filed: Apr. 1, 2021

Related U.S. Application Data
(63) Continuation of application No. PCT/US2019/054419, filed on Oct. 3, 2019.
(60) Provisional application No. 62/741,790, filed on Oct. 5, 2018.

(51) Int. Cl. A61K 9/00; A61K 9/06; A61K 45/06; A61M 31/00; A61K 9/48; A61K 47/36; A61K 47/32; A61K 47/34

(52) U.S. Cl. CPC A61K 9/006; A61K 9/06; A61K 45/06; A61K 47/34; A61K 9/4891; A61K 47/36; A61K 47/32; A61M 31/002

(57) ABSTRACT

An oral dosage form provides a delivery structure having active agent delivery regions at an exterior surface of a body of super-porous hydrogel material, and a protective coating, for delivery of the active agent to an intestinal site.

The present application claims priority as a continuation of PCT/US2019/054419, filed on Oct. 3, 2019, which claims priority to provisional application 62/741,790 filed on Oct. 5, 2018, each of which is hereby incorporated by reference in their entireties herein.

Oral dosing of active agents is attractive for many reasons, including ease of administration and high patient compliance. However, for some active agents, such as poorly absorbed, sensitive (i.e., pH sensitive, enzyme-sensitive, and the like), and/or high molecular weight active agents, oral dosing may be less effective or ineffective for achieving sufficient blood concentration of the active agent as compared to alternative dosing strategies. For example, active agents such as proteins and other macromolecules may be enzymatically degraded in the gastrointestinal tract and/or may have limited transport across the intestinal epithelium.

One potential strategy for circumventing the hostile environment of the gastrointestinal tract is to alter the environment through the use of protease inhibitors and/or derivatization of agents with polyethylene glycol to prevent enzymatic degradation. Another potential strategy is to increase the permeability of the tissue in the gastrointestinal tract such that absorption of an agent increases. An agent may be formulated with an excipient that can, for example, open the tight junctions of the intestine to allow an agent to pass through the intestinal epithelium. A further approach to improving delivery of an agent in the gastrointestinal tract is to apply an enteric coating to the agent such that the agent is not exposed to the harsh pH conditions of the stomach, and is instead released in the small intestine, where absorption occurs more readily.

Another technique for drug delivery is the use of superporous hydrogels (SPHs) as a part of a drug delivery system. SPHs may swell in a gastric medium, and as such may be retained in the gastric environment, thereby increasing the time an orally administered drug resides, e.g., in the gastric fluid of the stomach and/or upper GI tract (see, e.g., U.S. Pat. No. 7,988,992 to Omidian et al.; Recent Developments in Superporous Hydrogels, Journal of Pharmacy and Pharmacology, Omidian et al., 59:317-327 (2007); U.S. Pat. No. 6,271,278 to Park et al.). Drug delivery systems have also been described that use a “shuttle” made of SPH and/or superporous hydrogel composite (SPHC), containing a core that is embedded into the SPH and/or SPHC body having the active ingredient (see, e.g., Development and Characterization of a Novel Peroral Peptide Drug Delivery System, J. Controlled Release, Dorkoosh et al., 71:307-318 (2001).

However, a need remains for drug delivery systems that are capable of providing improved delivery of an agent to the gastrointestinal tract, such as in a form that allows the active agent to be readily absorbed by the intestinal tissue, without excessive degradation thereof. A need also remains for drug delivery systems and/or SPH compositions that are capable of providing improved active agent delivery to the intestinal tract.

According to one embodiment herein, a pharmaceutically acceptable oral dosage form for delivery of an active agent to an intestinal site is provided. The dosage form includes a delivery structure having a monolithic body of super porous hydrogel (SPH) material, the monolithic body having an exterior surface, and one or more active agent delivery regions to deliver the active agent, the one or more active agent delivery regions being located at the exterior surface of the monolithic body, and a protective coating covering at least a portion of the delivery structure, wherein at least 10 wt % of the active agent contained in the oral dosage form is located in the one or more active agent delivery regions at the exterior surface of the monolithic body.

According to another embodiment, a method of forming a super-porous hydrogel (SPH) material is provided, the method comprising forming a polymerization mixture by combining (i) a structural support material comprising at least one ionically charged structural support polymer having a molecular weight of at least 50,000 g/mol, the ionically charged structural support polymer having a plurality of ionically charged chemical groups, (ii) a monomer material comprising at least one ionically charged ethylenically-unsaturated monomer, and (iii) at least one cross-linking agent, forming a foam of the polymerization mixture, and polymerizing the foam to form a porous crosslinked polymeric structure having ion-pairing between a cross-linked polymer matrix formed by polymerization of the ionically charged ethylenically-unsaturated monomer with the cross-linking agent, and the ionically charged structural support polymer, wherein each of the ionically charged chemical groups of the ionically charged structural support polymer each have an ionic charge that is the opposite of that of a charge of the ionically charged ethylenically-unsaturated monomer.

According to yet another embodiment, a super-porous hydrogel (SPH) material for the SPH body is provided, comprising a porous cross-linked polymeric structure comprising a crosslinked polymer matrix having a repeat structure of monomers comprising ionically charged chemical groups, about an ionically charged structural support polymer comprising ionically charged chemical groups, the ionically charged structural support polymer having a molecular weight of at least 50,000 g/mol, wherein at least some of the ionically charged groups of the crosslinked polymer matrix are ion-paired with the ionically charged groups of the ionically charged structural support polymer, and wherein each of the ionically charged chemical groups of the ionically charged structural support polymer each have an ionic charge that is the opposite of that of a charge of the ionically charged chemical groups of the repeat structure of the cross-linked polymer matrix.

According to yet another embodiment, a method of forming a super-porous hydrogel (SPH) material comprises forming a polymerization mixture by combining (i) a monomer material comprising at least one cationically charged ethylenically-unsaturated monomer, and optionally at least one non-ionically charged ethylenically unsaturated monomer, and (ii) at least one cross-linking agent, forming a foam of the polymerization mixture, and polymerizing the foam to form a porous crosslinked polymeric structure formed by polymerization of the cationically charged ethylenically-unsaturated monomer with the cross-linking agent, and optionally with the neutral ethylenically unsaturated monomer, wherein the porous crosslinked polymeric structure comprises a Maximum Swell Ratio of at least 20, and a Compressive Strength as measured by the Yield Point of at least 5000 Pascals.

According to yet another embodiment, a super-porous hydrogel (SPH) material, comprises a porous cross-linked polymeric structure comprising a crosslinked polymer matrix having a repeat structure of monomer residues obtained from cationically charged ethylenically-unsaturated monomers, and optionally monomer residues obtained from non-ionically charged ethylenically-unsaturated monomers, wherein the porous cross-linked polymeric structure comprises a Maximum Swell Ratio of at least 20, and a Compressive Strength as measured by the Yield Point of at least 5000 Pascals.

### BRIEF DESCRIPTION OF THE FIGURES

FIG. 1 shows one embodiment of a pharmaceutically acceptable oral dosage form for delivery of an active agent to an intestinal site, having particles and/or granules containing active agent at an exterior surface of a body of SPH, according to aspects of the present disclosure.

FIG. 2 shows another embodiment of a pharmaceutically acceptable

oral dosage form for delivery of an active agent to an intestinal site, having one or more compressed tablets containing active agent attached to an exterior surface of a body of SPH, according to aspects of the present disclosure.

FIG. 3 shows yet another embodiment of a pharmaceutically acceptable oral dosage form for delivery of an active agent to an intestinal site, having a coating containing active agent on an exterior surface of a body of SPH, according to aspects of the present disclosure.

FIG. 4 shows yet another embodiment of a pharmaceutically acceptable oral dosage form for delivery of an active agent to an intestinal site, having a biodegradable film containing active agent on an exterior surface of a body of SPH, according to aspects of the present disclosure.

FIG. 5 shows another embodiment of a pharmaceutically acceptable oral dosage form for delivery of an active agent to an intestinal site, having a lipid composition containing active agent at an exterior surface of a body of SPH, according to aspects of the present disclosure.

FIG. 6 shows yet another embodiment of a pharmaceutically acceptable oral dosage form for delivery of an active agent to an intestinal site, having active agent permeating into an exterior surface of a body of SPH, according to aspects of the present disclosure.

FIGS. 7 and 8 show different embodiments of a pharmaceutically acceptable oral dosage form for delivery of an active agent to an intestinal site, having different shapes of SPH body provided in the form, according to aspects of the present disclosure.

FIGS. 9A-9D show different embodiments of a pharmaceutically acceptable oral dosage form for delivery of an active agent to an intestinal site, having a biodegradable film containing active agent on an exterior surface of a body of SPH, according to aspects of the present disclosure.

FIG. 10A is a plot of the Swell Ratio over time for an embodiment of an SPH material, according to aspects of the present disclosure.

FIG. 10B is a plot of Swell Ratio Percentage over time for an embodiment of an SPH material, according to aspects of the present disclosure.

FIG. 10C is a plot of the stress versus strain measurement for an embodiment of an SPH material, according to aspects of the present disclosure.

FIG. 10D is a plot of the force versus strain measurement for an embodiment of an SPH material, according to aspects of the present disclosure.

FIG. 10E is a plot of the force exerted over time for an embodiment of an SPH material, according to aspects of the present disclosure.

FIG. 10F is a plot of Swell Ratio over time for an embodiment of an SPH material, according to aspects of the present disclosure.

FIG. 10G is a plot of Swell Ratio Percentage over time for an embodiment of an SPH material, according to aspects of the present disclosure.

FIG. 10H is a plot of the stress versus strain measurement for an embodiment of an SPH material, according to aspects of the present disclosure.

FIG. 10I is a plot of the force exerted over time for an embodiment of an SPH material, according to aspects of the present disclosure.

FIG. 11A is a plot of Swell Ratio over time for an embodiment of an SPH material, according to aspects of the present disclosure.

FIG. 11B is a plot of Swell Ratio Percentage over time for an embodiment of an SPH material, according to aspects of the present disclosure.

FIG. 11C is a plot of the stress versus strain measurement for an embodiment of an SPH material, according to aspects of the present disclosure.

FIG. 11D is a plot of the force versus strain measurement for an embodiment of an SPH material, according to aspects of the present disclosure.

FIG. 11E is a plot of the force exerted over time for an embodiment of an SPH material, according to aspects of the present disclosure.

FIG. 12 is a plot of the stress versus strain measurement for an embodiment of a comparative SPH material, according to aspects of the present disclosure.

FIGS. 13A-13C are plots of Swell Ratios over time for embodiments of SPH material, according to aspects of the present disclosure.

FIG. 13D is a bar graph showing Maximum Swell Ratios for different compositions of SPH material, according to aspects of the present disclosure.

FIG. 13E is a bar graph showing Swell Ratio Percentage at one minute for different compositions of SPH material, according to aspects of the present disclosure.

FIGS. 13F-13H are plots of the stress versus strain measurement for different compositions of SPH material, according to aspects of the present disclosure.

FIG. 131 is a bar graph showing yield point for different compositions of SPH material, according to aspects of the present disclosure.

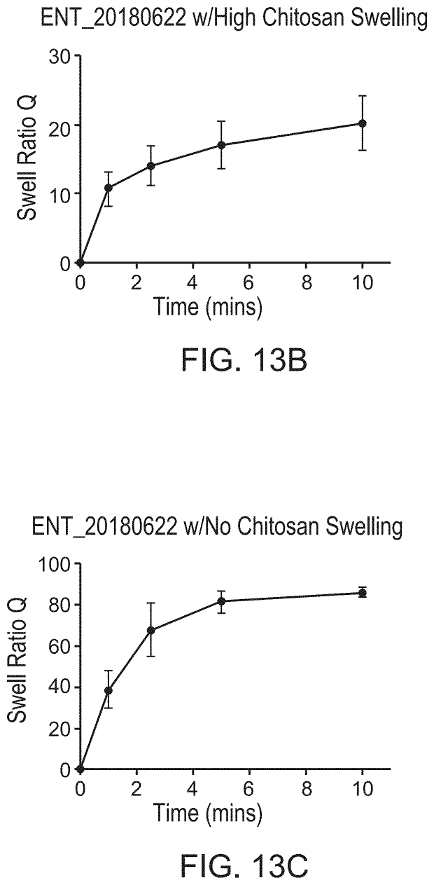

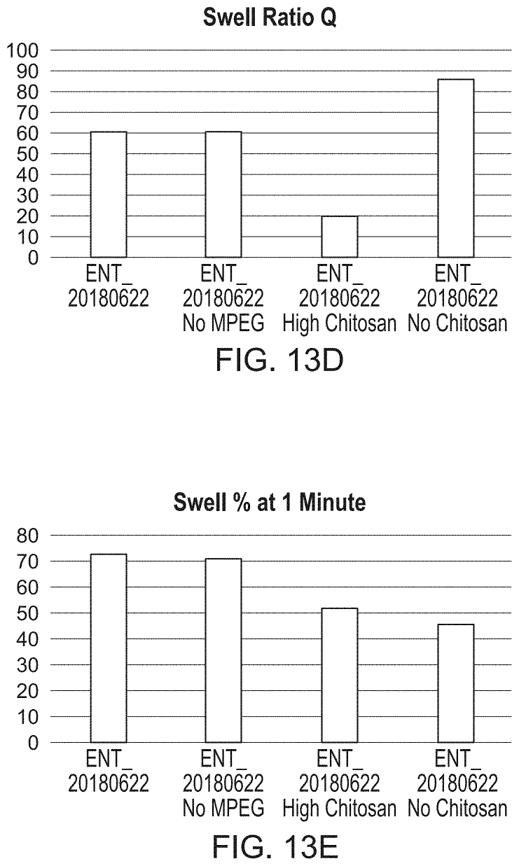

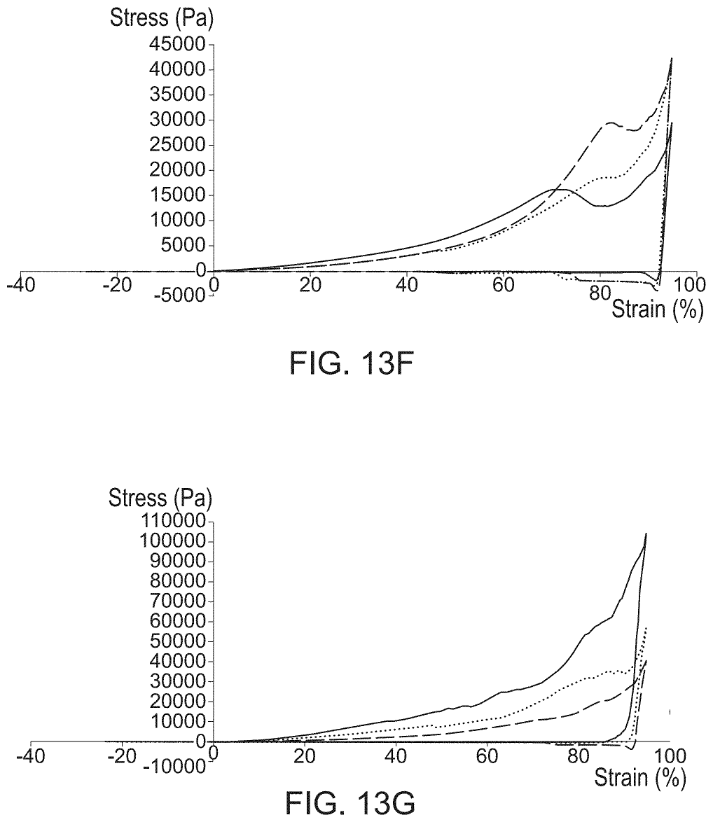

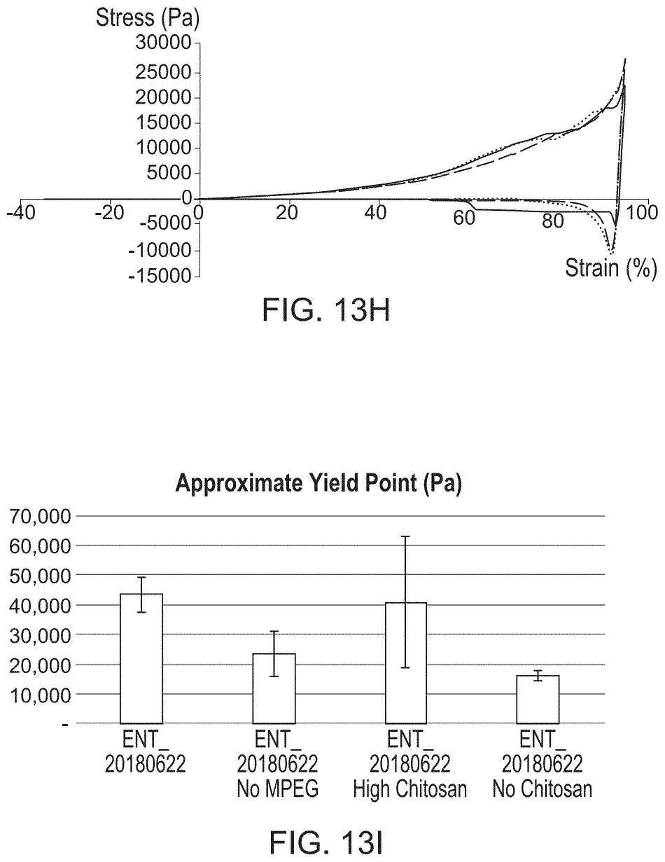

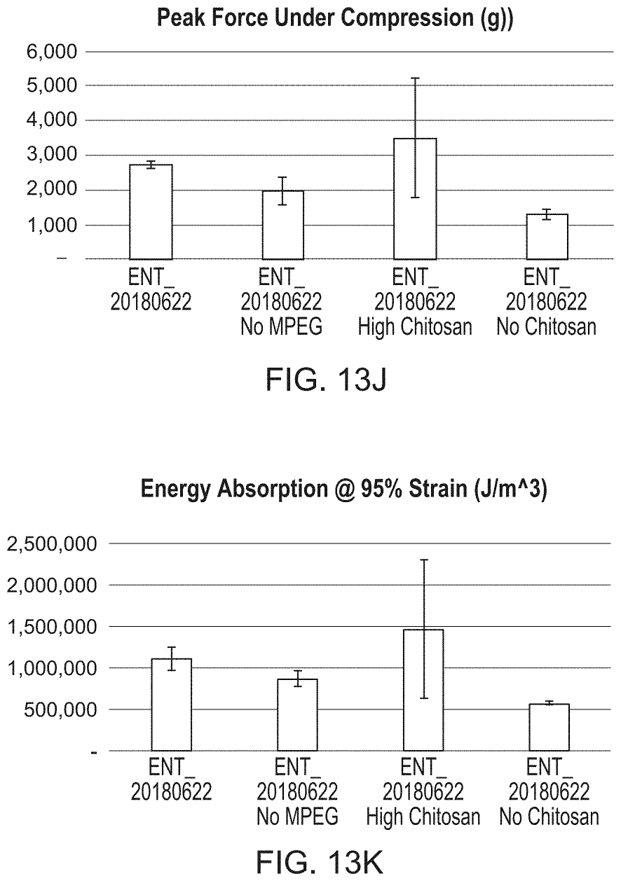

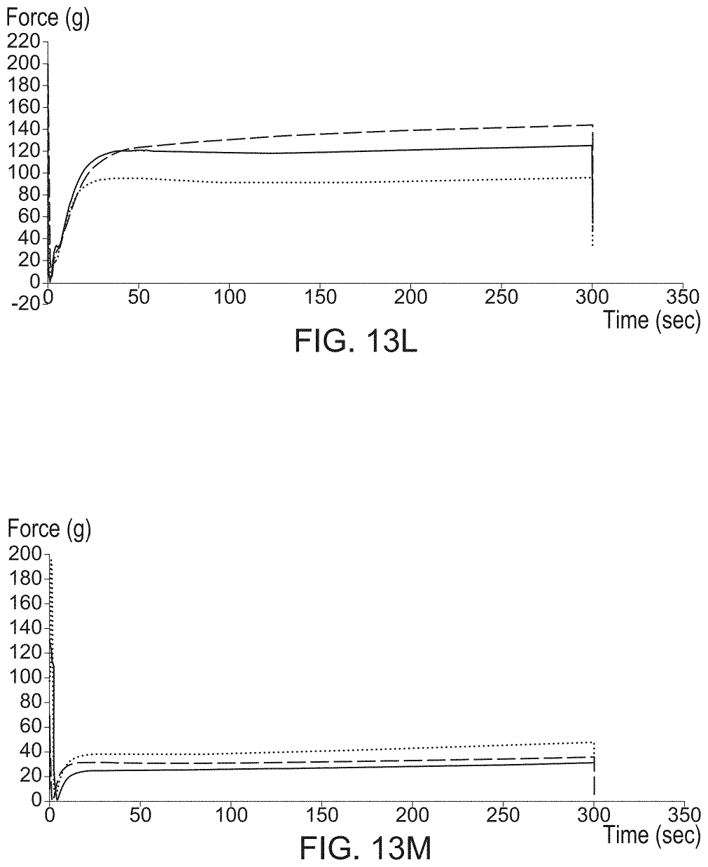

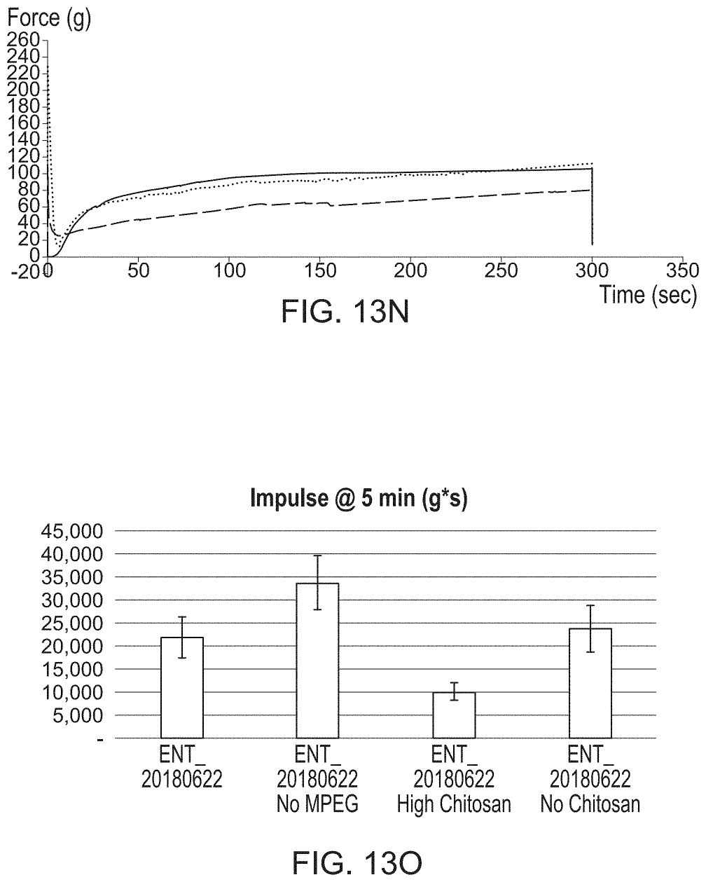

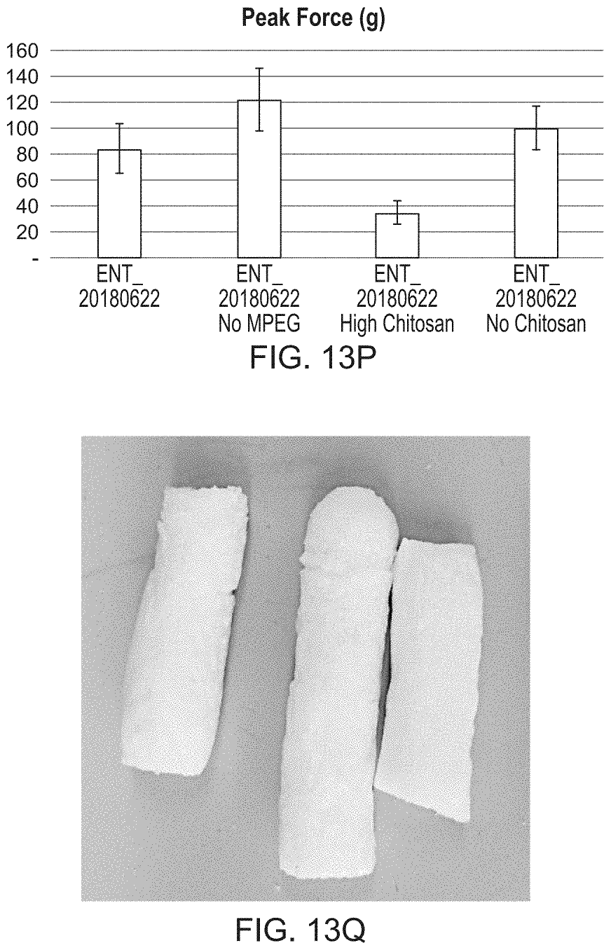

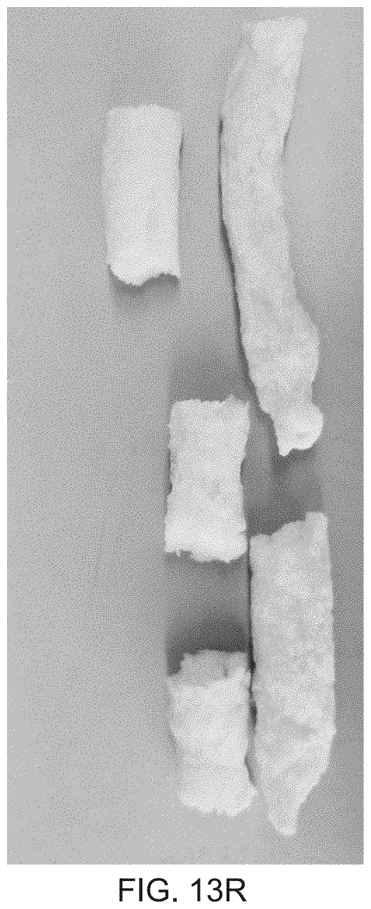

FIG. 13J is a bar graph showing peak force under compression for different compositions of SPH material, according to aspects of the present disclosure.

FIG. 13K is a bar graph showing energy absorption at 95% strain for different compositions of SPH material, according to aspects of the present disclosure.

FIGS. 13L-13N are plots of the force exerted over time for different compositions of SPH material, according to aspects of the present disclosure.

FIG. 13O is a bar graph showing impulse at 5 minutes for different compositions of SPH material, according to aspects of the present disclosure.

FIG. 13P is a bar graph showing peak force for different compositions of SPH material, according to aspects of the present disclosure.

FIGS. 13Q-13R are images of SPH bodies with different compositions of SPH material, according to aspects of the present disclosure.

Other aspects, embodiments and features of the inventive subject matter will become apparent from the following detailed description when considered in conjunction with the accompanying drawing. The accompanying figures are schematic and are not intended to be drawn to scale. For purposes of clarity, not every element or component is labeled in every figure, nor is every element or component of each embodiment of the inventive subject matter shown where illustration is not necessary to allow those of ordinary skill in the art to understand the inventive subject matter.

### Definitions

“Agent” as used herein refers to any treatment agent that can be administered to a patient for treatment and/or prevention of a disease and/or condition, including but not limited to a pharmaceutical agent, a drug, a small molecule drug, a drug conjugate, a prodrug, an antibody or an antibody fragment, a nucleic acid, a protein, a peptide, a polysaccharide, a small organic molecule (e.g., with a molecular weight of about 500 Da or less), a metabolically activated agent (e.g., a metabolite), a nutrient, a supplement, and the like, unless specified otherwise.

“Biodegradable” as used herein refers to materials that, when introduced into the body of an individual, patient, or subject, are broken down by cellular machinery, chemical processes (e.g., hydrolysis), or physical processes (e.g., dissolution) into components (sometimes referred to as “degradation products”) that the body can either reuse or dispose of without significant toxic effect. In some instances, the degradation products may also be biocompatible.

“Monolithic” as used with respect to the body of SPH material herein refers to a body that is formed of a single piece of SPH material, as opposed to multiple individual SPH particles or fragments. For example, the monolithic body of SPH material may be a body of material that is formed via polymerization of monomers in a foam optionally together with cross-linking agents and/or structural support polymers, to form the SPH material. According to some embodiments, a monolithic body of SPH may break up into multiple smaller pieces following administration to a patient, such as for example as caused by peristaltic forces in the gastrointestinal tract.

“Mucoadhesive” as used herein refers to a composition having the capacity to bind to a mucosal surface.

“Superporous Hydrogel (SPH)” as used herein refers to porous hydrophilic crosslinked polymeric structures that are capable of absorbing fluids. In certain embodiments, a superporous hydrogel (SPH) material may have pore sizes of at least 0.5 microns to at least 10 microns, such as up to 80 microns, or even 200 microns or larger, although the pore size is typically less than about 1 mm. However, SPH materials may also come in a variety of different pore sizes, pore distributions, pore shapes, etc., and so are not limited to any one particular pore size and/or distribution. Furthermore, unless specified otherwise herein, the term “Superporous Hydrogel” or “SPH” is intended to encompass different forms of superporous hydrogels including simple or first generation SPHs (CSPHs), SPH composites (SPHCs), and SPH hybrids (SPHHs), for example as described in “Recent Developments in Superporous Hydrogels” by Omidian et al. (J. of Pharmacy and Pharmacology, 59: 317-327 (2007)).

“Dried SPH” or SPH in a “Dried State” as used herein refers to SPH material having a water content that is the same as that for SPH material that has been dried for at least 18 hours in a convection oven set to 150° F. at standard pressure.

“Compressible SPH” or SPH in a “Compressible State” refers to SPH material that has absorbed fluid and/or moisture as compared to the Dried State, up to a point of no more than 10% mass gain of fluid from the Dried State, as measured at approximately standard temperature and pressure.

“Hydrated SPH” or SPH in a “Hydrated State” refers to SPH material that has absorbed an amount of fluid and/or moisture that is increased over that of the SPH material in the “Compressible State,” corresponding to more than 10% mass gain of fluid as compared to the SPH material in the Dried State.

“Compressed SPH” or SPH in a “Compressed State” refers to a sample of SPH material that has been compressed by applying compressive forces to the SPH sample to reduce the volume of the SPH sample as compared to an Uncompressed State where no compressive forces have been applied. For example, in some embodiments, the Compressed SPH may have a Compressed Volume that is less than 85%, less than 75%, less than 60% and/or less than 50% of an Uncompressed Volume of the same SPH material in an Uncompressed State, as measured by external dimensions of the sample of SPH material. In some embodiments, the Compressed SPH may be maintained at the Compressed Volume by continuous application of compressive forces thereto, or in other embodiments the Compressed SPH may be maintained at the Compressed Volume even upon cessation of application of compressive forces thereto. In certain embodiments, the Compressed SPH is prepared by using SPH material having moisture absorbed therein corresponding to the Compressible State, and compressing the volume of the sample of SPH material in the Compressible State to the Compressed Volume. In other embodiments, the Compressed SPH may be prepared from SPH in the Dried State. The Compressed and Uncompressed Volumes of the SPH sample correspond to the effective volume of the SPH sample as measured using the external dimensions of the SPH sample. For example, for an SPH sample having a cylindrical shape, the effective volume would be calculated using the formula: V_{Eff}=π×(½×Diameter)²×Length, and as another example, for an SPH sample having a rectangular prism shape, the effective volume would be calculated using the formula: V_{Eff}=Length×Width×Height.

In some embodiments, the dimensions such as the length, diameter, width, height, etc., may be determined by using calipers as described below, or may be determined by another method as understood by those of ordinary skill in the art. Also, in certain embodiments, for irregularly shaped samples, the sample may be cut to a more regular shape to allow for ready determination of dimensions.

“Uncompressed SPH” or SPH in an “Uncompressed State” refers to SPH material in a state where substantially no compressive forces are being exerted on the SPH material, other than ambient pressure at approximately standard atmospheric pressure. For purposes of clarity, the SPH material described herein is assumed to be in the Uncompressed State, unless expressly indicated otherwise.

“Effective Density” refers to the density of a sample of SPH material in its Dried State, as determined from the mass of the SPH material divided by the sample effective volume. Specifically, the sample effective volume is that as measured by the external dimensions of the SPH sample. For example, for an SPH sample having a cylindrical shape, the effective volume would be calculated using the formula: V_{Eff}=π×(½×Diameter)²×Length, and as another example, for an SPH sample having a rectangular prism shape, the effective volume would be calculated using the formula: V_{Eff}=Length×Width×Height. The dimensions such as the length, diameter, width, height, etc., may be determined by using calipers as described below, or may be determined by another method as understood by those of ordinary skill in the art. Also, for irregularly shaped samples, the sample may be cut to a more regular shape to allow for ready determination of dimensions. The Effective Density for an SPH sample is determined as follows (performed at approximately standard temperatures and pressures):

a. cut an approximately 500 mg piece of Dried SPH sample and record actual mass (Mass);

b. measure the external dimensions of the SPH sample, such as with calipers, and calculate the effective volume V_{Eff} using the external dimensions;

c. use the following formula to calculate the effective density:

Effective Density (g/cm³)=Mass (g)/V_{Eff} (cm³).

In certain embodiments, the Effective Density may be that measured for the SPH sample in an Uncompressed State. In other embodiments, the Effective Density may be that measured for the SPH sample in a Compressed State.

“Swell Ratio” as used herein is a measure of the mass of fluid taken up by a sample of SPH at a point in time following introduction of the fluid to the SPH sample, divided by the initial mass of the SPH sample. The Swell Ratio can be expressed as follows: Q (Swell Ratio)=(Swollen Mass-Initial Mass)/Initial Mass. The method used to determine the Swell Ratio for a mass of SPH, such as an SPH body, is as follows (performed at approximately standard temperature and pressure):

a. cut an approximately 300 to 500 mg piece of dried SPH sample and record actual mass (Initial Mass);

b. record mass of a container (Container Mass);

c. place 300 mL of deionized water in the container;

d. place dried SPH sample in the container with the deionized water, and start timer;

e. at a selected time interval, such as at 1 minute, 2.5 minutes, 5 minutes, and/or 10 minutes, stop the timer, drain the fluid from the container, and weigh the container with SPH sample (weight of SPH sample at selected time interval−Container Mass=Swollen Mass);

f. to obtain Swell Ratio at later time intervals, replace the deionized water and re-start timer, and repeat step (e).

g. calculate Swell Ratio Q at one or more of the selected time intervals as follows:

Q=(Swollen Mass-Initial Mass)/Initial Mass.

For example, for an SPH sample having an Initial Mass of 0.2 grams that swells to 13 grams total after introduction of the deionized water, a Swell Ratio for the SPH sample may be calculated as (13 grams-0.2 grams)/0.2 grams=64.

“Maximum Swell Ratio” as used herein refers to the Swell Ratio of a sample of SPH as determined at a time interval of 10 minutes following introduction of the fluid to the SPH sample.

“Swell Ratio Percentage” as used herein refers to the percentage of the Maximum Swell Ratio that a Swell Ratio corresponds to as measured at a select time interval. For example, for a Maximum Swell ratio of 100 for a SPH sample, a Swell Ratio of 50 as measured at a time interval of 1 minute would correspond to a Swell Ratio Percentage of 50%.

“Swelling Speed” as used herein refers to the speed with which an SPH sample reaches a predetermined Swell Ratio Percentage. For example, an SPH sample may have a Swelling Speed such that it reaches a Swell Ratio Percentage of at least 30% in 1 minute, a Swell Ratio Percentage of 50% in 2 minutes, and a Swell Ratio Percentage of 100% in 10 minutes.

“Compressive Strength” as used herein refers to the compressive force required to “break” a sample of Hydrated SPH, as determined by onset of a discontinuous change in the stress versus strain relationship with application of increasing compressive force. The Compressive Strength may be measured with a texture analyzer, such as a TA.XT Plus Connect Texture Analyzer available from Texture Technologies Corp., although other similar texture analyzers may also be used to obtain measures of the Compressive Strength, as would be understood to those of ordinary skill in the art. The Compressive Strength may in some embodiments be reported as the Yield Point, which is the maximum stress measured in units of Pa that is attained before the SPH sample “breaks” and the stress drops (i.e., before the slope of the stress as plotted versus the strain becomes negative). The Compressive Strength may also in some embodiments be reported as the Peak Force Under Compression, which corresponds to the maximum force applied to the SPH sample in units of grams at a point of 95% compressive strain of the SPH sample. The Compressive Strength may further in some embodiments be reported as the Energy Absorbed by the SPH sample, which corresponds to the energy absorbed in units of J/m³ by the sample of SPH when loaded to 95% compressive strain (the area under the curve of the stress versus strain graph). The Compressive Strength, as reported in terms of the Yield Point, Peak Force, and/or Energy Absorbed, is measured as follows (performed at approximately standard temperature and pressure):

a. cut an approximately 300 to 500 mg piece of dried SPH sample, and record mass;

b. place the SPH sample in deionized water and allow it to swell to equilibrium for 10 minutes;

c. place the SPH sample in a container on the testing platform of texture analyzer;

d. calibrate the probe “0 height” to the bottom of the container;

e. check that 1 inch diameter circular testing probe is attached, and set units to Pascals (y-axis) and % strain (x-axis);

f. place the swollen hydrogel in the container, centered under the probe, and lower testing probe to approximately 0.5 inches above the SPH sample.

g. begin test, increase load on the hydrogel up to 95% strain by lowering the probe at a rate of 2 mm/s;

h. determine Compressive Strength values, including any of the Peak Force Under Compression, Energy Absorption up to 95% strain, and Yield Point.

“Individual,” “patient,” or “subject” as used herein are used interchangeably and refer to any animal, including mammals, preferably mice, rats, guinea pigs, and other rodents; rabbits; dogs; cats; swine; cattle; sheep; horses; birds; reptiles; or primates, such as humans.

“Radial Force” as used herein refers to the maximum outward force exerted by a SPH sample as it swells with uptake of a fluid. The Radial Force may be measured with a texture analyzer, such a TA.XT Plus Connect Texture Analyzer available from Texture Technologies Corp., although other similar texture analyzers may also be used to obtain measures of the Radial Force, as would be understood to those of ordinary skill in the art, and may be measured in units of grams of force. The Radial Force is determined as follows (performed at approximately standard temperature and pressure):

a. cut an approximately 300 to 500 mg piece of dried SPH sample, and record mass;

b. place SPH sample in container on testing platform of TA Plus texture analyzer;

c. check that 1″ diameter testing probe is attached, lower testing probe to approximately 0.5 inches above the SPH sample, and fill serological pipette with 25 mL deionized water;

d. contact probe to SPH sample on a surface of the SPH sample where the Radial Force is to be measured, such as a surface that is parallel to the longitudinal axis for an elongated SPH body, and add the 25 mL of water to container (about 1 second after contacting the SPH sample with the probe);

e. over the course of about 5 minutes, measure the force exerted onto the stationary probe by the swelling SPH sample, as a function of time;

f. determine the Radial Force as the maximum force exerted by the contacted surface at any time during the 5 minutes, and optionally an Impulse value corresponding to the area under the curve of the plot of the force exerted as a function of time.

“Volume Swell Ratio” as used herein is a measure of the change in volume of an SPH sample following uptake of fluid by the SPH sample, divided by the initial volume of the SPH sample. The Volume Swell Ratio can be expressed as follows: Volume Swell Ratio=(Final Volume (cm³)-Initial Volume (cm³))/Initial Volume(cm³). Specifically, the volumes (Final Volume and/or Initial Volume) are those as measured by the external dimensions of the SPH sample. For example, for an SPH sample having a cylindrical shape, the volume would be calculated using the formula: V_{Eff}=π×(½×Diameter)²×Length, and as another example, for an SPH sample having a rectangular prism shape, the effective volume would be calculated using the formula: V_{Eff}=Length×Width×Height. The dimensions such as the length, diameter, width, height, etc., may be determined by using calipers as described below, or may be determined by another method as understood by those of ordinary skill in the art. Also, for irregularly shaped samples, the sample may be cut to a more regular shape to allow for ready determination of dimensions. The method used to determine the Volume Swell Ratio for a mass of SPH, such as an SPH body, is as follows (performed at approximately standard temperature and pressure):

a. cut an approximately 500 mg piece of SPH sample;

b. measure the external dimensions of the SPH sample, such as with calipers, and calculate the volume (Initial Volume);

c. place the SPH sample in 300 mL of deionized water in a container and wait 10 minutes for the SPH sample to reach equilibrium;

d. after 10 minutes, remove the SPH sample from the water and measure the external dimensions, such as with calipers, and calculate the volume (Final Volume);

e. use the following formula to calculate the Volume Swell Ratio:

V=(Final Volume (cm³)−Initial Volume(cm³))/Initial Volume(cm³).

Furthermore, according to certain embodiments, the SPH sample may be compressed to a compressed volume that corresponds to the Initial Volume, prior to contacting the SPH sample with deionized water in the container, in which case the Volume Swell Ratio is a measure of the extent of swelling from a compressed state. In certain embodiments, the Volume Swell Ratio may be that for an SPH sample with an Initial Volume as measured in an Uncompressed State. In other embodiments, the Volume Swell Ratio may be that for an SPH sample with an Initial Volume as measured in a Compressed State.

“Pharmaceutically acceptable carrier” or “pharmaceutically acceptable excipient” as used herein refers to any and all solvents, dispersion media, coatings, isotonic and absorption delaying agents, and the like, that are biocompatible and otherwise suitable for administration to an Individual.

“Pharmaceutical composition” as used herein refers to a composition comprising at least one agent as disclosed herein formulated together with one or more pharmaceutically acceptable carriers and/or excipients.

“Pharmaceutically or pharmacologically acceptable” as used herein refers to molecular entities and compositions that are acceptable for administration to an animal, or a human, as appropriate, for example in not producing an excessive adverse, allergic, or other untoward reaction.

“Capsule Escape Assay” as used herein refers to an assay for determining the amount of time required for an SPH sample to fully expand and escape a capsule into which it has been placed. The Capsule Escape Assay is performed as follows (performed at approximately standard temperature and pressure, except where specified):

a. cut piece of dried SPH sample sized to fit within a 000 HPMC capsule;

b. place the SPH sample inside the 000 HPMC capsule;

c. place 20 mL of approximately 37° C. deionized water inside a 40 mL glass vial;

d. place the 000 HPMC capsule containing the SPH sample inside the vial and orient it horizontally;

e. gently rotate the vial along its long axis at a rate of no more than 60 rpm to promote fluid mixing;

f. start timer and cease rotation upon first visual indication that the capsule is cracking/rupturing;

g. continue to record time until SPH sample is fully expanded and is free of the capsule;

h. the time until the SPH sample is fully expanded, from the first visual indication that the capsule is cracking/rupturing, is the Capsule Escape Time for the SPH sample, and other expansion mechanics may also be recorded during the Capsule Escape Test.

“Treating” as used herein refers to any effect, for example, lessening, reducing or modulating, that results in the improvement of the condition, disease, disorder, and the like.

The singular forms “a,” “an,” and “the,” as used herein, include plural referents unless the context clearly dictates otherwise.

The terms “comprising,” “comprises,” “including,” and “includes” should be interpreted as referring to elements, components, or steps in a non-exclusive manner, indicating that the referenced elements, components, or steps may be present, utilized, or combined with other elements, components, or steps that are not expressly referenced.

### DETAILED DESCRIPTION

Aspects of the present disclosure are directed to dosage forms, systems and methods for the oral, trans-intestinal, and/or trans-mucosal delivery of an active agent. In particular, aspects of the present disclosure relate to an oral dosage form having a delivery structure that comprises a monolithic body of superporous hydrogel (SPH) with an exterior surface, and one or more active agent delivery regions on the exterior surface, with a protective coating covering at least a portion of the delivery structure, which provides for delivery of the active agent from the exterior surface of the monolithic body. Aspects of the present disclosure also relate to a SPH material that may be suitable for the dosage forms, such as for use as the SPH body having the exterior surface for the delivery of the active agent. Aspects of the present disclosure further relate to dosage forms comprising SPH in a form with physical properties that allow for excellent active agent delivery characteristics at an intestinal site, such as swelling ratio, swelling speed, compressive strength and radial strength. Further aspects of the present disclosure provide for methods of manufacturing SPH and/or dosage forms with delivery structures containing SPH, as well as methods of administering active agents with the dosage forms and/or SPH.

Without being limited to any theory, it is believed that aspects of the dosage form herein may be capable of providing for enhanced bioavailability of active agents delivered via the dosage form. For example, the monolithic body of SPH may provide a highly swellable body that is capable of rapidly expanding at an intestinal site, such that at least a portion of the exterior surface of the SPH monolithic body is pressed into contact with neighboring intestinal tissue at the intestinal site. By contacting the exterior surface of the SPH monolithic body with the intestinal tissue, the active agent at the exterior surface may be physically contacted with the intestinal tissue and/or placed in close proximity with the intestinal tissue, thereby allowing the intestinal tissue to more readily absorb the active agent to provide enhanced bioavailability of the active agent. The SPH monolithic body may even, according to certain aspects, possess a sufficient radial strength to press the active agent on the exterior surface against the intestinal tissue, thereby increasing absorption by the intestinal tissue. According to certain aspects, the SPH monolithic body may even be capable of swelling to a sufficient extent, and with properties such as a sufficient radial strength and/or compressive strength, such that the monolithic SPH body may be retained at the intestinal site in a sufficiently intact form to provide for delivery of the active agent, such as for example, according to certain aspects, by resisting the pressure of one or more peristaltic waves at the intestinal site. The dosage form with the surface-loaded monolithic SPH body may thus be capable of providing sustained contact of the active agent on the SPH body surface with the intestinal tissue, thereby enhancing the bioavailability of active agents that may otherwise be poorly absorbable or otherwise difficult to administer via other forms.

Yet another advantage of embodiment of the dosage form and/or delivery method described herein may be to reduce the amount of active agent needed for agents which are required to be systemically available (that is, to enter the bloodstream) to be effective. For example, an agent that is only 40% bioavailable in a standard oral dosage form may have higher bioavailability when dosed as described according to embodiments disclosed herein. Higher oral bioavailability has the potential to reduce costs of the active agent, reduce side effects caused by active agent in the GI tract and to reduce the potential for development of side effects due to active agent remaining in the GI tract. Additionally, increasing the oral bioavailability of oral antibiotics has the potential to reduce the development of antibiotic drug resistance due to unabsorbed drug in the small intestine and colon.

According to yet further aspects, a SPH polymer composition has been developed that can exhibit characteristics such as swelling speed, swelling rate, radial pressure and/or compressive pressure that render it suitable for use in the dosage form. For example, the SPH polymer composition may provide for rapid and expansive swelling during deployment of the SPH monolith at the intestinal site, and with compressive and/or radial strength that may be adequate to retain the SPH body at the intestinal site, while also resisting rapid breakdown and/or transit of the SPH body away from the intestinal site that may be caused by peristaltic waves. Furthermore, without being limited to any one theory, it is believed that improved bioavailability of the active agent may be enhanced by the fluid uptake of the SPH at the intestinal site, which may increase the effective local concentration of the active agent, providing a greater driving force to transport the active agent across the intestinal wall. Additional potential benefits for bioavailability that may be imparted by SPH fluid uptake and/or presentation of the active agent near the mucosal surface, can include the fact that a smaller distance may be required for the active agent to diffuse from the dosage form to the mucosal surface, thus increasing its potential rate of absorption, and also providing for less duration of exposure of the active agent to the harsh and potentially degrading environment of the GI tract.

Detailed discussion of embodiments of the oral dosage form that are capable of enhancing active agent absorption and bioavailability is provided below.

Target Tissue

In one embodiment, the oral dosage form is configured to provide delivery of the active agent to a target tissue within the gastrointestinal tract, such as for example the upper gastrointestinal tract or the lower gastrointestinal tract (i.e., the small intestine or large intestine). For example, in one embodiment, the site of delivery of the active agent may be to the mucosa of the small intestine (e.g., the duodenum, jejunum, or ileum) and/or the large intestine (e.g., the ascending colon, the right colic flexure, the transverse colon, the transverse mesocolon, the left colic flexure, the descending colon, the sigmoid colon, and the rectum). In one embodiment, the oral dosage form is configured to provide delivery of the active agent to tissue in the ileum of the small intestine.

According to one embodiment, delivery to a particular region of the gastrointestinal tract, such as to a site in the small intestine, can be achieved by selecting the configuration and composition of the oral dosage form. For example, a protective coating such as an enteric coating can be provided that at least partially shields the dosage form during transit through the stomach and/or other areas of the upper gastrointestinal tract, until a predetermined location in the lower gastrointestinal tract is reached. Further discussion of embodiments of a protectively coated and/or enterically coated dosage form and/or other forms capable of delivering an active agent to a predetermined location in the gastrointestinal tract is provided in further detail below.

Dosage Form

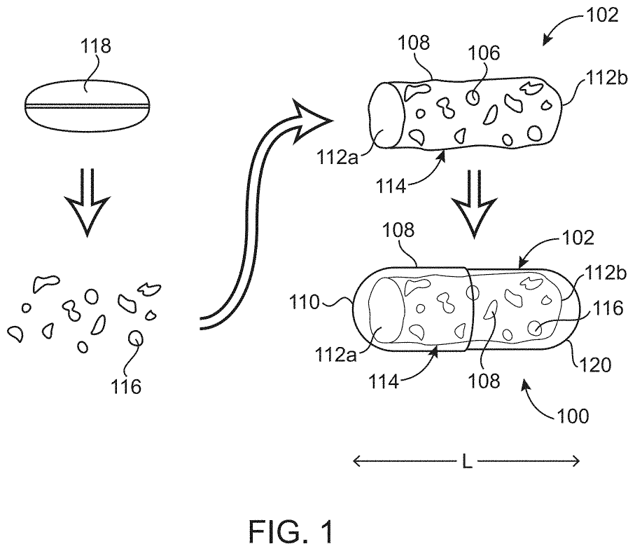

The pharmaceutically acceptably oral dosage form for delivery of an active agent to an intestinal site, according to embodiments of the present disclosure, may be capable of providing active agent into close contact with and/or in the vicinity of intestinal tissue at the target intestinal site, to promote uptake of the active agent at the target site. Referring to FIGS. 1-6, according to certain embodiments, the pharmaceutically acceptable oral dosage form 100 for delivery of the active agent to the intestinal site comprises a delivery structure 102 having a body 104 of superporous hydrogel (SPH) material, which body 104 according to certain aspects may be a monolithic body 104 of the SPH material. The body 104 further comprises an exterior surface 108 with one or more active agent delivery regions 106 thereon, for delivery of the active agent. The one or more active agent delivery regions 106 comprise a region of the exterior surface 108 where active agent is located, for delivery thereof with the oral dosage form. The oral dosage form 100 further comprises a protective coating 110 that covers at least a portion of the delivery structure 102, such as for example an enteric coating and/or timed-release coating that allows for deployment of the delivery structure 102 from the oral dosage form 100 at and/or in the vicinity of the target intestinal site.

According to one embodiment, the delivery structure 102 is configured such that the one or more active agent delivery regions 106 located at the exterior surface 108 of the body 104 are the primary source of active agent delivery from the dosage form 100. That is, according to certain embodiments, all or most of the active agent present in the dosage form may be located at the one or more active agent delivery regions 106 located at the exterior surface 108. Without being limited by any single theory, it is believed that by locating the active agent at the exterior surface 108 of the body 104, enhanced bioavailability of the active agent can be provided, for example as compared to dosage forms wherein the active agent is located internally within a delivery structure. Accordingly, in one embodiment, at least 10 wt % of the active agent contained in the oral dosage form 100 is located in the one or more active agent delivery regions 106 located at the exterior surface 108 of the body 104. According to further embodiments, even greater amounts of the active agent are located in the one or more active agent delivery regions 106 located at the exterior surface 108 of the body 104. For example, according to certain embodiments, at least 20 wt %, at least 30 wt %, at least 50 wt %, at least 60 wt %, at least 70 wt %, at least 80 wt %, at least 90 wt %, at least 95 wt %, at least 98 wt % and/or at least 99 wt % of the active agent contained in the oral dosage form 100 is located in one or more active agent delivery regions 108 at the exterior surface 106 of the body 104.

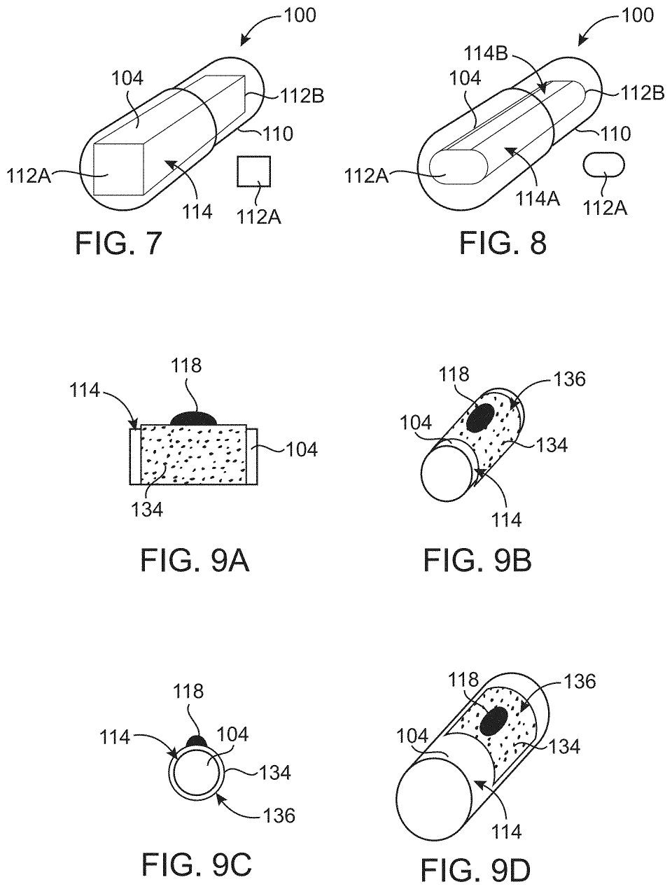

Referring to FIGS. 1-6, embodiments of oral dosage forms having the delivery structure 102 with the body 104 of SPH material, and active agent located at the exterior surface 108 thereof, is described in more detail. In the embodiments as shown, the body 104 comprises first and second ends 112a,b that are separated from one another along a longitudinal axis L of the body 104. The body 104 comprises a side surface 114 that extends between the first and second ends 112a,b, and that extends about the longitudinal axis L of the body 104. For example, in the embodiments as shown in FIGS. 1-6, the body 104 comprises an elongate side surface such as a cylindrically shaped side surface 114 extending between the first and second ends 112a,b, to form a cylindrically shaped body 104. In the embodiment shown in FIG. 7, the body 104 comprises a rectangular prism shape, with substantially flat panels on four sides making up the side surface 114 extending between the first and second ends 112a,b, to form the rectangular prism shaped body 104. In the embodiment shown in FIG. 8, the body 104 comprises an elongated shape with a side surface 114 comprising both curved 114a and substantially planar 114b portions. Other shapes and/or configurations of the body and/or surfaces thereof may also be provided, such as for example prismatic, rounded, spherical, hemispherical, cylindrical, half-cylindrical, oblong, and/other shapes, such as for example to provide a shape suitable for a tablet and/or capsule. For example, in one embodiment, the body may comprise a rounded and/or oblong shape, with rounded first and second ends 112a,b connected to a curved side surface 114. The first and second ends 112a,b may also be substantially flat, and/or the side surface 114 may comprise a series of flat panels that connect together to provide a prismatic structure. As yet a further embodiment, the body 104 can comprises a spherical shape, in which case the body 104 may comprise a rounded exterior surface without discernible first and second ends 112a,b, in which case the side surface 114 is effectively the entire surface of the body 104. According to aspects herein, the exterior surface 108 of any shape provided for the body 104 comprises the entire external surface of the body, meaning that the total area of the exterior surface 108 is the total surface area of the external surface of the body 104. For example, referring to FIGS. 1-6 depicting a cylindrically-shaped body, the exterior surface 108 includes the total surface of the side surface 114 and the surface of the first and second ends 112a,b, meaning that the total area of the exterior surface is the total of the surface area of the side surface 114 and the surface area of the first and second ends 112a,b.

According to certain embodiments, a body 104 having an elongated shape, such as a cylindrical, rectangular prism and/or oblong shape may be capable of providing advantageous effects in active agent delivery, such as for example by providing a shape that is capable of swelling to achieve a more conformal fit with the intestinal shape at the target site. Accordingly, according to certain embodiments, a ratio of a maximum length of the body 104, as measured according to a maximum distance between the first and second ends 112a,b in the longitudinal direction (i.e., parallel to the longitudinal axis L), to a maximum width of the body 104, as measured according to a maximum distance between opposing sides of the side surface 114 in a direction orthogonal to the longitudinal direction, is at least 1.25:1, such as at least 1.5:1, at least 1.75:1, at least 2:1, at least 2.5:1, and/or at least 3:1. Generally, the ratio of the maximum length to the maximum width will be less than 5:1, such as less than 4:1, and may even be less than 3:1. In the embodiments as shown in FIGS. 1-6, the elongated shape of the body 104 comprises a cylindrically shaped side surface 114 extending between first and second ends 112a, 112b. According to yet another embodiment, as shown in FIG. 7, the elongate shape of the body comprises a side surface 114 that is rectangular prism-shaped, such as by comprising four substantially planar surfaces that are at right angles to one another as shown, although other variations on elongated prismatic shapes may also be provided. A rectangular prism or other prismatic shape may be efficient to manufacture in certain embodiments, for example by preparing a bulk SPH material and slicing into individual prismatic shapes constituting the SPH body 104. In yet another embodiment as shown in FIG. 8, the elongate shape of the body comprises one or more substantially planar portions 114b of the side surface, in combination with one or more curved and/or cylindrical portions 114a of the side surface 114, such as for example in a machined cylindrical structure that has been machined to provide substantially planar surface regions 114b thereon (e.g., opposing first and second planar surface regions in the embodiment as shown in FIG. 8). The combination of curved and substantially planar surfaces may provide a structure that conforms nicely to the intestinal region upon swelling, while also providing substantially planar sites where it may be relatively easy to load active agent. For example, in one embodiment, the side surface 114 can comprise a substantially planar region 114b extending at least partly along the longitudinal axis of the monolithic body, and optionally extending between the first and second opposing ends 112a, 112b of the monolithic body.

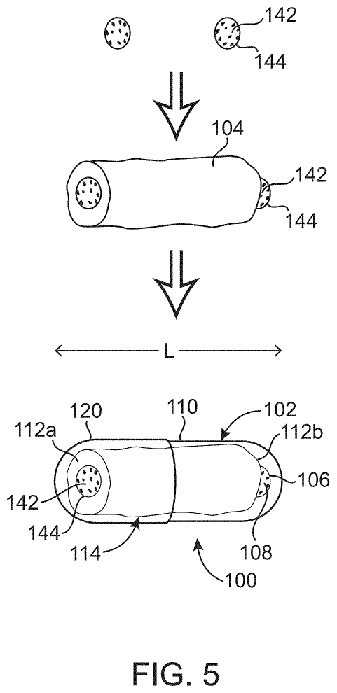

According to embodiments herein, the one or more active agent delivery regions 106 comprise regions of the exterior surface 108 where the active agent is located on the body 104. For example, according to some aspects, the one or more active agent regions 106 may be located on a side surface 114 of the body 104, such as for example to contact the active agent regions 106 having the active agent with neighboring intestinal tissue upon swelling of the body 104, as shown for example in FIG. 1. In one embodiment, the one or more active agent delivery regions 106 are located on a cylindrically shaped side surface 114 of the body 104, as shown for example in FIGS. 1-4 and 6. In another embodiment, the one or more active agent regions are located on a substantially planar side surface 114, as shown in FIG. 7, and/or on a substantially planar region 114b of the side surface 114, as shown for example in FIG. 8. According to yet another aspect, the one or more active agent regions 106 may be located at one or more first and second ends 112a,b of the body 104, as shown for example in FIG. 5. The one or more active agent regions 106 may also be provided across some combination of the side surface 114 and one or more longitudinal ends 112a,b, and/or further configurations of the active agent regions 106 on the exterior surface 108 may also be provided. The one or more active agent regions 106 may extend along a sufficient extent of the exterior surface to provide adequate delivery of the active agent to the target site from the regions 106. For example, according to certain embodiments, the one or more active agent delivery regions 106 can extend across at least 10%, at least 20%, at least 30%, at least 50%, at least 60%, at least 70%, at least 80%, at least 90%, at least 95%, at least 98% and/or at least 99% of the exterior surface 108 of the body 104, and may even substantially cover the entire exterior surface 108.

An embodiment of an oral dosage form 100 comprising the SPH body 104 is shown in FIG. 1. In the embodiment as shown in FIG. 1, the one or more active agent regions 108 comprise particles and/or granules 116 containing the active agent, which are disposed on the exterior surface 108, to provide delivery of the active agent at the target intestinal site. The particles and/or granules 116 may be adhered to the exterior surface 108, for example, by at least one of frictional forces and an adhering agent, such as a bio-compatible adhesive capable of binding the particles and/or granules 116 to the exterior surface 108. A suitable biocompatible adhesive could comprise, for example, any one or combination of a cyanoacrylate (“superglue”) ethylene vinylacetate (EVA), silicone and epoxy-based adhesives. According to one embodiment, the particles and/or granules may have an average particle and/or granule diameter size in a range of from 1 micron to 100 microns, such as in a range of from 10 microns to 80 microns. In some embodiments, the dosage form 100 can comprise particles and/or granules wherein at least 80%, 90%, 95%, and/or 99% of the particles and/or granules provided on the exterior surface 108 have a diameter size in a range from 1 micron to 100 microns, such as in a range of from 10 to 80 microns. According to certain embodiments, a maximum particle and/or granule diameter size provided to the SPH body 104 typically will not exceed 200 microns, and may not even exceed 150 microns, whereas a smallest particle and/or granule diameter size may be at least 0.05 microns, such as at least 0.5 microns. According to certain embodiments, the particles and/or granules 116 provided to the exterior surface 108 can comprise the main source of active agent in the dosage form. For example, the particles and/or granules 116 can comprise at least 10 wt %, at least 20 wt %, at least 30 wt %, at least 50 wt %, at least 60 wt %, at least 70 wt %, at least 80 wt %, at least 90 wt %, at least 95 wt %, at least 98 wt % and/or at least 99 wt % of the active agent contained in the dosage form.

Referring to FIG. 1, an embodiment of a method of preparing the dosage form 100 comprising the SPH body having the particles and/or granules at the exterior surface thereof is shown. In the embodiment as shown, a tablet 118 comprising the active agent is formed by compressing together a predetermined amount of active agent, optionally with excipients such as binder, other ingredients such as one or more permeation enhancers, and including further ingredients such as any described elsewhere herein. The compressed tablet 118 containing the active agent is then crushed or pulverized to form the smaller particles and/or granules 116. According to certain aspects, the particles and/or granules may thus themselves be compressed particles and/or granules containing the active agent, by virtue of having been formed as a part of the compressed tablet, and may thus allow for a significant amount of active agent and/or other ingredient, such as permeation enhancer, to be provided as a part of the particles and/or granules 116. While preparation of the particles and/or granules 116 is exemplified herein via crushing and/or pulverization of a compressed tablet, other means of preparation of the particles and/or granules may also be provided. For example, the particles and/or granules may be prepared from powdered materials such as active agent in powdered form, and optionally combined with one or more other ingredients such as permeation enhancer in powdered form, and other means of preparing the particles and/or granules may also be provided, such as fluidized bed pelletization.

Referring again to FIG. 1, the particles and/or granules 116 are provided to the exterior surface 108 of the SPH body 104, such as by rolling the SPH body 104 over the particles and/or granules to attach them thereto, or by otherwise coating the exterior surface 108 with the particles and/or granules 116. As discussed above, the particles and/or granules may be held to the exterior surface 108 by frictional forces, and/or an adhering agent may be applied to the exterior surface and/or particles to adhere them to the surface. In the embodiment as shown in FIG. 1, the particles and/or granules 116 are applied to a side surface 114 of the SPH body 104, such as a cylindrical and/or other elongate side surface. In other embodiments, the particles and/or granules may be applied to the exterior surface at one or more of the first and second ends 112a,b, and/or at a combination of the side surface and the first and second ends. In certain embodiments, the particles and/or granules may be applied substantially uniformly across the exterior surface 108, and/or may be applied according to a predetermined distribution across the exterior surface. Once the delivery structure 102 comprising the SPH body 104 with particles and/or granules 116 containing active agent attached to the exterior surface 108 thereof has been prepared, the delivery structure 102 can be provided with a protective coating 110 to protect the active agent and/or SPH body until delivery thereof can be made at the target site. In one embodiment, the delivery structure 102 may be contained inside a capsule 120 containing a protective coating 110 on an external surface thereof 122, such as an enteric coating, as is described in more detail below. In further embodiments, the capsule 120 may itself serve as the protective coating, and/or a protective coating 110 may be provided directly about the delivery structure 102 without any intervening capsule structure. Accordingly, in the embodiment as shown in FIG. 1, the dosage form 100 may provide a relatively easy to manufacture formulation with a relatively high amount of active agent at the exterior surface 108, and optionally with a relatively high amount of permeation enhancer in proximity to the active agent, that can be brought into contact with intestinal tissue at the target site upon deployment of the SPH body from the dosage form 100.

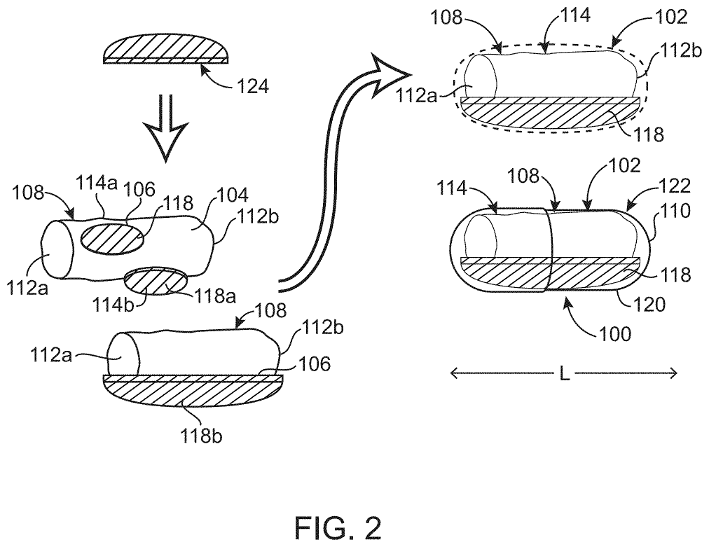

Referring to FIG. 2, a further embodiment of a dosage form 100 comprising the SPH body 104 and active agent at the exterior surface 108 thereof is described. In the embodiment as shown in FIG. 2, the one or more active agent delivery regions 106 comprise one or more compressed tablets 118 attached to the exterior surface 108 of the SPH body 104, the one or more compressed tablets 118 having the active agent contained therein as a part of the tablet composition. The one or more compressed tablets 118 may be affixed to the exterior surface 108 of the SPH body 104, for example, by providing a biocompatible adhesive, and/or by compressing the one or more tablets against and/or into the exterior surface 108 of the SPH body to adhere them thereto. The one or more tablets 118 may be sized and configured according to predetermined parameters for the dosage form 100, such as to provide for good delivery of the active agent, and/or to provide good adhesion to the SPH body 104. For example, in one embodiment, the dosage form 100 can comprise one or more mini-tablets 118a having a smaller tablet size that allows at least two or more to be affixed to the SPH body. According to one embodiment, the one or more compressed tablets 118 are affixed to the exterior surface 108 of the SPH body 104 such that they extend across at least 20%, at least 30%, at least 40%, at least 50%, at least 60%, at least 70%, at least 80%, at least 90%, at least 95%, at least 98%, and/or at least 99% of the exterior surface 108 of the body 104.

Referring to FIG. 2, an embodiment of a method of preparing the dosage form 100 comprising the SPH body having the one or more compressed tablets 118 adhered at the exterior surface 108 thereof is shown. In the embodiment as shown, one or more tablets 118 comprising the active agent are formed by compressing together a predetermined amount of active agent, optionally with excipients such as binder, other ingredients such as one or more permeation enhancers, and including further ingredients such as any described elsewhere herein. The one or more compressed tablets 118 containing the active agent may optionally be further fragmented to provide a predetermined size, such as by cleaving a full tablet to half-tablet size. In one embodiment, the one or more compressed tablets 118 comprise an engagement surface 124 that is configured to be affixed to the exterior surface 108 of the SPH body. The engagement surface 124 may be, for example a substantially planar surface and/or may be a surface having a slight curvature, such as in a case where a portion of the exterior surface 108 of the SPH body is curved, to provide a more conformal fit to the exterior surface 108. Depending on the size of the compressed tablet 118, the engagement surface 124 may engage with just a section of the exterior surface 108, as in the case of mini-tablets, and/or may engage substantially an entire side of the SPH body 104, such as an entire side of the SPH body along a longitudinal axis L thereof. According to certain aspects, the compressed tablet 118 may allow for a significant amount of active agent and/or other ingredient, such as permeation enhancer, to be provided as a part of the compressed tablet 118.

Referring again to FIG. 2, once the one or more tablets 118 have been prepared, the engagement surface(s) 124 of the tablets 118 are affixed to the exterior surface 108 of the SPH body 104, such as by adhering the engagement surface 124 to the exterior surface with a biocompatible adhesive, and/or by compressing the engagement surface 124 against and/or into the exterior surface 108. In the embodiment as shown in FIG. 2, the one or more tablets 118 are applied to a side surface 114 of the SPH body 104, such as a cylindrical and/or other elongate side surface. In other embodiments, the one or more tablets 118 may be applied to the exterior surface at one or more of the first and second ends 112a,b, and/or at a combination of the side surface and the first and second ends. In one embodiment as shown in FIG. 2, a tablet 118b that is a full-sized half-tablet is affixed along a cylindrical and/or other elongate side surface 114 of the SPH body, to substantially cover a portion of the side surface 114 extending between first and second ends 112a,b of the SPH body along the longitudinal axis. In this embodiment, deployment of the delivery structure 102 at the target site may result in swelling of the SPH body such that the tablet 118b along the side surface 114 of the SPH body is pressed into contact with the neighboring intestinal tissue, thereby improving uptake of the active agent in the tablet 118b via a uni-directional release of active agent from the tablet 118b. In yet another embodiment, one or more mini-tablets 118a are affixed at spaced-apart and even opposing surface portions 114a, b of the side surface 114, such as at opposing surface portions 114a,114b of a cylindrical and/or other elongate side surface 114. For example, the one or more mini-tablets may be spaced apart from one another about a circumference of the SPH body, such that the mini-tablets are affixed at different sides of the SPH body, and even at opposing sites about a circumference of the SPH body. In this embodiment, deployment of the delivery structure 102 at the target site may result in swelling of the SPH body such that the mini tablets 118a at the spaced apart positions along the side surface 114 of the SPH body are pressed into contact with the neighboring intestinal tissue on multiple sides of the SPH body, thereby providing a multi-directional release of the active agent from the mini-tablets 118a.

According to certain embodiments, once the delivery structure 102 comprising the SPH body 104 with one or more tablets 118 attached to the exterior surface 108 thereof has been prepared, the delivery structure 102 can be provided with a protective coating 110 to protect the active agent and/or SPH body until delivery thereof can be made at the target site. In one embodiment, the delivery structure 102 may be contained inside a capsule 120 containing a protective coating 110 on an external surface thereof 122, such as an enteric coating, as is described in more detail below. In further embodiments, the capsule 120 may itself serve as the protective coating, without requiring a separate coating. According to yet another embodiment, a protective coating 110 such as an enteric coating may be provided directly about the delivery structure 102 without any intervening capsule structure. Accordingly, in the embodiment as shown in FIG. 2, the dosage form 100 may provide a relatively easy to manufacture formulation with a relatively high amount of active agent at the exterior surface 108, and optionally with a relatively high amount of permeation enhancer in proximity to the active agent, that can be brought into contact with intestinal tissue at the target site upon deployment of the SPH body from the dosage form 100, and may be capable of providing unidirectional release and/or release in multiple directions of the active agent, according to the configuration thereof, to enhance delivery of the active agent therewith.

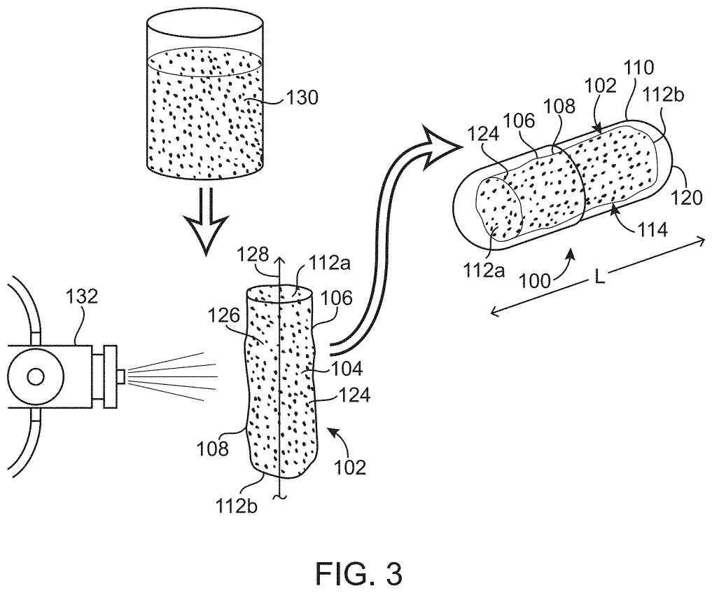

Referring to FIG. 3, a further embodiment of a dosage form 100 comprising the SPH body 104 and active agent at the exterior surface 108 thereof is described. In the embodiment as shown, the one or more active agent delivery regions 106 comprises a coating 124 containing the active agent that is formed across at least a portion of the exterior surface 108 of the SPH body 104. Aspects of the embodiment may thus provide for delivery of the active agent via direct intestinal apposition, to maximize contact of the active agent with tissue at the intestinal target site. According to one embodiment, the coating 124 containing the active agent extends across at least 25%, at least 30%, at least 50%, at least 60%, at least 70%, at least 80%, at least 90%, at least 95%, at least 98%, and/or at least 99% of the exterior surface of the SPH body 104. Furthermore, according to certain aspects, the coating 124 may at least partially permeate through the exterior surface 108 into a portion of the interior volume 126 of the SPH body 104. For example, in certain aspects, the coating 124 may at least partially permeate through the exterior surface 108 such that the coating extends a certain distance towards a center of the dosage form 100, such as towards a central axis 128 of the dosage form 100 that is aligned along the longitudinal axis L. In one embodiment, the coating 124 may permeate through the exterior surface 108 along a length towards the central axis 128 that is no more than 50% of the distance to the central axis, such as no more than 30%, no more than 20%, no more than 10%, no more than 5%, no more than 1%, and/or no more than 0.5% of the distance to the central axis 128 from the exterior surface 108. Furthermore, in certain embodiments the coating 124 may reside substantially at the exterior surface 108, with little or no penetration into an interior volume 126 of the SPH body 104. In one embodiment, the coating 124 contains at least 20 wt %, at least 30 wt %, at least 50 wt %, at least 60 wt %, at least 70 wt %, at least 80 wt %, at least 90 wt %, at least 95 wt %, at least 98 wt % and/or at least 99 wt % of the active agent contained in the dosage form 100.

According to certain aspects, the coating 124 may further comprise additional excipients and/or additives to enhance the dosage form 100, such as for example by improving delivery of the active agent. Furthermore, the coating 124 may according to certain aspects comprise a single layer or optionally multiple layers of the coating composition, and/or multiple layers each having different compositions may be provided to form the coating 124, according to predetermined delivery characteristics for the active agent. The coating 124 may be formed by a suitable coating method, such as by spray coating of the SPH body 104 with a coating composition, either in liquid or powdered form, immersing the SPH body 104 in a liquid or powdered coating composition, rolling the SPH body 104 in the coating composition, coating in a fluidized bed, and other suitable methods.

Referring again to FIG. 3, an embodiment of a method of forming a dosage form 100 comprising a coated SPH body 104 is described. In the embodiment as shown, a spray coating method is used to apply a liquid formulation of the coating composition to an exterior surface 108 of the SPH body 104. Other methods of forming the coating 124 may also be provided as an alternative and/or in addition to the spray coating method. According to the embodiment as shown, a coating solution 130 is prepared that contains the one or more active agents, and optionally one or more further additives such as for example permeation enhancer. The coating solution 130 can comprise a solution formulated to optimize application of the composition containing the active agent to the exterior surface 108, such as one or more liquids and/or additives to dissolve the active agent, and/or to provide a suspension of the active agent in solution. In one embodiment, the coating solution comprises an aqueous solution having the active agent and optionally permeation enhancer dissolved therein.

According to aspects herein, a coating device 132 is provided to apply the coating composition to exterior surface 108 of the SPH body 104. For example, the coating solution comprising an aqueous liquid formulation including the active agent and optionally permeation enhancer can be loaded in a coating device 132 comprising a spray coating device, to spray coat the liquid coating composition onto the exterior surface 108 of the SPH body 104. The resulting coating composition formed on the exterior surface 108 of the SPH body 104 may thus provide for good delivery of the active agent from the exterior surface 108 to intestinal tissue at the target site that is in the vicinity and/or even in contact with the exterior surface 108 by virtue of swelling of the SPH body 104.

According to certain embodiments, once the delivery structure 102 comprising the SPH body 104 with the coating 124 at the exterior surface 108 thereof has been prepared, the delivery structure 102 can be provided with a protective coating 110 to protect the active agent and/or SPH body until delivery thereof can be made at the target site. In one embodiment, the delivery structure 102 may be contained inside a capsule 120 containing a protective coating 110 on an external surface thereof 122, such as an enteric coating, as is described in more detail below. In further embodiments, the capsule 120 may itself serve as the protective coating, without requiring a separate coating. According to yet another embodiment, a protective coating 110 such as an enteric coating may be provided directly about the delivery structure 102 without any intervening capsule structure. Accordingly, in the embodiment as shown in FIG. 3, the dosage form 100 may provide a relatively easy to manufacture formulation with a relatively high amount of active agent at the exterior surface 108, to enhance delivery of the active agent to intestinal tissues at the target site.

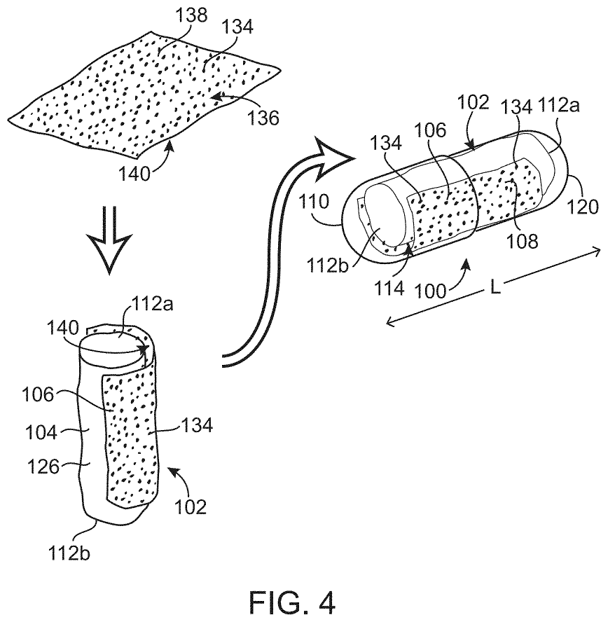

Referring to FIG. 4, a further embodiment of a dosage form 100 comprising the SPH body 104 and active agent at the exterior surface 108 thereof is described. In the embodiment as shown, the one or more active agent delivery regions 106 comprises one or more biodegradable films 134 comprising the active agent, the biodegradable film 134 being formed on at least a portion of the exterior surface 108 of the SPH body 104. The biodegradable film 134 may act to at least partially inhibit diffusion of the active agent into the interior volume 126 of the SPH body 104, such that active agent remains at the exterior of the delivery structure 102. According to certain embodiments, the biodegradable film 134 may contain the active agent disposed on an outer surface 136 thereof, such as for example by coating of the surface 136 of the biodegradable film 134 with any of the methods described herein, such as a spray coating method. According to yet another embodiment, the biodegradable film may contain active agent and optionally other additives such as permeation enhancer incorporated into the film formulation. In further aspects, the biodegradable film may be combined with other embodiments herein to inhibit diffusion of active agent into the interior volume 126 of the SPH body, such as in combination with the particles and/or granules described with reference to FIG. 1 and/or the compressed tablets described with reference to FIG. 2. That is, in certain embodiments, the active agent can be present in the form of one or more of granules and/or particles, compressed tablet, and/or lipid-containing composition, and is disposed on the outer surface of the biodegradable film. For example, FIGS. 9A-9D illustrate embodiments where the active agent is incorporated into a compressed tablet 118 that is disposed on an outer surface 136 of the biodegradable film 134, such as for example by adhering or otherwise placing adjacent to the outer surface 136. In one embodiment, the biodegradable film can comprise one or more film-forming biopolymers comprising at least one of proteins, polysaccharides (e.g., carbohydrate and gums), polypeptides, and lipids and/or other polymers that are compatible with biological use (see, e.g., Chapter 9—Edible Films and Coatings: A Review, in Innovations in Food Packaging (2^{nd} Ed), pages 213-255, Academic Press (2014)).

According to certain aspects, the biodegradable film 134 comprises a flexible and/or stretchable film that is capable of stretching and/or expansion to accompanying swelling of the SPH body 104. According to further aspects, the biodegradable film 134 may be a relatively non-stretchable film that is configured on the exterior surface 108 to allow swelling of the SPH body at the target site, such as for example by providing breaks in the film 134 that may accommodate swelling of the underlying SPH body 104, or by providing the biodegradable film across only a portion of the exterior surface 108, so as to not excessively constrict or restrain swelling of the underlying SPH body 104. For example, referring to FIG. 9B, the SPH body 104 is illustrated in an unswelled form where the biodegradable film encircles a substantial portion, and even the entire, longitudinal perimeter of the SPH body. By contrast, in FIG. 9D which shows a swelled SPH body 104, the biodegradable film covers only a portion of the perimeter of the SPH body 104, as the SPH body perimeter increases with swelling, such that the biodegradable film no longer extends across as much of the SPH body side surface. The biodegradable film may also be provided with perforations or other weakened areas that allow the film to accommodate the swelling SPH body by releasing or rupturing at the weakened regions with swelling of the SPH body. According to yet another embodiment, the biodegradable film may also be at least partially dissolvable when exposed to fluid at the target site, such as for example upon deployment of the delivery structure 102 comprising the swellable SPH body 104 at the target site, such that biodegradable film can dissolve away from the SPH body 104 following deployment without excessively inhibiting swelling of the SPH body 104. In certain embodiments, a single biodegradable film 134 or optionally a plurality of one or more biodegradable films 134 may be provided on at least a portion of the exterior surface 108, for example extending across at least 50%, at least 60%, at least 70%, at least 80%, at least 90%, at least 95%, at least 98%, and/or at least 99% of the exterior surface 108 of the SPH body 104. Furthermore, the biodegradable film 134 may be positioned on the exterior surface 108 to optimize delivery of the active agent to the target site, such as for example by positioning the biodegradable film 134 on portions of the exterior surface 108 that come into close proximity to and/or even contact intestinal tissue at the target site with deployment of the delivery structure 102 and swelling of the SPH body 104 at the target site. For example, in the embodiment as shown in FIG. 4, the SPH body 104 comprises first and second ends 112a,b opposing one another along the longitudinal axis L, and an elongate surface such as a cylindrical side surface 114 extending between the first and second ends 112a,b, with the biodegradable film covering at least a portion of the elongate side surface 114, such that swelling of the SPH body brings the biodegradable film 134 disposed on the cylindrical side surface into proximity and/or contact with tissue at the target site.

Referring again to FIG. 4, an embodiment of method of preparing the dosage form 100 having the biodegradable film 134 is described. In one aspect, the biodegradable film 134 is formed in the shape of a sheet 138 having a surface area sufficient to cover a predetermined portion of the exterior surface 108 of the SPH body 104. The sheet 138 can comprise one or more polymers making up the biodegradable film, along with active agent and optionally further additives such as permeation enhancer. In one embodiment, a biodegradable film 134 may be formed by combining active agent and optionally any further additive materials, with one or more biodegradable film polymeric materials, and curing to form the biodegradable film. According to another embodiment, the active agent and optionally further additives may be incorporated into the biodegradable film by soaking the biodegradable film in a solution containing the active agent and optional further additives. In yet another embodiment, the active agent and optional other additives can be provided to the outer surface 136 of the biodegradable film, such as by coating or otherwise affixing an active-agent containing composition to the biodegradable film, including by any of the coating and/or adhering methods described herein. Furthermore, while the embodiment depicted in FIG. 4 shows only a single biodegradable film 134, embodiments may further include providing a plurality of biodegradable films to the exterior surface 108 of the SPH body, and even a plurality of layers of biodegradable film. The biodegradable film 134 comprising the active agent can be placed on the exterior surface 108 of the SPH body 104, such as by wrapping the body 104 with the film 134, to place an inner surface 140 of the biodegradable film into contact with the exterior surface 108, and the biodegradable film may further optionally be adhered to the exterior surface 108. Alternatively and/or additionally, the biodegradable film 134 may be manufactured directly on the exterior surface of the SPH body, such as by dip coating or spray coating the biodegradable film 134 onto the exterior surface 108.

Similarly to the embodiments described above, once the delivery structure 102 comprising the SPH body 104 with the biodegradable film coating 134 at the exterior surface 108 thereof has been prepared, the delivery structure 102 can be provided with a protective coating 110 to protect the active agent and/or SPH body until delivery thereof can be made at the target site. In one embodiment, the delivery structure 102 may be contained inside a capsule 120 containing a protective coating 110 on an external surface thereof 122, such as an enteric coating, as is described in more detail below. In further embodiments, the capsule 120 may itself serve as the protective coating, without requiring a separate coating. According to yet another embodiment, a protective coating 110 such as an enteric coating may be provided directly about the delivery structure 102 without any intervening capsule structure. Accordingly, in the embodiment as shown in FIG. 4, the dosage form 100 may provide a relatively easy to manufacture formulation where a relatively high amount of active agent can be provided at the exterior surface 108, and diffusion of the active agent away from the surface is inhibited by the body of biodegradable film, to enhance delivery of the active agent to intestinal tissues at the target site.

Referring to FIG. 5, another embodiment of a dosage form comprising the active agent is provided. In the embodiment as depicted in FIG. 5, the active agent is incorporated into a lipid-containing composition 142 that is provided at least at a portion of the exterior surface 108 of the SPH body 104. The lipid composition may be, for example, a relatively hydrophobic (lipophilic) composition, that may provide a carrier for the active agent, while also inhibiting diffusion of the active agent into the relatively hydrophilic SPH body 104. For example, the lipid composition can comprise one or more of fatty acids, waxes, sterols, fat soluble vitamins, mono-, di- and/or triglycerides, and phospholipids. According to certain aspects, the lipids may be formulated to provide a suitable carrier for the active agent, such as according to the relative hydrophobicity and/or hydrophilicity of the active agent to be delivered. For example, the lipid formulation may be formulated as one or more of a liposome, micelle, an oil-in-water and/or water-in-oil formulation. One or more additional additives, such as for example a permeation enhancer, may also be incorporated into the lipid composition, to enhance delivery of the active agent.

In one embodiment, the lipid composition can further comprise lipophilic materials and/or vehicles such as one or more of an oil, gel, paste, semi-solid, wax, or other similar material, having the active agent dissolved or suspended therein. In one embodiment, the lipophilic vehicle may comprise a substance that is solid at room temperature, such as a wax, but that is at least partially in liquid form at physiological temperatures. According to one aspect, the lipophilic vehicle may be anhydrous, for example containing less than 1 wt % of water, and even less than 0.1 wt % of water, such as less than 0.01 wt % of water. In one embodiment, suitable materials for the lipophilic material can comprise one or more of castor oil, polyoxyalkylated sorbitol esters (such as TWEEN 80, a polyethylene sorbitol ester), mono-, di- and tri-glycerides of C₆ to C₂₂ saturated and unsaturated fatty acids, including glyceryl tricaprylate and glyceryl monocaprylate, mineral oil, a paraffin, a fatty acid, a mono-glyceride, a diglyceride, a triglyceride, an ether, and ester, olive oil, corn oil, coconut oil, peanut oil, soybean oil, cotton seed oil, sesame oil, canola oil, and combinations thereof.

According to certain embodiments, the lipid composition 142 containing the active agent can be provided on the exterior surface 108 of SPH body in a shape and/or configuration that provides for the improved delivery of the active agent contained within the lipid composition. For example, according to one embodiment, a layer of lipid composition containing the active agent may be coated on the exterior surface (not shown). According to another embodiment, the lipid composition may be provided at localized areas on the exterior surface 108 of the SPH body. For example, as shown in the embodiment illustrated in FIG. 5, the lipid composition 142 may be provided at one or more of the first and second ends 112a,b of an SPH body 104 having an elongate shape, such as a cylindrical shape, to provide for release of the lipid composition along with swelling of the SPH body 104. Furthermore, in the embodiment as illustrated in FIG. 5, the lipid composition is contained within one or more capsules 144 that are disposed on the exterior surface 108 of the SPH body 104. The capsules 144 may contain the lipid composition 142 to provide for deployment thereof at a predetermined position on the SPH body, and may further inhibit diffusion of the active agent into the SPH body. For example, as illustrated in the embodiment in FIG. 5, capsules may be disposed at each of the opposing first and second ends 112a,b of the SPH body 104 to contain the lipid composition having the active agent at the ends of the SPH body. Additionally and/or alternatively, one or more capsules 144 may be located at one or more positions on the side surface 114 of the SPH body, for example to provide contact of the lipid composition with tissue at the target site with swelling of the SPH body 104. The one or more capsules 110 may comprise a material that at least partially dissolves upon exposure to the environment in the gastrointestinal tract, similarly to an enteric coating, and/or may comprise another material configured to release the lipid composition 142 at the target site. As yet another embodiment, a protective coating 110 provided to encapsulate the delivery structure 102 comprising the SPH body and lipid composition 142 may itself serve to enclose and contain the lipid composition therewithin, without the addition of further capsules 144 within the protective coating 110.

Referring again to FIG. 5, a method of preparing the dosage form 100 comprising the lipid composition containing the active agent and SPH body is described. According to the embodiment as illustrated in FIG. 5, the lipid composition 142 is formulated with the active agent therein, and the composition is placed in capsules 144 that are sized to fit on first and second ends 112a,b of the SPH body 104. The capsules 144 are positioned on the exterior surface 108 of the SPH body 104, one on each opposing end. Optionally, further capsules 144 could be provided at other positions along the SPH body. The one or more capsules 144 can optionally be affixed to the SPH body, for example with a biocompatible adhesive. Once the delivery structure 102 comprising the SPH body 104 and capsules 144 is formed, the delivery structure 102 can be provided with a protective coating 110 to protect the active agent and/or SPH body until delivery thereof can be made at the target site. In one embodiment, the delivery structure 102 may be contained inside a capsule 120 containing a protective coating 110 on an external surface thereof 122, such as an enteric coating, as is described in more detail below. In further embodiments, the capsule 120 may itself serve as the protective coating, without requiring a separate coating. According to yet another embodiment, a protective coating 110 such as an enteric coating may be provided directly about the delivery structure 102 without any intervening capsule structure. Accordingly, in the embodiment as shown in FIG. 5, the dosage form 100 may provide a relatively easy to manufacture formulation that inhibits diffusion of active agent into the SPH body and thus promotes release of the active agent at the target site of the intestinal tissue, to enhance delivery of the active agent.

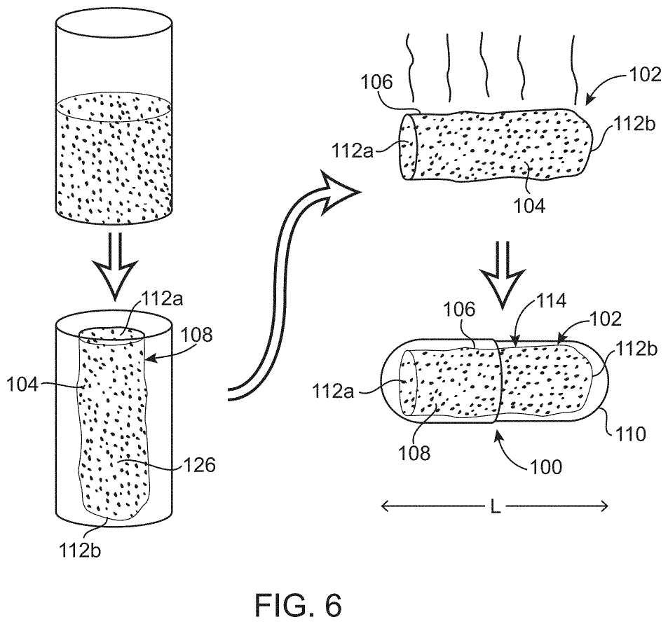

According to an embodiment as shown in FIG. 6, the active agent may also be provided to the SPH body 104, either in addition to any of the other methods described herein or alone, by soaking the SPH body 104 in a solution of the active agent to allow the active agent to diffuse into an interior volume 126 of the SPH body 104. While the embodiment may provide a relatively easy to manufacture dosage form 100, aspects of the embodiment may also be combined with any of the other embodiments described herein, in the interests of providing a relatively increased amount of active agent at the exterior surface 108 as opposed to within the interior volume 126, to enhance delivery of the active agent from the exterior surface 108. As yet another example, a solution containing the active agent may be permitted to permeate only a certain distance towards the interior volume 126 of the SPH body 104. For example, only the exterior surface of the SPH body may be exposed to the liquid solution containing the active agent, and/or the SPH body may be exposed to only a very small amount of liquid solution containing the active agent that is inadequate to fully permeate the SPH body. In one embodiment, the SPH body 104 may be exposed to liquid containing active agent under conditions such that the active agent permeates through the exterior surface 108 along a length towards a central axis 128 of the SPH body 104 that is no more than 50% of the distance to the central axis, such as no more than 30%, no more than 20%, no more than 10%, no more than 5%, no more than 1%, and/or no more than 0.5% of the distance to the central axis 128 from the exterior surface 108. One method of preparing the dosage form 100 having active agent loaded by exposing the exterior surface 108 of an SPH body to a liquid solution comprising active agent, is illustrated in FIG. 6. In the method as illustrated, a liquid composition comprising the active agent, and optionally further additives such as permeation enhancer, is prepared, such as for example by dissolving or suspending the active agent in the liquid solution. At least a portion of the exterior surface 108 is exposed to the active agent-containing solution, such as for example by immersing in the liquid solution. The delivery structure 102 comprising the SPH body with active agent loaded therein may then be allowed to dry to release excess liquid, and may even be subjected to a drying process involving heating and/or removal of excess liquid under negative pressure. The delivery structure can then be encapsulated in a protective coating 110, such as for example as described in the other embodiments herein.

According to certain embodiments, the body 104 of SPH provided as a part of the dosage form 100 is formed to have a substantially uniform exterior surface that provides good delivery of the active agent, such as an exterior surface that is substantially absent large surface indentations and/or voids where active agent might otherwise accumulate and/or that could impede access of the active agent from the body surface to the target delivery site. For example, in one embodiment, in a case that the body 104 comprises a surface indentation or void formed therein that is in connection with the exterior surface 108, such indentation and/or void has a volume that does not exceed a certain total volume occupied by the body, such as an indentation and/or void that does not exceed 30%, 20%, 10%, 8%, 7.5%, 7%, 6%, 5%, 3.5%, 3%, 1.5%, 1% and/or 0.5% of the total volume of the body. According to one embodiment, in a case where the body 104 comprises one or more surface indentations and/or voids formed therein in connection with the exterior surface, such as two or more surface indentations and/or voids formed therein, the one or more indentations and/or voids have a total combined volume that does not exceed 30%, 20%, 10%, 8%, 7.5%, 7%, 6%, 5%, 3.5%, 3%, 1.5%, 1% and/or 0.5% of the total volume occupied by the body. According to yet another embodiment, in a case where the body comprises one or more indentations or voids formed therein that are in connection with the exterior surface, the volume of such void or hole does not exceed 40 mm³, 30 mm³ and/or 20 mm³. In yet another embodiment, a total volume of any surface indentations and/or voids connected to the exterior surface and having a volume greater than 40 mm³, 50 mm³ and/or 65 mm³ does not exceed 30%, 20%, 10%, 8%, 7.5%, 7%, 6%, 5%, 3.5%, 3%, 1.5%, 1% and/or 0.5% of the total volume occupied by the body. Furthermore, according to certain embodiments, the one or more active agent regions 106 may be configured to limit the amount of active agent that is present in any such indentations and/or internal voids. For example, an amount of active agent present in any surface indentation and/or void connected to the exterior surface and having a volume greater than 40 mm³, 50 mm³ and/or 65 mm³, may be less than 50 wt %, less than 40 wt %, less than 30 wt %, less than 20 wt %, less than 10 wt %, less than 8 wt %, less than 5 wt %, less than 3 wt %, less than 2 wt %, less than 1.5 wt %, less than 1 wt %, less than 0.5 wt %, and/or less than 0.1 wt %.

Furthermore, in certain embodiments, the SPH body 104 comprises a single monolithic body of SPH. That is, the SPH body provided to impart swelling in the dosage form may consist of a single unitary and monolithic body, as opposed to multiple different SPH pieces. A single SPH body may provide a more uniform swelling and be more resistant to intestinal pressures. In alternative embodiments, the dosage form 100 can comprise a plurality of SPH bodies 104, such as two or three SPH bodies, each of which can comprise the active agent provided to the exterior surface thereof 108 via any of the embodiments described above (e.g., in FIGS. 1-6), or which bodies may be combined and/or adhered together such a shared exterior surface 108 extending across the plurality of SPH body comprises the exterior surface to which the active agent is provided. In one embodiment, the SPH body comprises a minimum size that imparts good swelling and/or other characteristics to enhance active agent administration, either as a single SPH body and/or optionally in combination with one or more other SPH bodies in the dosage form 100. For example, in one embodiment, the SPH body comprises a minimum diameter as measured orthogonal to a central axis of at least 4.5, at least 5 mm, as at least 6 mm, at least 8 mm, at least 9 mm, and/or at least 10 mm. In another embodiment, the SPH body can comprise a length, as measured between opposing longitudinal sides 112a,b of the body, of at least 8 mm, at least 10 mm, at least 12 mm, at least 15 mm, at least 20 mm, and/or at least 30 mm.

As discussed above, in certain embodiments the dosage form 100 can comprise a single body of SPH having size and swelling characteristics to impart advantageous active agent delivery properties. For example, in one embodiment, the dosage form 100 can comprise a single body of SPH that makes up a significant portion of all SPH provided in the dosage form, such as a single body of SPH comprising at least 20% by weight, at least 30% by weight, at least 50% by weight, at least 60% by weight, at least 70% by weight, at least 80% by weight, at least 90% by weight, at least 95% by weight, at least 98% by weight, and/or at least 99% by weight of the total amount of super porous hydrogel in the dosage form 100. The one or more SPH bodies 104 provided in the oral dosage form 100 can further comprise any of the shapes described herein, either as a single SPH body, or as combined with further SPH bodies in the dosage form 100. For example in one embodiment, the SPH body comprises the first and second opposing longitudinal end surfaces 112a, b, and a side surface 114 extending about a longitudinal axis of the body 104 that passes through the opposing first and second longitudinal end surfaces 112a,b. For example, the side surface can comprise an elongate side surface such as a cylindrical side surface, a rectangular or other prismatic side surface, an arcuate side surface 114 or other shape such as any of those described herein. As yet another option, the one or more SPH bodies 104 provided in the dosage form 100 may comprise a spherical shape, and/or may be layered with respect to one another, and/or form segments of SPH body that are connected together to give a larger SPH structure. For example, a plurality of cylindrically or other elongated shaped SPH bodies can be aligned to provide a large SPH structure with an overall cylindrical and/or elongate shape. Furthermore, according to one embodiment, one or more of the SPH bodies may comprise crevices therein to accommodate intestinal pressures on the SPH body and allow for disintegration of the SPH body after a predetermined period of time has elapsed with the SPH body deployed at the target site.

Active Agent

The oral dosage form according to embodiments of the present disclosure is adapted to deliver any of a wide range of active agents to a tissue site. Thus, for example, the oral dosage form may be adapted to deliver a single active agent or multiple active agents (e.g., two, three or more active agents, either serially or simultaneously) to the tissue site. Additionally, the active agents may be in any of a wide range of alternative forms such as pharmaceutically acceptable salt forms, free acid forms, free base forms, and hydrates.

In general, the active agent may be in particulate, liquid, or gel form and may comprise any of a range of compositions having biological relevance, e.g., metals, metal oxides, peptides, peptides structurally engineered to resist enzymatic degradation, antibodies, hormones, enzymes, growth factors, small organic molecules, ligands, or other pharmaceuticals, nutraceuticals, or biologics. In some embodiments, the agent(s) may include one or more large molecules (e.g., proteins and/or protein conjugates), and/or one or more small molecules (e.g., small organic molecules, and/or small peptides) as the agent(s). In one exemplary embodiment, the active agent comprises at least one polypeptide and/or small molecule having a therapeutic treatment effect. Examples of active agents that can be delivered by the oral dosage form can include at least one of octreotide, calcitonin (including salmon calcitonin), parathyroid hormone (PTH), teriparatide (a recombinant form of PTH) insulin, peptide agonists of GLP-1, such as exenatide, liraglutide, lixisenatide, albiglutide and/or dulaglutide, GLP-1/GIP co-agonists, GLP-2 agonists and peptide GPCR agonists. Additional examples of active agents include antibiotics such as azithromycin, vancomycin, dalbavancin (Dalvance), micafungin (Mycamine), Brilicidin, Avidocin, Purocin, and Arenicin. Active agents may also include the antimycobacterial agents clofazimine, ethionamide, para-aminosalicylic acid, and Amikacin.

In yet another embodiment, the active agent can comprise other large molecules and/or other structures other than those specifically listed above, such as for example any one or more of antibodies (monoclonal and polyclonal) or antibody fragments, polysaccharides, carbohydrates, nanoparticles, vaccines, biologics, nucleic acids, cells and cell therapies, DNA, RNA, siRNA, blood factors, gene therapies, thrombolytic agents (tissue plasminogen activator), growth factors (erythropoietin), interferons, interleukin-based molecules, fusion proteins, recombinant proteins, therapeutic enzymes, and others. The active agent may also and/or alternatively comprise at least one of a small molecule drug, a drug conjugate, a prodrug, a small organic molecule (e.g., with a molecular weight of about 500 Da or less), a metabolically activated agent (e.g., a metabolite), a nutrient, a supplement, and the like.

According to one embodiment, the oral dosage form is capable of providing improved bioavailability in delivering an active agent that may be otherwise incompletely absorbed in the intestine. For example, the oral dosage form having the SPH composition can be capable of providing surprisingly improved bioavailability for polypeptides and/or other small molecules having a relatively high molecular weight, which agents may be otherwise difficult to effectively administer due to their relatively large size. Examples of such active agents may include polypeptides and/or small molecules having a size of at least about 450 Da. However, according to one embodiment, the molecular weight of the active agent may still be below about 200,000 Da, to allow for good delivery/absorption of the active agent in the intestine. According to one example, in one embodiment the active agent has a molecular weight of at least about 2000 Da. By way of further example, in one embodiment the active agent has a molecular weight of at least about 5000 Da. By way of yet a further example, in one embodiment the active agent has a molecular weight of at least about 10,000 Da. While the active agent according to one embodiment will generally have a molecular weight below about 600,000 Da, as has been described above, the molecular weight may also in one example be below about 200,000 Da, such as below about 100,000 Da. For example, the active agent provided as a part of the oral dosage form may have a molecular weight in one embodiment that is in the range of from about 450 Da to about 500,000 Da, such as about 450 Da to about 25,000 Da, and even 450 Da to 10,000 Da, such as about 450 Da to about 6000 Da. For example, in one embodiment the active agent may have a molecular weight in a range of from about 1000 Da to about 25,000 Da, and even about 1,000 Da to about 10,000 Da, such as about 1000 Da to 5000 Da. As previously noted, the oral dosage form may contain two or more agents independently selected from molecules having a molecular weight within the ranges recited herein.

The oral dosage form comprises the at least one active agent in an amount or concentration that is suitable for the delivery of the active agent. For example, in one embodiment, a total content of the active agent in the dosage form may be at least about 0.0001% of the weight of the oral dosage form. By way of further example, in one embodiment, a total content of the active agent may be at least about 0.001% of the weight of the oral dosage form. By way of further example, in one embodiment, a total content of the active agent may be at least about 0.01% of the weight of the oral dosage form. By way of further example, in one embodiment, the active agent may be at least about 0.1% of the weight of the oral dosage form. By way of further example, in one embodiment, the active agent may be at least about 1% of the weight of the oral dosage form. By way of further example, in one embodiment, the active agent may be at least about 10% of the weight of the oral dosage form. By way of further example, in one embodiment, the active agent may be at least about 20% of the weight of the oral dosage form. By way of further example, in one embodiment, the active agent may be at least about 50% of the weight of the oral dosage form. By way of further example, in one embodiment the active agent is less than about 90% by weight of the oral dosage form. By way of further example, in one embodiment the active agent is less than about 25% by weight of the oral dosage form. By way of further example, in one embodiment the active agent is less than about 10% by weight of the oral dosage form. By way of further example, in one embodiment the active agent is less than about 5% by weight of the oral dosage form. In certain embodiments, the active agent may be between about 0.0001% and about 90% of the weight of the oral dosage form. By way of further example, in one embodiment, the active agent may be between about 0.01% and about 25% of the weight of the oral dosage form. By way of further example, in one embodiment, the active agent may be between about 1% and about 25% of the weight of the oral dosage form.

The content of the active agent in the oral dosage form can be selected according to the intended dose of the active agent to be provided, as well as the activity of the active agent. For example, in one embodiment, an active agent corresponding to octreotide may be provided in a content of at least about 0.3% of the weight of the oral dosage form. By way of further example, in one embodiment, the octreotide may be at least about 2.5% of the weight of the oral dosage form. By way of further example, in one embodiment, the octreotide may be at least about 5% of the weight of the oral dosage form. By way of further example, in one embodiment, the octreotide may be at least about 10% of the weight of the oral dosage form. In one embodiment the octreotide is provided in an amount of less than about 50% of the weight of the oral dosage form. By way of further example, in one embodiment the octreotide is less than about 25% of the weight of the oral dosage form. By way of further example, in one embodiment the octreotide is less than about 10% by weight of the oral dosage form. By way of further example, in one embodiment the octreotide is less than about 5% by weight of the oral dosage form. In certain embodiments, the octreotide may be between about 0.5% and about 50% of the weight of the oral dosage form. By way of further example, in one embodiment, the octreotide may be between about 2.5% and about 25% of the weight of the oral dosage form. By way of further example, in one embodiment, the octreotide may be between about 2.5% and about 10% of the weight of the oral dosage form.

In yet another embodiment, an active agent corresponding to calcitonin may be provided in a content of at least about 0.3% by weight of the oral dosage form. By way of further example, in one embodiment, the calcitonin may be at least about 2.5% of the weight of the oral dosage form. By way of further example, in one embodiment, the calcitonin may be at least about 5% of the weight of the oral dosage form. By way of further example, in one embodiment, the calcitonin may be at least about 10% of the weight of the oral dosage form. By way of further example, in one embodiment the calcitonin is less than about 50% by weight of the oral dosage form. By way of further example, in one embodiment the calcitonin is less than about 25% by weight of the oral dosage form. By way of further example, in one embodiment the calcitonin is less than about 10% by weight of the oral dosage form. By way of further example, in one embodiment the calcitonin is less than about 5% by weight of the oral dosage form. In certain embodiments, the calcitonin may be between about 0.5% and about 50% of the weight of the oral dosage form. By way of further example, in one embodiment, the calcitonin may be between about 2.5% and about 25% of the weight of the oral dosage form. By way of further example, in one embodiment, the calcitonin may be between about 2.5% and about 10% of the weight of the oral dosage form.

In another embodiment, an active agent corresponding to teriparatide may be provided in a content of at least about 0.3% by weight of the oral dosage form. By way of further example, in one embodiment, the teriparatide may be at least about 2.5% of the weight of the oral dosage form. By way of further example, in one embodiment, the teriparatide may be at least about 5% of the weight of the oral dosage form. By way of further example, in one embodiment, the teriparatide may be at least about 10% of the weight of the oral dosage form. By way of further example, in one embodiment the teriparatide is less than about 50% by weight of the oral dosage form. By way of further example, in one embodiment the teriparatide is less than about 25% by weight of the oral dosage form. By way of further example, in one embodiment the teriparatide is less than about 10% by weight of the oral dosage form. By way of further example, in one embodiment the teriparatide is less than about 5% by weight of the oral dosage form. In certain embodiments, the teriparatide may be between about 0.5% and about 50% of the weight of the oral dosage form. By way of further example, in one embodiment, the teriparatide may be between about 2.5% and about 25% of the weight of the oral dosage form. By way of further example, in one embodiment, the teriparatide may be between about 2.5% and about 10% of the weight of the oral dosage form.

In another embodiment, an active agent corresponding to exenatide may be provided in a content of at least about 0.001% by weight of the oral dosage form. By way of further example, in one embodiment, the exenatide may be at least about 0.01% of the weight of the oral dosage form. By way of further example, in one embodiment, the exenatide may be at least about 0.1% of the weight of the oral dosage form. By way of further example, in one embodiment, the exenatide may be at least about 1% of the weight of the oral dosage form. By way of further example, in one embodiment the exenatide is less than about 10% by weight of the oral dosage form. By way of further example, in one embodiment the exenatide is less than about 1% by weight of the oral dosage form. By way of further example, in one embodiment the exenatide is less than about 0.1% by weight of the oral dosage form. By way of further example, in one embodiment the exenatide is less than about 0.01% by weight of the oral dosage form. In certain embodiments, the exenatide may be between about 0.001% and about 10% of the weight of the oral dosage form. By way of further example, in one embodiment, the exenatide may be between about 0.01% and about 1% of the weight of the oral dosage form. By way of further example, in one embodiment, the exenatide may be between about 0.01% and about 0.1% of the weight of the oral dosage form.

In yet another embodiment, an active agent corresponding to liraglutide may be provided in a content of at least about 0.3% by weight of the oral dosage form. By way of further example, in one embodiment, the liraglutide may be at least about 2.5% of the weight of the oral dosage form. By way of further example, in one embodiment, the liraglutide may be at least about 5% of the weight of the oral dosage form. By way of further example, in one embodiment, the liraglutide may be at least about 10% of the weight of the oral dosage form. By way of further example, in one embodiment the liraglutide is less than about 50% by weight of the oral dosage form. By way of further example, in one embodiment the liraglutide is less than about 25% by weight of the oral dosage form. By way of further example, in one embodiment the liraglutide is less than about 10% by weight of the oral dosage form. By way of further example, in one embodiment the liraglutide is less than about 5% by weight of the oral dosage form. In certain embodiments, the liraglutide may be between about 0.5% and about 50% of the weight of the oral dosage form. By way of further example, in one embodiment, the liraglutide may be between about 2.5% and about 25% of the weight of the oral dosage form. By way of further example, in one embodiment, the liraglutide may be between about 2.5% and about 10% of the weight of the oral dosage form.

Super-Porous Hydrogel

As discussed above, in one embodiment the oral dosage form comprises a body having superporous hydrogel (SPH) composition that is capable of absorbing fluid at the target intestinal site, such that the SPH body swells at the intestinal site to bring active agent at the exterior surface of the SPH body into the vicinity of and even in contact with intestinal tissue at the target site. The swelling characteristics of the SPH body, and embodiments of polymer compositions for the SPH body, are described in more detail below.

According to one embodiment, the SPH composition used to form the SPH body can comprise a 3-dimensional network of hydrophilic polymers that forms a highly porous structure. In certain embodiments, a superporous hydrogel (SPH) material may have pore sizes of at least 0.5 microns to at least 10 microns, such as up to 80 microns, or even 200 microns or larger, although the pore size is typically less than about 1 mm. However, SPH materials may also come in a variety of different pore sizes, pore distributions, pore shapes, etc., and so the SPH materials as described herein are not limited to any one particular pore size and/or distribution. In certain embodiments, the SPH composition may generally be formed by combining polymerizable monomers with cross-linking agents, and initiators in aqueous solution, with materials that are conducive to forming a foamed composition while polymerization is taking place, such as foam stabilizers, foaming aids, and foaming agents, although other methods may also be provided. SPH compositions can comprise polymeric structures formed from polymerization of monomers with a cross-linking agent, and can also comprise polymeric structures formed from polymerization of monomers with a cross-linking agent in the presence of a swellable filler, which is also referred to as an SPH composite (SPHC), as well as SPH hybrids (SPHH) that use a hybrid agent, as discussed in “Recent Developments in Superporous Hydrogels” by Omidian et al. (J. of Pharmacy and Pharmacology, 59: 317-327 (2007)), which is hereby incorporated by reference herein in its entirety.

In particular, the superporous hydrogels may have a three-dimensional cross-linked network containing large numbers of interconnected and open pores, that may be capable of absorbing fluid rapidly to swell a in size a significant amount in a short period of time. Examples of materials that may be used to form polymeric networks for superporous hydrogels can include any one or more of acrylic acid, acrylamide, sodium acrylate, 2-hydroxyethyl methacrylate, 2-acrylamido-2-methyl-1-propanesulfonic acid, 2-acryloyloxy ethyl trimethylammonium methyl sulfate, 2-hydroxypropyl methacrylate, 3-sulphopropyl acrylate potassium, hydroxyl ethyl methyl acrylate, N-isopropyl acrylamide, acrylonitrile, polyvinyl alcohol, glutaraldehyde, N, N-methylenebisacrylamide, N, N, N, N-tetramethylenediamine, pluronic F127, hydroxyethyl acrylate, diethylene glycol diacrylate, polyethylene glycol acrylate, polyethylene glycol diacrylate, cross-linked sodium carboxymethylcellulose (Ac-Di-Sol), crosslinked sodium starch glycolate (Primojel), crosslinked polyvinylpyrrolidone (crospovidone), Carbopol, sodium alginate, sodium carboxymethylcellulose, chitosan, pectin. For example, superporous hydrogels can be formed using various hydrophilic polymers, such as one or more of poly(acrylic acid-co-acrylamide) (poly(AA-co-AM), poly(AA-co-AM) coated with poly(ethyleneglycol-b-tetramethylene oxide, or grafted with poly(ethylene glocol), or semi or fully-interpenetrated with chitosan or polyethyleneimine, or sodium alginate, poly(acrylamide), poly(acrylic acid), glycol chitosan, polysaccharides, starches, and the like. In one embodiment, the super porous hydrogel comprises a polymer formed from cross-linking a hydrophilic polymer using a polycarboxylic acid as a cross-linking agent. For example, the hydrophilic polymer can comprise a polysaccharide such as a cellulose or cellulose derivative, such as an alkylcellulose (e.g. methylcellulose, ethycellulose and n-propylcellulose), substituted alkyl-celluloses (e.g., hydroxyethylcellulose, hydroxypropylmethylcellulose and carboxymethylcellulose), a hydroxycellulose, a starch or starch derivative, dextran, glycosaminoglycans, polyuronic acids, and the like. The polycarboxylic acid can comprise an organic acid having two or more carboxylic acid functional groups, such as dicarboxylic acids such as oxalic acid, malonic acid, maleic acid, malic acid, succinic acids, and the like, and tricarboxylic acids such as citric acid, isocitric acid, aconitic acid, phthalic acid, and the like. In one embodiment, the superporous hydrogel can comprise a hydrophilic polymer corresponding to carboxymethylcellulose cross-linked with citric acid, and/or a combination of hydrophilic polymers including carboxymethylcellulose and hydroxyethylcellulose cross-linked by citric acid, as described for example in U.S. Pat. Nos. 8,658,147, 9,353,191, and U.S. PG-Pub No. 2014/0296507, all of which are incorporated by reference herein in their entireties.

The relative amount of void space in the SPH body can be at least indirectly assessed via the Effective Density of the SPH body in the Dried State, which is a measure of the mass of the SPH body per volume of the SPH body as measured using its external dimensions. The SPH body will typically have an Effective Density in the Dried State that is less than 1 g/cm³, such as less than 0.9 g/cm³, less than 0.8 g/cm³, less than 0.75 g/cm³, less than 0.6 g/cm³, less than 0.5 g/cm³, less than 0.45 g/cm³, less than 0.3 g/cm³, and/or less than 0.25 g/cm³. The Effective Density of the SPH body may typically be at least 0.05 g/cm³. Furthermore, in certain embodiments the Effective Density of the SPH body may be that for the SPH body in an Uncompressed State, whereas the SPH body in a Compressed State may have a significantly increased density over the same SPH body in the Uncompressed State.

For example, the Effective Density of the SPH body in a compressed state, such as to a state where the SPH body has a Compressed Volume that is less than 85%, less than 75%, less than 60% and/or less than 50% of an Uncompressed Volume in the Uncompressed State, may be closer to 1 g/cm³, such as at least 0.8 g/cm³ and/or at least 0.9 g/cm³, and may be at least twice and/or at least 3 times and/or even at least 4 times as high as the Effective Density of the SPH body in the Uncompressed State.

According to one embodiment, the SPH composition, such a monolithic SPH body comprising the composition, comprises a significant content of the dosage form as a percent by weight. For example, in one embodiment, the SPH composition and/or monolithic SPH body comprising the composition can comprise at least 20% by weight of the dosage form, such as at least about 30% by weight of the oral dosage form, at least 40 by weight of the dosage form, at least 50% by weight of the dosage form, at least 60% by weight of the dosage form, at least 60%, and/or at least 75% by weight of the dosage form. Similarly, the SPH composition, such as a monolithic SPH body comprising the SPH composition, may make up a significant portion of the volume of the dosage form, such as at least 20 volume %, at least 35 volume %, at least 50 volume %, at least 65 volume %, at least 75 volume %, at least 80 volume %, at least 90 volume %, and/or at least 95 volume % of the dosage form. In another embodiment, the SPH body comprises a mass of at least 50 mg, at least 75 mg and/or at least 100 mg, and no more than 2 g, no more than 1 g and/or no more than 0.5 grams.

According to one embodiment, it has been found that by providing an SPH body having certain properties in an oral dosage form, improved delivery of an active agent can be provided. For example, in one embodiment, the SPH body comprises a Maximum Swelling Ratio (i.e., a Swelling Ratio as measured at a time interval of 10 minutes after introducing fluid to the SPH material) that provides for swelling of the SPH body at the target intestinal site, to a size that places the active agent in close proximity to the intestinal tissue to enhance delivery thereto. In one embodiment, the SPH body comprises a Maximum Swelling Ratio of at least 20, at least 25, at least 35, at least 40, at least 45, at least 50, at least 55, at least 60, at least 65, at least 70, at least 75, at least 80, at least 85, at least 90, at least 95, at least 100, at least 115, at least 120, at least 130, at least 140, at least 150, at least 160, at least 170, at least 180, at least 190, at least 200, and/or at least 250. For example, the Maximum Swelling Ratio may be in a range of from 30 to 100, such as from 40 to 80, and even from 50 to 75.

As yet another example, the Swelling Speed of the SPH body, for example as measured by a Swell Ratio Percentage at a select time interval (e.g., at 1 minute after introduction of fluid to the SPH material), can be provided that allows for rapid deployment and swelling of the SPH body at the target site, thereby reducing the likelihood that the SPH body will be swept away by peristaltic or other forces before a Maximum Swell Ratio can be achieved. For example, in one embodiment, the SPH body comprises a Swell Ratio Percentage of at least 30%, at least 35%, at least 45%, at least 50%, at least 55%, at least 60%, and/or at least 70% of a Maximum Swell Ratio for the SPH material at a time interval of 60 seconds or less. According to yet another embodiment, the SPH body comprises a Swelling Ratio Percentage of at least 30%, at least 35%, at least 45%, at least 50%, at least 55%, at least 60% and/or at least 70% of a Maximum Swell Ratio for the SPH material at a time interval of 30 seconds or less. In yet another embodiment, the SPH body comprises a Swelling Speed in which a SPH Swelling Ratio Percentage of at least 30%, at least 35%, at least 45%, at least 50%, at least 55%, at least 60% and/or at least 70% of a Maximum Swell Ratio for the SPH material at a time interval of 90 seconds or less. In yet another embodiment, the SPH body comprises a swelling speed in which the SPH Swelling Ratio Percentage of at least 30%, at least 35%, at least 45%, at least 50%, at least 55%, at least 60% and/or at least 70% of a Maximum Swell Ratio for the SPH material at a time interval of 2 minutes or less. In one embodiment, the SPH material comprises a Swell Ratio Percentage in a range of from 30% to 100%, 40% to 90%, and/or 50% to 80% of a Maximum Swell Ratio at a time interval of 60 seconds or less. Furthermore, while in certain embodiments the Swelling Speed as determined by a Swell Ratio Percentage achieved at a select time interval for an SPH material may be to be relatively high, in other embodiments, the Swelling Speed may be relatively low, while still advantageously providing a relatively high Maximum Swell Ratio for the SPH body.

In addition to swelling properties of the SPH body, the ability of the SPH body to withstand forces in the intestinal environment may also be important to maintain delivery of the active agent at the target site of the intestinal tissue. In one embodiment, a Compressive Strength of the SPH body may be provided that is capable of resisting forces and/or pressures in the intestine, such as the forces caused by peristaltic waves. In one embodiment, the SPH body comprises a Compressive Strength as measured by the Yield Point of at least 5,000 Pa, such as at least 8,000 Pa, as the minimum suitable Compressive Strengths as measured by the Yield Point. According to yet further embodiments, the Compressive Strength as measured by the Yield Point may be at least 10,000 Pa, at least 15,000 Pa, at least 18,000 Pa, at least 20,000 Pa, at least 25,000 Pa, at least 30,000 Pa, at least 35,000 Pa, at least 40,000 Pa and/or at least 45,000 Pa. In certain embodiments, such as with the cationic SPH described in the Examples below, the Compressive Strength as measured by the Yield point may even be at least as high as 50,000 Pa, such as at least 60,000 Pa and/or even at least 70,000 Pa. Generally, the Compressive Strength of the SPH body as measured by the Yield Point will not exceed 100,000 Pa, and may even be less than 90,000 Pa, and/or less than 80,000 Pa. For example, in one embodiment, the Compressive Strength may be selected to be sufficiently high to survive a peristaltic wave, but not so high such that the SPH body can still be broken down by peristaltic pressured p after a predetermined amount of time, although breakdown of the SPH body can also be provided by other means. Accordingly, in one embodiment, the SPH body has a Compressive Strength as measured by the Yield Point that is in a range of from, 8,000 Pa to 100,000 Pa, such as in a range from 20,000 Pa to 90,000 Pa, and/or in a range from 30,000 Pa to 80,000 Pa.

As yet another property for enhancement of active agent delivery, the SPH body may be provided with a radial strength that is sufficient to exert a radially outward force such that the SPH body can be pressed against and/or into the vicinity of the intestinal tissue, thereby contacting and/or bringing the active agent into close proximity with the intestinal tissue. However, the Radial Swell Force generally may be selected not to exceed an amount that might cause excessive pressure or pain to a patient to whom the oral dosage form is administered. The Radial Swell Force may be measured for a surface of the SPH body that will swell to contact and/or come into proximity with the intestinal tissue, such as in the case of an elongated body 104 (e.g., a cylinder or rectangular prism), the Radial Swell Force may be measured for a surface that is along the elongated side surface 114 parallel to the longitudinal axis L of the body 104. According to one embodiment, the Radial Swell Force may be at least 15 g, at least 25 g, at least 30 g, at least 35 g, at least 40 g, at least 50 g, at least 60 g, at least 75 g, and/or at least 100 g. Generally, the Radial Swell Force will be less than 1000 g, such as less than 900 g, and even less than 800 g. For example a Radial Swell Force of the SPH body may be in the range of from 50 g to 1000 g, such as in a range of from 70 g to 250 g, and even in a range of from 75 g to 200 g.

In one embodiment, the swelling of the SPH body is such that the SPH body has a rapid rate of release from the dosage from as measured by the Capsule Escape Assay. For example, the Capsule Escape Time for the SPH body may be less than 1 minute, such as less than 45 seconds and/or less than 30 seconds.

According to one aspect, the SPH body may be formed of SPH material that exhibits properties such as those described herein, to provide an improved delivery vehicle for SPH. For example, in one embodiment, an SPH body may be formed of an ion-paired interpenetrating network SPH, in which a charged high MW structural support polymer is added to a SPH polymerizing reaction having monomers of opposite charge, which results in an ion-paired interpenetrating network (IP-IPN) with unexpectedly good physical properties that may be advantageous for intestinal delivery. It has been found that if a charged high-MW polymer additive is selected with a charge opposite to that of the polymerizing SPH matrix, a charge-paired IPN results having superior and unanticipated strength and elasticity relative to similar IPN and Semi-IPN SPH compositions without such ion-pairing.

As yet another embodiment, the SPH body can be formed of cationic SPH incorporating cationic repeat units that may provide unexpectedly good properties that are advantageous for intestinal delivery. Specifically, the cationic SPH materials can be easily made by providing cationic monomers that can be polymerized using free radical chemistry. When copolymerized as a foam along with neutral co-monomers (acrylamide, PEG-acrylate, others) and crosslinkers (methylene bisacrylamide), novel cationic SPH compositions having excellent properties can be made. Examples of suitable cationic monomers can include any one or more selected from the group consisting of 3-(amino)propyl-methacrylamide, 3-(dimethylamino)propyl-methacrylamide, 3-(trimethylammonium)propyl-methacrylamide hydrochloride, as well as substituted derivatives, copolymers and pharmaceutically acceptable salts thereof.

In one embodiment, the ion-paired IPN SPH material can be formed by incorporating a cationic a cationic structural support polymer into an anionic SPH matrix. The SPH matrix comprises anionic structural repeat units and crosslinking structural repeat units, and optionally can further comprise neutral structural repeat units, and optionally also neutral PEGylated structural repeat units. The cationic structural support polymer may be an aliphatic polymer selected from the group consisting of polyalkylacrylates, polyacrylamides, polyalkylmethacrylates, polymethacrylamides, poly-N-alkylacrylamides, poly-N-alkylmethacrylamides, substituted derivatives thereof, copolymers thereof, and pharmaceutically acceptable salts thereof. For example, the structural support polymer can be any one or more selected from the group consisting of Poly N-[3-(amino)propyl] methacrylamide, Poly N-[3-(dimethylamino)propyl] methacrylamide, Poly N-[3-(trimethylammonium)propyl] methacrylamide, Poly N-[2-(amino)ethyl] methacrylamide, Poly N-[2-(dimethylamino)ethyl] methacrylamide, Poly N-[2-(trimethylammonium)ethyl] methacrylamide, Poly [3-(amino)propyl] methacrylate, Poly [3-(dimethylamino)propyl] methacrylate, Poly [3-(trimethylammonium)propyl] methacrylate, Poly [2-(amino)ethyl] methacrylate, Poly [2-(dimethylamino)ethyl] methacrylate, Poly [2-(trimethylammonium)ethyl] methacrylate, Poly N-[2-(Diisopropylamino)ethyl] methacrylamide, Poly [2-(Diisopropylamino)ethyl] methacrylate, Poly N-[2-(Diethylamino)ethyl] methacrylamide, Poly [2-(Diethylamino)ethyl] methacrylate, Poly N-[2-(ethylpyrrolidine] methacrylamide, Poly [2-(ethylpyrrolidine] methacrylate, Poly N-[3-(amino)propyl] acrylamide, Poly N-[3-(dimethylamino)propyl] acrylamide, Poly N-[3-(trimethylammonium)propyl] acrylamide, Poly N-[2-(amino)ethyl] acrylamide, Poly N-[2-(dimethylamino)ethyl] acrylamide, Poly N-[2-(trimethylammonium)ethyl] acrylamide, Poly [3-(amino)propyl] acrylate, Poly [3-(dimethylamino)propyl] acrylate, Poly [3-(trimethylammonium) propyl] acrylate, Poly [2-(amino)ethyl] acrylate, Poly [2-(dimethylamino)ethyl] acrylate, Poly [2-(trimethylammonium)ethyl] acrylate, Poly N-[2-(Diisopropylamino)ethyl] acrylamide, Poly [2-(Diisopropylamino)ethyl] acrylate, Poly N-[2-(Diethylamino)ethyl] acrylamide, Poly [2-(Diethylamino)ethyl] acrylate, Poly N-[2-(ethylpyrrolidine] acrylamide, Poly [2-(ethylpyrrolidine] acrylate, as well as copolymers and pharmaceutically acceptable salts thereof.

According to yet another embodiment, the cationic structural support polymer can comprise a synthetic amine polymer, with suitable amine polymers (or salts thereof) including, but not limited to substituted or unsubstituted polymers or copolymers of one or more selected from the group consisting of Poly(allylamine), Poly(diallylamine), Poly(diallylmethylamine), Poly(diallyldimethyl ammonium chloride), Poly(ethyleneimine), Poly(vinylamine), Poly(l-vinylimidazole), and Poly(4-vinylpyridine), as well as copolymers and pharmaceutically acceptable salts thereof.

In another embodiment the cationic structural support polymer can comprise a cationic polymer with an INCI (International Nomenclature Cosmetic Ingredient) name designation as a “polyquaternium” compound by the Personal Care Products Council. For example: Polyquaterniums 1-47. In yet another embodiment, the cationic structural support polymer can comprise a cationic polysaccharide of natural or semi-synthetic origin. For example any selected from the group consisting of Chitosan (e.g., with degree of deacetylation from 60-99%), Trimethylammonium chitosan, Diethylaminoethyl dextran, Quaternized hydroxyethyl cellulose and derivatives, as well as all modified cationic polysaccharides and pharmaceutically acceptable salts thereof.

Furthermore, a polymeric ammonioalkyl group will further include a negatively charged counterion, such as a conjugate base of a pharmaceutically acceptable acid. Examples of suitable counterions include Cl⁻, PO₄⁻, Br⁻, CH₃SO₃⁻, HSO₄⁻, SO₄²⁻, HCO³⁻, CO₃²⁻, acetate, lactate, succinate, propionate, butyrate, ascorbate, citrate, maleate, folate, tartrate, polyacrylate, an amino acid derivative, and a nucleotide.

According to yet another embodiment, a negatively charged structural support polymer is incorporated into a cationic SPH matrix. The SPH matrix may comprise cationic structural repeat units and crosslinking structural repeat units, and may optionally comprise neutral structural repeat units, along with optional neutral PEGylated structural repeat units. The anionic structural support polymer can comprise an aliphatic polymer selected from the group consisting of polyalkylacrylates, polyacrylamides, polyalkylmethacrylates, polymethacrylamides, poly-N-alkylacrylamides, poly-N-alkylmethacrylamides, substituted derivatives thereof and copolymers thereof. For example, the anionic structural support polymer can comprise any selected from the group consisting of Poly[3-(sulfo)propyl] methacrylamide, Poly[2-(sulfo)ethyl] methacrylamide, Poly[2-carboxyethyl] methacrylate, Poly[2-methacrylamido-2-methyl-1-propanesulfonic acid, Poly[methacrylic acid] and Poly[Itaconic acid], as well as all copolymers and pharmaceutically acceptable salts thereof. Furthermore, in another embodiment the cationic SPH matrix contains an anionic polysaccharide of natural or semi-synthetic origin, such as for example any selected from the group consisting of Hyaluronic acid, Chondroitin Sulfate, Carboxymethylcellulose and Alginic acid, as well as all modified polymers and pharmaceutically acceptable salts thereof.

Ion-Paired SPH

According to one embodiment of forming a super-porous hydrogel (SPH) material, the method comprises forming a polymerization mixture by combining (i) a structural support material comprising at least one ionically charged structural support polymer having a molecular weight of at least 50,000 g/mol, the ionically charged structural support polymer having a plurality of ionically charged chemical groups, (ii) a monomer material comprising at least one ionically charged ethylenically-unsaturated monomer, and (iii) at least one cross-linking agent, forming a foam of the polymerization mixture, and polymerizing the foam to form a porous crosslinked polymeric structure having ion-pairing between a cross-linked polymer matrix formed by polymerization of the ionically charged ethylenically-unsaturated monomer with the cross-linking agent, and the ionically charged structural support polymer. Each of the ionically charged chemical groups of the ionically charged structural support polymer each have an ionic charge that is the opposite of that of a charge of the ionically charged ethylenically-unsaturated monomer.

According to yet another embodiment, a super-porous hydrogel (SPH material) can be formed according to methods described herein, which provide improved properties. According to one embodiment, the SPH material comprises a porous cross-linked polymeric structure comprising a crosslinked polymer matrix having a repeat structure of monomers comprising ionically charged chemical groups, about an ionically charged structural support polymer comprising ionically charged chemical groups, the ionically charged structural support polymer having a molecular weight of at least 50,000 g/mol. At least some of the ionically charged groups of the crosslinked polymer matrix are ion-paired with the ionically charged groups of ionically charged structural support polymer, and each of the ionically charged chemical groups of the ionically charged structural support polymer each have an ionic charge that is the opposite of that of a charge of the ionically charged chemical groups of the repeat structure of the cross-linked polymer matrix.

According to one embodiment, the SPH material comprises ionically charged chemical groups of the ethylenically-unsaturated monomer that are anionically charged, and ionically charged chemical groups of the ionically charged structural support polymer that are cationically charged. In yet another embodiment, the SPH material comprises ionically charged chemical groups of the ionically charged ethylenically-unsaturated monomer that are cationically charged, and the ionically charged chemical groups of the ionically charged structural support polymer that are anionically charged.

In one embodiment, the ionically charged ethylenically-unsaturated monomer comprises any selected from the group consisting of acrylate monomers (salts of (meth)acrylic acid), salts of esters of (meth) acrylic acid, salts of N-alkyl amides of (meth)acrylic acid, sulfopropyl acrylate monomers, PEG acrylate, and 2-(acryloyloxy)ethyl trimethylammonium methyl sulfate, and/or salts thereof. In yet another embodiment, the monomer material further comprises non-ionically charged ethylenically-unsaturated monomers, including any selected from the group consisting of acrylamide monomers, acrylamidopropyl monomers, esters of (meth)acrylic acid and their derivatives (2-hydroxyethyl (meth) acrylate, hydroxypropyl(meth) acrylate, butanediol monoacrylate), N-alkyl amides of (meth) acrylic acid, N-vinyl pyrrolidone, (meth)acrylamide derivatives (N-isopropyl acrylamide, N-cyclopropyl (meth)acrylamide, N,N-dimethylaminoethyl acrylate, and 2-acrylamido-2-methyl-1-propanesulfonic acid, and/or salts thereof.

In one embodiment, the monomer material further comprises an acrylate monomer having a polyethylene glycol repeat group of the following formula:

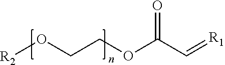

where R₁ and R₂ are each independently hydrocarbyl with 6 carbons or less, or hydrogen, n is on average in a range of from 2 to about 20, or is in a range of from about 5 to about 15, and/or is in a range of from about 8 to 12. For example, in embodiment, the monomer material comprises MPEG acrylate (480).

According to yet another embodiment, the structural support polymer can comprise any of the cationic and/or anionic support polymers described above. Further structural support materials can include any selected from the group consisting of a polysaccharide, chitosan, chitins, alginate, cellulose, cyclodextrin, dextran, gums, lignins, pectins, saponins, deoxyribonucleic acid, ribonucleic acids, polypeptides, protein, albumin, bovine serum albumin, casein, collagen, fibrinogen, gelatin, gliaden, poly amino acids, synthetic polymers, (meth) acrylamide polymer, (meth)acrylic acid polymer, (meth) acrylate polymer, acrylonitrile, ethylene polymers, ethylene glycol polymers, ethyleneimine polymers, ethyleneoxide polymers, styrene sulfonate polymers, vinyl acetate polymers, vinyl alcohol polymers, vinyl chloride polymers, vinylpyrrolidone polymers and/or derivatives, salts, and/or homo or copolymers thereof. Furthermore, in one embodiment, the ionically charged structural support polymer comprises a molecular weight of at least 55,000 g/mol MW, at least 65,000 g/mol MW, at least 80,000 g/mol MW, at least 100,000 g/mol MW, at least 125,000 g/mol MW, at least 150,000 g/mol MW, at least 175,000 g/mol MW, at least 200,000 g/mol MW, and/or at least 225,000 g/mol MW. Generally, a molecular weight of the ionically charged structural polymer will not exceed 1,000,000 g/mol MW. For example, a molecular weight of the ionically charged structural polymer may be in the range of from 50,000 g/mol MW to 250,000 g/mol MW.

In embodiments herein, the cross-linking agent may be capable of cross-linking together polymer chains generated from the polymerization of the monomer material to form the SPH matrix. Further, in certain embodiments, the ionically charged structural support polymer, while it may be ion-paired with the SPH matrix in the final SPH polymeric structure, may not itself be further crosslinked, either with itself or with moieties in the SPH matrix. That is, the ionically charged structural support polymer may be one that is not reactive with and/or cross-linkable via the cross-linking agent provided to link together polymeric chains generated by polymerization of the monomer material, such that the SPH polymeric structure comprises a crosslinked polymer matrix (e.g., formed from polymerization of the monomers in the presence of the cross-linking agent), that may be ion-paired with, but is not otherwise covalently cross-linked to, the ionically charged structural support polymer, and the ionically charged structural support polymer is not further cross-linked with itself or another moiety. For example, in some embodiments, the SPH material can comprise a semi-interpenetrating network, where the SPH matrix formed from the polymerization of the monomer material (e.g., the ionically charged ethylenically unsaturated monomers) is cross-linked to form a matrix about the ionically charged structural support polymer, but the ionically charged structural support polymer is not itself further cross-linked. Furthermore, in one embodiment, any cross-linking agent provided to cross-link the polymerization mixture comprises at least 50 wt %, at least 65 wt %, at least 75 wt %, at least 85 wt %, at least 90 wt %, at least 95 wt %, and/or even 100% by weight of an ethylenically unsaturated cross-linking monomer. That is, any cross-linking agent provided as a part of the SPH formation process, and/or incorporated into the SPH polymeric structure, is predominantly and even entirely one that cross-links via formation of covalent bonds using the ethylenically unsaturated group. Furthermore, in one embodiment, any cross-linking agent provided to cross-link the polymerization mixture comprises at least 50 wt %, at least 65 wt %, at least 75 wt %, at least 85 wt %, at least 90 wt %, at least 95 wt %, and/or even 100% by weight of a cross-linking agent that is capable of forming covalent bonds with the monomer material and/or polymeric chains generated therefrom, but is not reactive to form bonds with the ionically charged structural support polymer, either covalently or ionically.

In one embodiment, the cross-linking agent comprises an ethylenically unsaturated cross-linking monomer comprising any selected from the group consisting of N,N′-methylene bisacrylamide, N,N′-ethylene bisacrylamide, (poly)ethylene glycol di(meth)acrylate, ethylene glycol diglycidyl ether, glycidyl methacrylate, polyamidoamine epichlorohydrin resin, trimethylolpropance triacrylate (TMPTA), piperazine diacrylamide, glutaraldehyde, epicholorhydrin, and N,N′-diallyltartardiamide, as well as substituted derivatives, copolymers and pharmaceutically acceptable salts thereof.

The polymerization can be initiated using mechanisms including photochemical, thermal, chemical, etc., such as via the use of initiators such as ammonium persulfate (APS), tetraethylenediamine (TEMED), and others. The foaming of the polymerization mixture can be provided via various techniques, such as by including a foaming or blowing agent in the polymerization mixture, including for example sodium bicarbonate and/or ammonium bicarbonate, which can be mixed with an acid to generated carbon dioxide gas.

In one embodiment, the polymerization mixture that is polymerized to form the SPH material comprises at least 1% by weight, at least 5% by weight, at least 8% by weight, and/or at least 10% by weight of the monomer material comprising at the least one ionically charged ethylenically-unsaturated monomer, and no more than 35% by weight, 25% by weight, 18% by weight and/or 15% by weight of the monomer material comprising at the least one ionically charged ethylenically-unsaturated monomer, such as for example acrylic acid, and/or a salt thereof. In another embodiment, the SPH material comprises at least 0.25%, at least 0.3% by weight, at least 0.45% by weight, and/or at least 0.5% by weight of the structural support material comprising the at least one ionically charged structural support polymer, and no more than 1% by weight, no more than 0.90% by weight, no more than 0.85% by weight and/or no more than 0.75% by weight of the structural support material comprising the at least one ionically charged structural support polymer, such as chitosan and/or a salt thereof. In another embodiment, the polymerization mixture that is polymerized to form the SPH material comprises at least 0.001% by weight, at least 0.01% by weight, at least 0.1% by weight, and/or at least 0.5% by weight of the cross-linking agent, and no more than 1% by weight, 0.8% by weight, 0.7% by weight and/or 6% by weight of the cross-linking agent, such as methylene bisacrylamide. In another embodiment, the polymerization mixture that is polymerized to form the SPH material comprises at least 1% by weight, at least 5% by weight, at least 15% by weight, and/or at least 25% by weight of a non-ionically charged ethylenically unsaturated monomer, and no more than 50% by weight, 45% by weight, 35% by weight and/or 30% by weight of the non-ionically charged ethylenically unsaturated monomer, such as acrylamide. In yet another embodiment, the polymerization mixture that is polymerized to form the SPH material comprises at least 1% by weight, at least 5% by weight, at least 8% by weight, and/or at least 10% by weight of an acrylate monomer having a polyethylene glycol repeat group, and no more than 35% by weight, 30% by weight, 20% by weight and/or 15% by weight of the acrylate monomer having a polyethylene glycol repeat group, such as MPEG acrylate.

According to yet another embodiment, the amount of “solid” material (e.g., non-liquid) provided in the polymerization mixture may be maintained at a relatively high proportion of the polymerization mixture, to provide improved properties. For example, according to one embodiment, the polymerization mixture that is polymerized to form the SPH material can comprise a combined amount of the monomer material, structural support material, and at least one cross-linking agent, that is greater than 25%, 30%, 35%, 40% and/or 50% by weight of the total weight of the polymerization mixture, and no more than 90%, no more than 80% and/or no more than 75% by weight of the total weight of the polymerization mixture.

Cationic SPH

According to one embodiment of a method of forming a super-porous hydrogel (SPH) material, the method comprises forming a polymerization mixture by combining (i) a monomer material comprising at least one cationically charged ethylenically-unsaturated monomer, and optionally at least one non-ionically charged ethylenically unsaturated monomer, and (ii) at least one cross-linking agent, forming a foam of the polymerization mixture, and polymerizing the foam to form a porous crosslinked polymeric structure formed by polymerization of the cationically charged ethylenically-unsaturated monomer with the cross-linking agent, and optionally with the neutral ethylenically unsaturated monomer. The porous crosslinked polymeric structure formed with the cationically charged monomer comprises a Swelling Ratio of at least 25, and a Compressive Strength as measured by the Yield Point of at least 5000 Pascals.

According to yet another embodiment, a super-porous hydrogel (SPH) material can be provided that comprises a porous cross-linked polymeric structure comprising a crosslinked polymer matrix having a repeat structure of monomer residues obtained from cationically charged ethylenically-unsaturated monomers, and optionally monomer residues obtained from non-ionically charged ethylenically-unsaturated monomers. The porous cross-linked polymeric structure formed from the cationically charged monomer comprises a Swelling Ratio of at least 25, and a Compressive Strength as measured by the Yield Point of at least 5000 Pascals.

In one embodiment, the cationically charged ethylenically-unsaturated monomer comprises any selected from the group consisting of 3-(amino)propyl methacrylamide, 3-(dimethylamino)propyle-methacrylamide, 3-(trimethylammonium)propyl-methacrylamide, and/or salts thereof. In another embodiment, the SPH material comprises non-ionically charged ethylenically-unsaturated monomers, including any selected from the group consisting of acrylamide monomers, acrylamidopropyl monomers, esters of (meth)acrylic acid and their derivatives (2-hydroxyethyl (meth) acrylate, hydroxypropyl(meth) acrylate, butanediol monoacrylate), N-alkyl amides of (meth) acrylic acid, N-vinyl pyrrolidone, (meth)acrylamide derivatives (N-isopropyl acrylamide, N-cyclopropyl (meth)acrylamide, N.N-dimethylaminoethyl acrylate, and 2-acrylamido-2-methyl-1-propanesulfonic acid and/or salts thereof. According to yet another embodiment, the monomer material further comprises an acrylate monomer having a polyethylene glycol repeat group of the following formula:

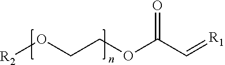

where R₁ and R₂ are each independently hydrocarbyl with 6 carbons or less, or hydrogen, n is on average in a range of from 2 to about 20, or is in a range of from about 5 to about 15, and/or is in a range of from about 8 to 12. For example, the monomer material can comprise MPEG acrylate (408).

Furthermore, in one embodiment, the crosslinking agent comprises any of those specified elsewhere herein, such at least one selected from the group consisting of N,N′-methylene bisacrylamide, N,N′-methylene bisacrylamide, (poly)ethylene glycol di(meth)acrylate, ethylene glycol diglycidyl ether, glycidyl methacrylate, polyamidoamine epichlorohydrin, and N,N′-diallyltartardiamide, as well as substituted derivatives, copolymers and pharmaceutically acceptable salts thereof.

In one embodiment, the polymerization mixture that is polymerized to form the SPH material comprises at least 1% by weight, at least 5% by weight, at least 8% by weight, and/or at least 10% by weight of the monomer material comprising at least one cationically charged ethylenically-unsaturated monomer, and no more than 35% by weight, 30% by weight, 25% by weight and/or 20% by weight of the monomer material comprising at least one cationically charged ethylenically-unsaturated monomer, such as (3-acrylamidopropyl)trimethylammonium, and/or a salt thereof.

In another embodiment, the polymerization mixture that is polymerized to form the SPH material comprises at least 0.001% by weight, at least 0.01% by weight, at least 0.1% by weight, and/or at least 0.5% by weight of the cross-linking agent, and no more than 1% by weight, 0.8% by weight, 0.7% by weight and/or 6% by weight of the cross-linking agent, such as methylene bisacrylamide. In another embodiment, the polymerization mixture that is polymerized to form the SPH material comprises at least 1% by weight, at least 5% by weight, at least 15% by weight, and/or at least 25% by weight of a non-ionically charged ethylenically unsaturated monomer, and no more than 50% by weight, 45% by weight, 35% by weight and/or 30% by weight of the non-ionically charged ethylenically unsaturated monomer, such as acrylamide. In yet another embodiment, the polymerization mixture that is polymerized to form the SPH material comprises at least 1% by weight, at least 5% by weight, at least 8% by weight, and/or at least 10% by weight of an acrylate monomer having a polyethylene glycol repeat group, and no more than 35% by weight, 30% by weight, 20% by weight and/or 15% by weight of the acrylate monomer having a polyethylene glycol repeat group, such as MPEG acrylate.

In one embodiment, the polymerization mixture that is polymerized to form the SPH material comprises a combined amount of the monomer material and at least one cross-linking agent, that is greater than 25%, 30%, 35%, 40% and/or 50% by weight of the total weight of the polymerization mixture, and no more than 90%, no more than 80% and/or no more than 75% by weight of the total weight of the polymerization mixture.

According to yet another embodiment, the SPH material, such as that formed by any of the processes described herein, maybe at least partially dried in a humidified environment comprising an environmental humidity of at least 50%, at least 65%, and/or at least 75%. For example, the SPH material may be dried under conditions such that at least some moisture is retained in the SPH material, such as to provide an amount of retained water of at least 2.5%, at least 5%, at least 8%, but no more 10% by weight of the SPH material, to form Compressible SPH. The SPH material that at least partly retains moisture may be more elastic and so may be compressible into a predetermined shape when preparing the SPH material for incorporation into the dosage form, such as compressible into a selected size of capsule (e.g., size 000 capsule). In one embodiment, a dried SPH material having too little moisture content may be re-humidified to have the amount of retained water described herein.

According to one embodiment, the SPH material that retains some moisture (Compressible SPH) may be sufficient compressible and/or elastic such that a volume of the SPH material is compressible to a Compressed State having a compressed volume corresponding to less than 90%, less than 80%, less than 75%, less than 60% and/or less than 50% of the SPH material in the Uncompressed State. Furthermore, the SPH material may be compressed into the Compressed State while retaining a Swell Speed in which a Swell Ratio Percentage of at least 30%, at least 35%, at least 45%, at least 50%, at least 55%, at least 60% and/or at least 70% of a Maximum Swell Ratio for the SPH material is achieved at a time interval of 60 seconds or less. According to one embodiment, the SPH material in the Compressed State exhibits a Volume Swell Ratio of at least 20, at least 30, at least 40, at least 50, at least 60, at least 70 and/or at least 80. In contrast, the SPH material in the Uncompressed State may exhibit a Volume Swell Ratio of at least 2, at least 4, at least 5, at least 8 and/or at least 10. That is, the SPH material in the Compressed State may exhibit a Volume Swell Ratio that is at least 2 times, at least 3 times, at least 4 times and/or at least 5 times a Volume Swell Ratio of the SPH material in an Uncompressed State. Accordingly, in certain embodiments, SPH may be provided in a Compressed State in the dosage form, as the higher Volume Swell Ratio of the Compressed SPH may facilitate incorporation into a relatively smaller dosage form, while still allowing for sufficient swell characteristics when deployed in the gastrointestinal environment.

Permeation Enhancer

In yet another embodiment, the oral dosage form comprises at least one permeation enhancer to enhance permeation of the active agent through the intestinal tissue. In some embodiments, the permeation enhancer may be capable of opening a tight junction between cells (e.g., intestinal cells or epithelial cells). A permeation enhancer may, in some instances, facilitate uptake of an agent into epithelial cells. Representative classes of permeation enhancers include, but are not limited to, a fatty acid, a medium chain glyceride, a surfactant, a steroidal detergent, an acyl carnitine, lauroyl carnitine, palmitoyl carnitine, an alkanoyl choline, an N-acetylated amino acid, esters, salts, bile salts, sodium salts, nitrogen-containing rings, derivatives thereof, and combinations thereof. The permeation enhancer may be anionic, cationic, zwitterionic, or nonionic. Anionic permeation enhancers include, but are not limited to, sodium lauryl sulfate, sodium decyl sulfate, sodium octyl sulfate, N-lauryl sarcosinate, and sodium carparate. Cationic permeation enhancers include, but are not limited to, cetyltrimethyl ammonium bromide, decyltrimethyl ammonium bromide, benzyldimethyldodecyl ammonium chloride, myristyltrimethylammonium chloride, and dodecylpyridinium chloride. Zwitterionic permeation enhancers include, but are not limited to, N-dodecyl-N,N-dimethyl-3-ammonio-1-propanesulfonate, 3-(N,N-dimethylpalmitylammonio)propanesulfonate. Fatty acids include, but are not limited to, butyric, caproic, caprylic, pelargonic, capric, lauric, myristic, palmitic, stearic, arachidic, oleic, linoleic, and linolinic acid, salts thereof, derivatives thereof, and combinations thereof. In some embodiments, a fatty acid may be modified as an ester, for example, a glyceride, a monoglyceride, a diglyceride, or a triglyceride. Bile acids or salts including conjugated or unconjugated bile acid permeation enhancers include, but are not limited to, cholate, deoxycholate, tauro-cholate, glycocholate, taurodexycholate, ursodeoxycholate, tau roursodeoxycholate, chenodeoxycholate, derivates thereof, salts thereof, and combinations thereof. In some embodiments, permeation enhancers include a metal chelator, such as EDTA or EGTA, a surfactant such as sodium dodecyl sulfate, polyethylene ethers or esters, polyethylene glycol-12 lauryl ether, salicylate polysorbate 80, nonylphenoxypolyoxyethylene, dioctyl sodium sulfosuccinate, saponin, palmitoyl carnitine, lauroyl-l-carnitine, dodecyl maltoside, acyl carnitines, alkanoyl cjolline, and combinations thereof. Other permeation enhancers include, but are not limited to, 3-nitrobenzoate, zoonula occulden toxin, fatty acid ester of lactic acid salts, glycyrrhizic acid salt, hydroxyl beta-cyclodextrin, N-acetylated amino acids such as sodium N-[8-(2-hydroxybenzoyl)amino]caprylate and chitosan, micelle forming agents, passageway forming agents, agents that modify the micelle forming agent, agents that modify the passageway forming agents, salts thereof, derivatives thereof, and combinations thereof. In some embodiments, micelle forming agents include bile salts. In some embodiments, passageway forming agents include antimicrobial peptides. In some embodiments, agents that modify the micelle forming agents include agents that change the critical micelle concentration of the micelle forming agents. An exemplary permeation enhancer is 1% by weight 3-(N,N-dimethylpalmitylammonio)propanesulfonate. Permeation enhancers are also described in patent application publication US 2013/0274352, the contents of which are incorporated in their entirety herein. In one embodiment, the permeation enhancers can comprise at least one of EDTA, palmitoyl carnitine, lauroyl carnitine, dimethyl palmitoyl ammonio propanesulfonate (PPS), and sodium caprate.

In one embodiment, permeation enhancers selected for the oral dosage form may be selected on the basis of one or more of the predominant permeation mechanism and the hydrophilicity and/or hydrophobicity of the permeation enhancer. For example, permeation enhancers that are fatty esters and/or permeation enhancers having nitrogen-containing rings may exhibit more paracellular transport activity, whereas cationic and zwitterionic permeation enhancers may exhibit more transcellular activity, as described for example in the article to Whitehead and Mitragotri entitled “Mechanistic Analysis of Chemical Permeation Enhancers for Oral Drug Delivery” in Pharmaceutical Research, Vol. 25, No. 6, June 2008, pages 1412-1419, which is hereby incorporated by reference herein in its entirety. Furthermore, for those permeation enhancers having a transcellular mechanism, increases in hydrophobicity of the permeation enhancer may enhance this mechanism, whereas for permeation enhancers having more paracellular transport activity, greater enhancement may be seen for those permeation enhancers that are more hydrophillic (such as by interacting with hydrophilic constituents of tight junctions). In one embodiment the relative hydrophobicity/hydrophilicity of the enhancer may be determined by its log P value, with P being the octanol/water partition coefficient for the compound. For example, in one embodiment, to enhance transcellular transport, a permeation enhancer may have a log P value of at least 2, such as at least 4, and even at least 6. Conversely, to enhance paracellular transport, a permeation enhancer may in one embodiment have a log P of less than about 4, such as less than 2, and even less than 0.

A content of the permeation enhancer in the oral dosage form in one embodiment may be at least about 0.01% by weight, such as at least about 0.1% by weight, and no more than about 80% by weight, and may even be less than about 30% by weight. For example, in one embodiment, the content of permeation enhancer in the oral dosage form may be at least about 0.01% by weight, such as at least about 0.1% by weight, including at least about 1% by weight, such as at least about 5% by weight, and even at least about 10% by weight, such as at least about 30% by weight, or even at least about 50% by weight, such as at least about 70% by weight. For example, in one embodiment, the content of permeation enhancer may be in the range of from 0.1% by weight to 70% by weight, such as from about 0.1% by weight to about 20% by weight, and even from about 1% by weight to about 10% by weight.

Furthermore, according to certain embodiments, the one or more permeation enhancers may be provided at one or more of the active agent delivery regions 106 at the exterior surface 108 of the SPH body 104. In one embodiment, a significant fraction of the total amount of permeation enhancer provided in the dosage form is contained at the one or more active agent delivery regions at the exterior surface 108 of the SPH body 104. For example, the one or more active agent delivery regions at the exterior surface of the monolithic body comprise at least about 50%, at least 60%, at least 70%, at least 80%, at least 90%, at least 95%, at least 98% and/or at least 99% of the permeation enhancer contained in the dosage form.

In one embodiment, a total amount of permeation enhancer provided in the dosage form may be reduced, as the permeation enhancer provided at the exterior surface may be brought into close relationship with the target tissue site by virtue of swelling of the SPH body, thereby allowing for less permeation enhancer to provide a same permeating effect. In one embodiment, the permeation enhancer may be provided in a total dosage amount that is in the range of from 0.1 mg to 800 mg per dosage form, such as 0.1 mg to 600 mg per total dosage form, such as a dosage in the range of from 1 mg to 200 mg, and even in a dosage in the range of from 10 mg to 40 mg per total dosage form. In one embodiment, the permeation enhancer is provided in a range of at least 5 mg to no more than 50 mg per dosage form, such as at least 15 mg to no more than 35 mg per dosage form. In another embodiment, the permeation enhancer is provided in a range of at least 50 mg to no more than 200 mg per dosage for, such as at least 75 mg to no more than 100 mg per dosage form. For example, the dosage form may have the permeation enhancer in a content of at least 0.1 mg per dosage form, such as at least 1 mg per dosage form, and even at least 10 mg per dosage form, such as at least 30 mg per dosage form, at least 50 mg per dosage form, and even larger values such as at least 100 mg per dosage form, at least 200 mg per dosage form, at least 400 mg per dosage form, and at least 600 mg per dosage form. In one embodiment, the dosage of the permeation enhancer will not exceed 600 mg for the dosage form, and may even be less than 400 mg, such as less than 200 mg, and even less than 100 mg, such as less than 50 mg, and even less than 30 mg. In one embodiment, a permeation enhancer comprising sodium caprate is provided in an amount of at least 10 mg and no more than 50 mg per dosage form. In another embodiment, a permeation enhancer comprising PPS is provided in an amount of at least 10 mg and no more than 50 mg per dosage form.

Other Additives

The oral dosage form can comprise further additives in addition to the active agent, SPH composition and optional permeation enhancer.

For example, in one embodiment, the dosage form can comprise a gelling agent capable of is capable of forming a gel upon exposure to an intestinal environment. In particular, in one embodiment, the gelling agent is exposed to intestinal fluids upon dissolution of a protective coating or other outer layer, thereby causing the gelling agent to thicken and form a viscous gel material. Without being limited to any particular theory, it is believed that including the gelling agent in the oral dosage form can improve delivery of the active agent by forming a thickened and semi-coherent mass with the active agent upon exposure to the intestinal environment. The gelling agent may thus, in certain embodiments, improve delivery of an active, as well as improve retention of the active agent adjacent intestinal tissue. The gelling agent according to one embodiment comprises an agent that is capable of providing a gelling and/or thickening effect to a liquid, such as in an intestinal fluid. Suitable gelling agents can include at least one of pectin, hydroxypropylmethylcellulose (HPMC), acrylic acid polymer and copolymers, including carbopol polymers (such as CARBOPOL 934 P), acacia, alginic acid, polyvinyl alcohol, sodium alginate, tragacanth, methylcellulose, poloxamers, carboxymethyl cellulose, and ethyl cellulose. In one embodiment, the gelling agent comprises at least one of pectin, HPMC, and a carbopol polymer (e.g., CARBOPOL 934 P). Furthermore, in one embodiment a component that acts in concert with the gelling agent can be provided with the gelling agent to enhance gel formation. For example, in a case where pectin is used as a gelling agent, sucrose may also be provided to enhance gel formation by the pectin gelling agent. Other components that assist in gel formation, such as for example at least one of sucrose, mannitol, and fructose, may also be provided in combination with pectin or other gelling agent to provide for gel formation.

A content of the gelling agent in the oral dosage form in one embodiment can be selected according to the extent of gelling and/or thickening to be provided, as well as the structure and configuration of the oral dosage form. In one embodiment, the oral dosage form has at least about 1% by weight of a gelling agent. By way of further example, in one embodiment the oral dosage form has at least about 5% by weight of a gelling agent. By way of further example, in one embodiment the oral dosage form has at least about 10% by weight of a gelling agent. By way of further example, in one embodiment the oral dosage form has at least about 30% by weight of a gelling agent. In general, the content of the gelling agent in the oral dosage form will be less than about 50% by weight. By way of further example, in one embodiment the oral dosage form has a content of the gelling agent of less than 30% by weight. By way of further example, in one embodiment the oral dosage form has a content of the gelling agent of less than 10% by weight. For example, a content of gelling agent in the oral dosage form may be from about 1% by weight to about 50% by weight, such as from about 5% by weight to about 25% by weight, and even about 10% by weight to about 20% by weight. Furthermore, in one embodiment the oral dosage form is substantially absent any gelling agent, and thus may have an amount of gelling agent that is less than about 1% by weight, such as zero gelling agent in the composition.

In another embodiment, the oral dosage form may comprise an osmagent that assists in delivery of the active agent. Without being limited by any one theory, it is believed that the osmagent may assist in expelling the active agent from the oral dosage form, by absorbing water and pushing the active agent from the oral dosage form, and/or may help to open tight junctions in the intestine by pulling water therefrom. In one embodiment, an osmagent capable of being hydrated may include water-soluble salts, carbohydrates, small molecules, amino acids, water-soluble hydrogel forming polymers, and combinations thereof. Exemplary water-soluble salts may include, without limitation, magnesium chloride, magnesium sulfate, lithium chloride, sodium chloride, potassium chloride, lithium sulfate, sodium sulfate, potassium sulfate, sodium hydrogen phosphate, potassium hydrogen phosphate, sodium acetate, potassium acetate, magnesium succinate, sodium benzoate, sodium citrate, sodium ascorbate, and the like, and combinations thereof. Exemplary carbohydrates may include sugars such as arabinose, ribose, xylose, glucose, fructose, galactose, mannose, sucrose, maltose, lactose, raffinose, and the like, and combinations thereof. Exemplary amino acids may include glycine, leucine, alanine, methionine, and the like, and combinations thereof. Exemplary water-soluble hydrogel forming polymers may include sodium carboxy methylcellulose, hydroxypropyl methylcellulose (HPMC), hydroxyethyl methylcellulose, crosslinked PVP, polyethylene oxide, carbopols, polyacrylamindes, and the like, and combinations thereof. In one embodiment, the osmagent provided in the oral dosage form comprises at least one of sucrose, mannitol, fructose and polyethylene glycol. A content of the osmagent in the oral dosage form in one embodiment may be at least about 1% by weight, and less than about 60% by weight, such as from about 10% by weight to about 50% by weight, and even from about 20% by weight to about 40% by weight.

In one embodiment, the oral dosage form can comprise one or more controlled release/extended release agents, typically in the form of a polymeric material that is capable of forming a matrix about the active agent upon exposure to fluid, to slow release of the active agent from the dosage form. For example, the dosage form can comprise one or more the gelling agents described above as a controlled release/extended release agent. For example, the controlled release/extended release agent can comprise one or more of pectin, hydroxypropylmethylcellulose (HPMC), acrylic acid polymer and copolymers, including carbopol polymers (such as CARBOPOL 934 P), acacia, alginic acid, polyvinyl alcohol, sodium alginate, tragacanth, methylcellulose, poloxamers, carboxymethyl cellulose, and ethyl cellulose. In one embodiment, the controlled release/extended release agent comprises hydroxypropyl methyl cellulose (HPMC) as a controlled release/extended release agent. The controlled release/extended release agent can be incorporated into one or more active agent regions 105 of the dosage form that contain the at least one active agent, such as for example in either tablet or capsule form.

Other additives and/or excipients that can be provided as a part of the oral dosage form can include one or more of stabilizers, glidants, bulking agents, anti-adherents, mucoadhesive agents, binders, sorbents, preservatives, cryoprotectants, hydrating agents, enzyme inhibitors, mucus modifying agents (e.g., mucus drying agents, etc.), pH modifying agents, solubilizers, plasticizers, crystallization inhibitors, bulk filling agents, bioavailability enhancers, and combinations thereof. In some embodiments, the additives and/or excipients may include polyethylene glycols, polyethylene oxides, humectants, vegetable oils, medium chain mono, di-, and triglycerides, lecithin, waxes, hydrogenated vegetable oils, colloidal silicon dioxide, polyvinylpyrrolidone (PVP) (“povidone”), celluloses, CARBOPOL® polymers (Lubrizol Advanced Materials, Inc.) (i.e., crosslinked acrylic acid-based polymers), acrylate polymers, pectin, sugars, magnesium sulfate, or other hydrogel forming polymers. For example, in an embodiment where compressed tablets are formed for providing to the exterior surface of any SPH body, the compressed tablets may contain binders and other materials typically provided to aid in tablet formation, and additives may also be incorporated in other configurations according to the structure of the dosage form to be provided.

Protective Coating

The oral dosage form according to one embodiment further comprises a protective coating that at least partially protects the oral dosage form from the acidic environment in the stomach to deliver the active agent to a region of the intestine. The protective coating can, in one embodiment, form an outer coating of the oral dosage form that protects the active agent and/or SPH, or other additives inside the oral dosage form. While in one embodiment the protective coating completely covers an outer surface of the delivery structure comprising the SPH body and active agent of the dosage form, the protective coating may also optionally be devised to cover only a portion of the outer surface of the delivery structure. The protective coating can also comprise only a single coating layer, or can be configured as multiple coating layers.

According to one embodiment, the protective coating may be an enteric coating that is a pH dependent coating, having an enteric material that is a polymer that is substantially insoluble in the acidic environment of the stomach, but that has increased solubility in intestinal fluids that are at a higher pH. That is, the enteric coating may preferentially dissolve and/or become at least partially permeable in the intestine as opposed to in the stomach. For example, the enteric coating may be formed of an enteric material that is substantially insoluble at a pH below about 5, such as in the acidic environment of the stomach, but that becomes soluble at higher pH, such as a pH of at least about 5.5 for the duodenum, a pH of at least about 6.5 for the jejunum, and a pH of at least about 7.0, such as at least about 7.5 for the ileum (the duodenum, jejunum and ileum are part of the small intestine). That is, the enteric coating can be selected to be insoluble at lower pH, but soluble at a higher pH, such that the enteric coating can be made to dissolve and/or become at least partially permeable and release the contents of the oral dosage form once an environment of the gastrointestinal system is reached having a pH in which the material of the enteric coating is soluble. Accordingly, suitable enteric materials for forming the enteric coating in one embodiment are those that are not soluble until a pH of at least about 5.5 is reached, such as a pH of at least about 6.0. In one embodiment, suitable enteric materials for forming the enteric coating in one embodiment are those that are not soluble until a pH of at least about 6.5 is reached, such as a pH of at least about 7.0, and even a pH of at least about 7.5. Exemplary enteric materials include cellulose acetate phthalate (CAP), hydroxypropyl methylcellulose phthalate (HPMCP), polyvinyl acetate phthalate (PVAP), hydroxypropyl methylcellulose acetate succinate (HPMCAS), cellulose acetate trimellitate, hydroxypropyl methylcellulose succinate, cellulose acetate succinate, cellulose acetate hexahydrophthalate, cellulose propionate phthalate, cellulose acetate maleate, cellulose acetate butyrate, cellulose acetate propionate, copolymer of methylmethacrylic acid and methyl methacrylate, copolymer of methyl acrylate, methylmethacrylate and methacrylic acid, copolymer of methylvinyl ether and maleic anhydride (Gantrez ES series), ethyl methyacrylate-methylmethacrylate-chlorotrimethylammonium ethyl acrylate copolymer, poly(vinylalcohol), natural resins such as zein, shellac and copal collophorium, and several commercially available enteric dispersion systems (e.g., Eudragit L30D55, Eudragit FS30D, Eudragit L100, Eudragit S100, Kollicoat EMM30D, Estacryl 30D, Coateric, Kollicoat MAE 100P and Aquateric). For example, in one embodiment the enteric materials used to form the enteric coating can comprise at least one of Eudragit S100 (poly(methacrylic acid-co-methyl methacrylate) 1:2), Eudragit L100 (poly(methacrylic acid-co-methyl methacrylate) 1:1), and Kollicoat MAE 100P (methacrylic acid ethyl acrylate copolymer 1:1). The solubility of each of the above materials at a specific pH is either known or is readily determinable in vitro. For example, the foregoing is a list of possible materials, but one of skill in the art with the benefit of the instant disclosure would recognize that the foregoing list is not comprehensive and that there are other enteric materials that may be used. In yet another embodiment, the protective coating may be one that dissolved and/or becomes partially permeable due to a change in environment that is unrelated to pH. Furthermore, in another embodiment, the protective and/or enteric coating may be one that dissolves and/or becomes at least partially permeable at a predetermined rate as it passes through the gastrointestinal system, to provide a controlled and/or timed release of the active agent at a predetermined region of the intestine.

In one embodiment, the protective coating comprises at least a portion thereof that becomes permeable and/or dissolves under predetermined conditions, such as at a predetermined pH (e.g., a pH at a targeted site of the intestine), or following exposure to fluid for a pre-determined period of time (e.g., controlled release following administration at a predetermined point in time). In one embodiment, the protective coating substantially entirely comprises a coating of a material that becomes permeable and/or dissolved under the predetermined conditions.

According to yet another embodiment, the protective coating can comprise a first coating region that becomes permeable and/or dissolved under predetermined conditions, and a second coating region that substantially does not become permeable and/or does not dissolve under the predetermined conditions, and/or that becomes permeable and/or dissolves to a lesser extent than the first coating region. Such first and second coating regions may be provided, for example, in embodiments where different regions of the dosage form are to be released at different points in time and/or at different rates. For example, a first coating region may be provided to at least partially coat a section of the dosage form that covers one or more active agent regions on the exterior surface of the SPH body, whereas as second coating region may be provided to at least partially coat a section of the dosage form containing the SPH body but not containing any of the active agent regions, but covering a portion of the SPH body, to provide exposure rates of portions of the dosage form having the active agent regions versus those without active agent regions. In yet another embodiment, the protective coating comprises the first coating region that becomes permeable and/or dissolves under the predetermined conditions, as a major portion of the protective coating. For example, first coating region may be provided as a part of the protective coating such that it covers at least 25% and even at least 35% of the surface of the dosage form, such as at least 40%, and even at least 50%, such as at least 60% and even 75%, such as at least 90% of the surface of the dosage form. In yet another embodiment, the first coating region that becomes at least partially permeable and/or dissolves under the predetermined conditions may cover at least 25% and even at least 35% of a surface of a region of the oral dosage form containing the active agent delivery region(s), such as at least 40% and even at least 50%, include at least 60% and even at least 75%, such as at least 90% of the surface of the region.

In one embodiment, by providing a protective coating having a permeable and/or dissolving portion that surrounds a majority of the surface of the dosage form, the contents of the dosage form can be effectively released, and in a multi-directional manner, without unnecessarily retaining contents inside the dosage form. Furthermore, in yet another embodiment, by providing the permeable and/or dissolving portion about a majority of at least the surface of a region of the dosage form containing the active agent delivery region(s), good release of the SPH body and active agent delivery regions from a relatively large surface region of the dosage form can be provided.

The protective coating is formed on the surface of the delivery structure according to a suitable method. In one embodiment, the protective coating is formed by spray coating materials such as enteric materials onto the surface of the delivery structure, until a coating having a thickness within a predetermined range has been formed. The protective material may, in one embodiment, be sprayed relatively uniformly on the delivery structure to provide a protective coating having a uniform thickness on the surface of the oral dosage form. The protective coating may also, in another embodiment, be sprayed non-uniformly, according to a configuration of the oral dosage form and the desired release characteristics. In yet another embodiment, the protective coating can be formed on the surface of the delivery structure by a dip-coating method, where the surface of the oral dosage form is dipped or otherwise immersed in a fluid containing the protective coating materials, such as enteric coating materials, to form a coating of the protective materials on the surface.

In some embodiments, the oral dosage form may be configured for controlled release of the active agent at a region in the intestine, for example by providing a protective coating corresponding to an enteric coating that provides for controlled release at a predetermined pH and/or pH range. Additionally and/or alternatively, other ingredients and/or excipients may be provided in the oral dosage form to provide for a controlled release of the active agent and SPH body. In addition to the protective coating, the overall architecture of the dosage form, such as for example the structure and arrangement of the SPH body with respect to the active agent delivery regions, the level of compression of the SPH body (if compressed), and composition of components of the dosage form can also be selected to provide a predetermined release of the active agent from the dosage form.

For example, in one embodiment, a release rate for the agent may be at least about 90% within 1 min, as determined by USP Dissolution Assay 711 with Apparatus 1 and a dissolution medium of 150 mM phosphate buffered saline at a pH of 5.0. By way of further example, in one embodiment, a release rate for the agent may be at least about 90% within 1 min, as determined by USP Dissolution Assay 711 with Apparatus 1 and a dissolution medium of 150 mM phosphate buffered saline at a pH of 5.5. By way of further example, in one embodiment, a release rate for the agent may be at least about 90% within 1 min, as determined by USP Dissolution Assay 711 with Apparatus 1 and a dissolution medium of 150 mM phosphate buffered saline at a pH of 6.0. By way of further example, in one embodiment, a release rate for the agent may be at least about 90% within 1 min, as determined by USP Dissolution Assay 711 with Apparatus 1 and a dissolution medium of 150 mM phosphate buffered saline at a pH of 6.5. By way of further example, in one embodiment, a release rate for the agent may be at least about 90% within 1 min, as determined by USP Dissolution Assay 711 with Apparatus 1 and a dissolution medium of 150 mM phosphate buffered saline at a pH of 7.0. By way of further example, in one embodiment, a release rate for the agent may be at least about 90% within 1 min, as determined by USP Dissolution Assay 711 with Apparatus 1 and a dissolution medium of 150 mM phosphate buffered saline at a pH of 7.5.

For example, in one embodiment, a release rate for the agent may be at least about 90% within 10 min, as determined by USP Dissolution Assay 711 with Apparatus 1 and a dissolution medium of 150 mM phosphate buffered saline at a pH of 5.0. By way of further example, in one embodiment, a release rate for the agent may be at least about 90% within 10 min, as determined by USP Dissolution Assay 711 with Apparatus 1 and a dissolution medium of 150 mM phosphate buffered saline at a pH of 5.5. By way of further example, in one embodiment, a release rate for the agent may be at least about 90% within 10 min, as determined by USP Dissolution Assay 711 with Apparatus 1 and a dissolution medium of 150 mM phosphate buffered saline at a pH of 6.0. By way of further example, in one embodiment, a release rate for the agent may be at least about 90% within 10 min, as determined by USP Dissolution Assay 711 with Apparatus 1 and a dissolution medium of 150 mM phosphate buffered saline at a pH of 6.5. By way of further example, in one embodiment, a release rate for the agent may be at least about 90% within 10 min, as determined by USP Dissolution Assay 711 with Apparatus 1 and a dissolution medium of 150 mM phosphate buffered saline at a pH of 7.0. By way of further example, in one embodiment, a release rate for the agent may be at least about 90% within 10 min, as determined by USP Dissolution Assay 711 with Apparatus 1 and a dissolution medium of 150 mM phosphate buffered saline at a pH of 7.5.

For example, in one embodiment, a release rate for the agent may be at least about 90% within 5 min, as determined by USP Dissolution Assay 711 with Apparatus 1 and a dissolution medium of 150 mM phosphate buffered saline at a pH of 5.0. By way of further example, in one embodiment, a release rate for the agent may be at least about 90% within 5 min, as determined by USP Dissolution Assay 711 with Apparatus 1 and a dissolution medium of 150 mM phosphate buffered saline at a pH of 5.5. By way of further example, in one embodiment, a release rate for the agent may be at least about 90% within 5 min, as determined by USP Dissolution Assay 711 with Apparatus 1 and a dissolution medium of 150 mM phosphate buffered saline at a pH of 6.0. By way of further example, in one embodiment, a release rate for the agent may be at least about 90% within 5 min, as determined by USP Dissolution Assay 711 with Apparatus 1 and a dissolution medium of 150 mM phosphate buffered saline at a pH of 6.5. By way of further example, in one embodiment, a release rate for the agent may be at least about 90% within 5 min, as determined by USP Dissolution Assay 711 with Apparatus 1 and a dissolution medium of 150 mM phosphate buffered saline at a pH of 7.0. By way of further example, in one embodiment, a release rate for the agent may be at least about 90% within 5 min, as determined by USP Dissolution Assay 711 with Apparatus 1 and a dissolution medium of 150 mM phosphate buffered saline at a pH of 7.5. In yet another embodiment, a release rate for the agent may be at least about 90% within 30 min, as determined by USP Dissolution Assay 711 with Apparatus 1 and a dissolution medium of 150 mM phosphate buffered saline at a pH of 5.0. By way of further example, in one embodiment, a release rate for the agent may be at least about 90% within 30 min, as determined by USP Dissolution Assay 711 with Apparatus 1 and a dissolution medium of 150 mM phosphate buffered saline at a pH of 5.5. By way of further example, in one embodiment, a release rate for the agent may be at least about 90% within 30 min, as determined by USP Dissolution Assay 711 with Apparatus 1 and a dissolution medium of 150 mM phosphate buffered saline at a pH of 6.0. By way of further example, in one embodiment, a release rate for the agent may be at least about 90% within 30 min, as determined by USP Dissolution Assay 711 with Apparatus 1 and a dissolution medium of 150 mM phosphate buffered saline at a pH of 6.5. By way of further example, in one embodiment, a release rate for the agent may be at least about 90% within 30 min, as determined by USP Dissolution Assay 711 with Apparatus 1 and a dissolution medium of 150 mM phosphate buffered saline at a pH of 7.0. By way of further example, in one embodiment, a release rate for the agent may be at least about 90% within 30 min, as determined by USP Dissolution Assay 711 with Apparatus 1 and a dissolution medium of 150 mM phosphate buffered saline at a pH of 7.5. In yet another embodiment, a release rate for the agent may be at least about 90% within 2 hours, as determined by USP Dissolution Assay 711 with Apparatus 1 and a dissolution medium of 150 mM phosphate buffered saline at a pH of 5.0. By way of further example, in one embodiment, a release rate for the agent may be at least about 90% within 2 hours, as determined by USP Dissolution Assay 711 with Apparatus 1 and a dissolution medium of 150 mM phosphate buffered saline at a pH of 5.5. By way of further example, in one embodiment, a release rate for the agent may be at least about 90% within 2 hours, as determined by USP Dissolution Assay 711 with Apparatus 1 and a dissolution medium of 150 mM phosphate buffered saline at a pH of 6.0. By way of further example, in one embodiment, a release rate for the agent may be at least about 90% within 2 hours, as determined by USP Dissolution Assay 711 with Apparatus 1 and a dissolution medium of 150 mM phosphate buffered saline at a pH of 6.5. By way of further example, in one embodiment, a release rate for the agent may be at least about 90% within 2 hours, as determined by USP Dissolution Assay 711 with Apparatus 1 and a dissolution medium of 150 mM phosphate buffered saline at a pH of 7.0. By way of further example, in one embodiment, a release rate for the agent may be at least about 90% within 2 hours, as determined by USP Dissolution Assay 711 with Apparatus 1 and a dissolution medium of 150 mM phosphate buffered saline at a pH of 7.5.

The oral dosage form may also be configured to provide different layers or structures therein having the active agent, SPH and/or other excipients therein, that provide different rates of release of the active agent and/or SPH from the oral dosage form. For example, in one embodiment the oral dosage form may have a first rate of release of at least one of the active agent and SPH from a first part of the oral dosage form (e.g., a first layer or section of the oral dosage form), and may have a second rate of release of at least one of the active agent and SPH from a second part of the oral dosage form (e.g., a second layer of section of the oral dosage form), that is different from the first rate of release.

According to one embodiment, the oral dosage form is provided in a size that provides good delivery of the active agent in the intestinal tract, without excessively occluding or blocking the intestinal tract. For example, the longest dimension of the oral dosage form may be less than about 3 cm, such as less than about 2 cm, and even less than about 1.5 cm. Typically, the longest dimension of the oral dosage form will be in the range of from about 0.5 cm to about 3 cm, such as from about 1 cm to about 3 cm, and even from about 1 cm to about 2 cm. Suitable capsule sizes may be, for example, size 1, 0, 00 and 000, and including the “EL” versions of any of these sizes.

Method of Treatment

In some embodiments, an oral dosage form may be administered to an individual, patient, or a subject. In some cases, the oral dosage form may be administered as a single dosage. In other embodiments, a plurality of oral dosage forms may be administered to provide multiple dosages over time. Alternatively, the oral dosage form described herein may be administered to a subject in need thereof without food or under a fasting condition. For example, the oral dosage form may be administered at least about 1 hour, at least about 2 hours, at least about 3 hours, at least about 4 hours, at least about 5 hours, at least about 6 hours, at least about 7 hours, at least about 8 hours, at least about 9 hours, at least about 10 hours, at least about 11 hours, at least about 12 hours, between about 3 hours to about 12 hours, between about 4 hours to about 12 hours, between about 4 hours to about 10 hours, between about 4 hours to about 8 hours, or between about 4 hours to about 6 hours, after consumption of food by a subject.

Alternatively, the oral dosage forms described herein may be administered to a subject in need thereof under a condition of fluid restriction. This restriction shall mean that over the stated time, the subject may consume less than 16 oz. of fluids, less than 8 oz of fluids, less than 4 oz of fluids, less than 2 oz of fluids, or less than 1 oz of fluids. For example, the subject may be restricted in their consumption of fluids prior to being administered the oral dosage form for at least about 1 hours, at least about 2 hours, at least about 3 hours, at least about 4 hours, at least about 5 hours, at least about 8 hours, between about 1 hours to about 2 hours, between about 1 hours to about 4 hours. Additionally, the subject may be restricted in their consumption of fluids after being administered the oral dosage form for at least about 1 hours, at least about 2 hours, at least about 3 hours, at least about 4 hours, at least about 5 hours, at least about 8 hours, between about 1 hours to about 2 hours, between about 1 hours to about 4 hours.

Treatment can be continued for as long or as short of a period as desired. The oral dosage form may be administered on a regimen of, for example, one to four or more times per day. A suitable treatment period can be, for example, at least about one week, at least about two weeks, at least about one month, at least about six months, at least about 1 year, or indefinitely. A treatment period can terminate when a desired result is achieved. A treatment regimen can include a corrective phase, during which a dose sufficient, for example, to reduce symptoms is administered, and can be followed by a maintenance phase, during which a lower dose sufficient to maintain the reduced symptoms is administered. A suitable maintenance dose is likely to be found in the lower parts of the dose ranges provided herein, but corrective and maintenance doses can readily be established for individual subjects by those of skill in the art without undue experimentation, based on the disclosure herein.

In certain embodiments, the oral dosage form may be used to deliver an agent (e.g., octreotide) to a subject in need thereof. In some embodiments, the oral dosage form may be capable of delivering insulin to a patient in need thereof, such as a person suffering from diabetes. In certain embodiments, the oral dosage form may be used to deliver an agent (e.g., calcitonin) to a subject in need thereof. For example, the oral dosage form may be used to treat hypercalcemia. In another example, the oral dosage form may be used to treat a bone disease, such as osteoporosis. In yet another embodiment, the oral dosage form may be used to treat a mental disorder, such as bipolar disorder or mania. In yet another embodiment the oral dosage form may deliver an active agent such as a GLP-1 agonist to treat a disorder such as type II diabetes and/or obesity in a patient in need thereof. In yet another embodiment, the oral dosage form may deliver an active agent such as an enzyme-resistant peptide to treat a disorder such as a metabolic disorder to a patient in need thereof.

The oral dosage forms described herein may be used to administer an agent to patients (e.g., animals and/or humans) in need of such treatment in dosages that will provide optimal pharmaceutical efficacy. It will be appreciated that the number and/or type of oral dosage forms required for use in any particular application will vary from patient to patient, not only with the particular agent selected, but also with the concentration of agent in the oral dosage form, the nature of the condition being treated, the age and condition of the patient, concurrent medication or special diets then being followed by the patient, and other factors which those skilled in the art will recognize, with the appropriate dosage ultimately being at the discretion of the attendant physician.

Accordingly, in one embodiment, a method of delivering an active agent to a patient comprises orally administering the oral dosage form described herein, where the oral dosage form has the delivery structure containing the SPH body and active agent delivery regions having active agent at the exterior surface of the SPH body, and protective coating, as described for embodiments of the oral dosage forms above.

### EXAMPLES

### Example 1A

The present example illustrates the preparation of an ion-paired SPH material suitable for use as the SPH body in an oral dosage form. The ion-paired SPH material was prepared using chitosan as the ionically charged structural support polymer, and polymerizing in the presence of a polymerization mixture with monomers comprising acrylic acid, acrylamide, MPEG Acrylate (Mn480), and methylene bisacrylamide as a cross-linking agent.

To prepare the ion-paired SPH material of the current Example, a chitosan solution was formed by combining acrylic acid (49% by weight) with chitosan (˜49% by weight) in deionized water (2% by weight), and allowed to mix for several hours (see Table 1A). The initiator solutions were also prepared shortly before the polymerization reaction, comprising an aqueous ammonium persulfate solution (20% by weight ammonium persulfate), and aqueous tetramethylethylenediamine (TEMED) solution (20% by weight TEMED) (see Tables 1 b and 1c). The chitosan solution was then combined with the deionized water, MPEG acrylate, acrylamide, methylene bisacrylamide, PluronicF127 and NaOH, and allowed to mix completely on a roller mixer, and the pH was checked to verify it was at about 4.9 (see Table 1D for final ingredient amounts). For purposes of evaluating the SPH material, the solution was split into 3 equal aliquots in 15×150 nm test tubes. The ammonium persulfate and TEMED solutions were then sequentially added to the test tubes and briefly stirred, after which 0.5 g of sodium bicarbonate as a foaming agent was added with vigorous mixing for another brief period. Polymerization onset was observed in about 30 seconds following the addition of sodium bicarbonate. The polymerized SPH material was allowed to cure for 30 minutes, following by placing into a 1:2 mixture of deionized water:reagent alcohol for at least one hour, and then in pure reagent alcohol for an additional hour. The SPH material was then manually blotted dry and placed in a drying convection over set to 160° F. for at least 12 hours.

TABLE 1A

| Chitosan Solution | Amount (g) | % |
|---|---|---|
| Acrylic Acid | 4.9 | 49% |
| DI H2O | 4.9 | 49% |
| Chitosan | 0.2 | 2% |

TABLE 1B

| Ammonium Persulfate Solution | Amount (g) | % |
|---|---|---|
| Ammonium Persulfate | 1 | 20% |
| DI H2O | 4 | 80% |

TABLE 1C

| TEMED Solution | Amount (g) | % |
|---|---|---|
| TEMED | 1 | 20% |
| DI H2O | 4 | 80% |

TABLE 1D

| Component | Compound | Weight (g) | Mol Eq. | Wt % (g/g) |
|---|---|---|---|---|
| Monomer | Acrylamide | 1.250 | 0.01759 | 23.25% |
| Monomer | Acrylic Acid | 0.490 | 0.00680 | 9.11% |
| Monomer | MPEG Acrylate | 0.500 | 0.00094 | 9.30% |
| | (Mn 480) | | | |
| Cross-linking | Methylene | 0.008 | 0.00011 | 0.16% |
| Agent | Bisacrylamide | | | |
| Surfactant | PluronicF127 | 0.025 | | 0.47% |
| Ionically | Chitosan | 0.020 | | 0.37% |
| Charged | | | | |
| Structural | | | | |
| Support | | | | |
| Polymer | | | | |
| pH Adjuster | NaOH | 0.333 | | |
| Solvent | Deionized | 2.000 | | |
| | Water | | | |

Total Reaction Mass (g) = 5.376
% Solids = 42.7%

| | | | | |
|---|---|---|---|---|
| Initiator | Ammonium | 0.450 | 0.000394 | 1.67% |
| | Persulfate | | | |
| Initiator | TEMED (20%) | 0.300 | 0.000516 | 1.12% |
| Foaming | Sodium | 0.450 | 0.0053 | 8.37% |
| Agent | Bicarbonate | | | |

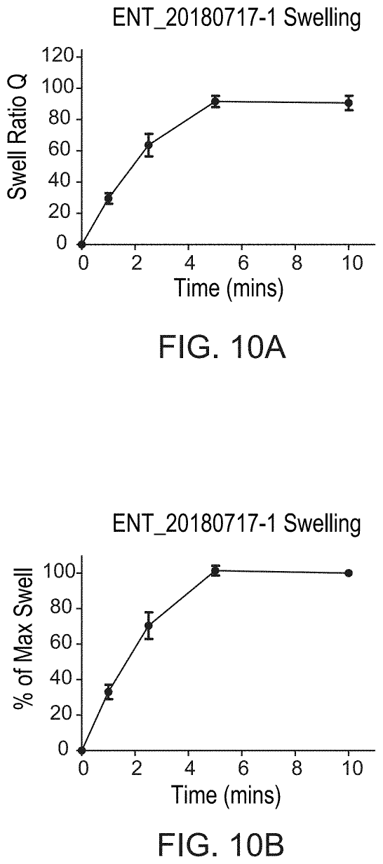

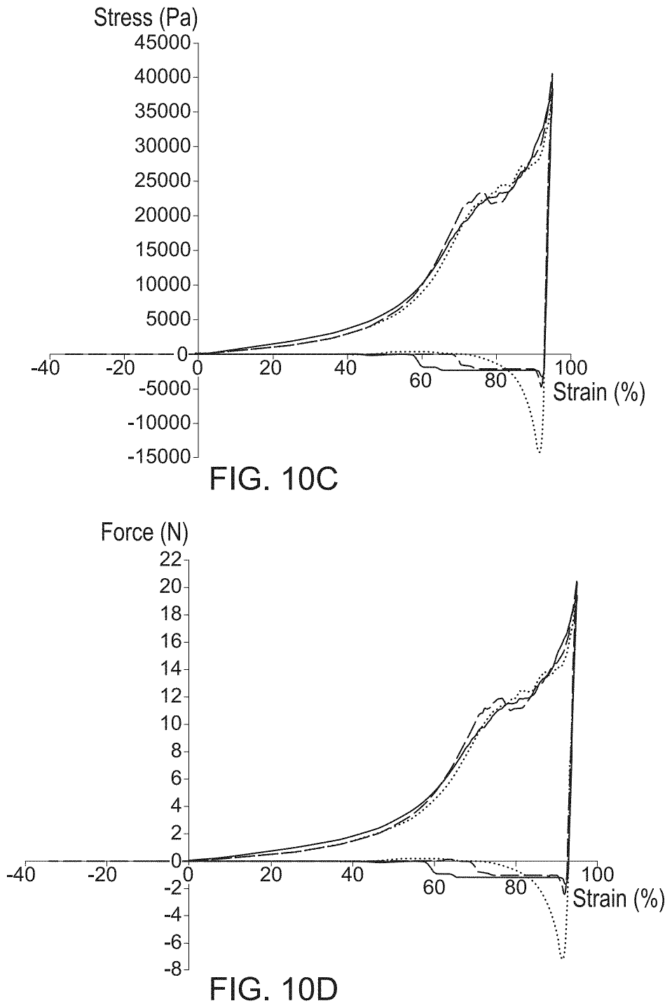

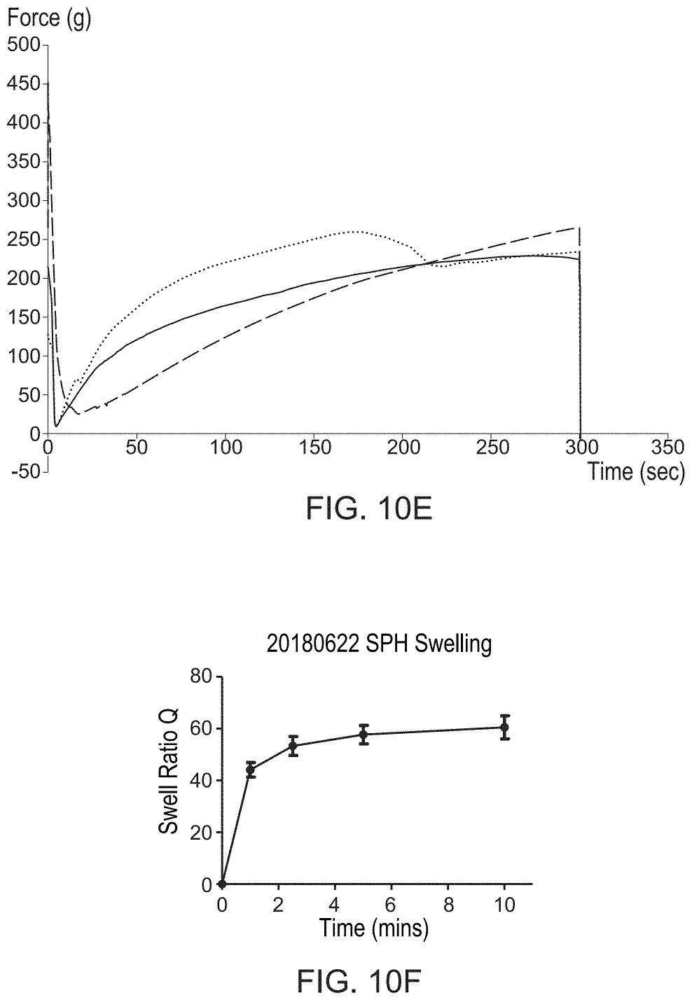

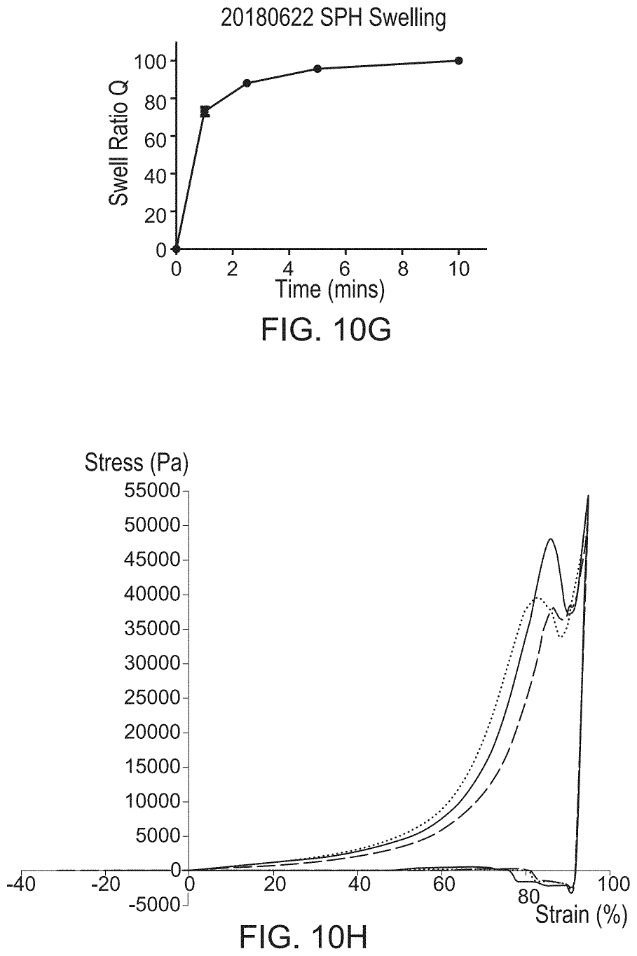

The SPH material prepared according to the above procedure was then characterized to determine the Swelling Ratio and swelling characteristics, as well as strength characteristics such as the Compressive Strength and Radial Force, for each of the three samples prepared. The swelling characteristics and strength characteristics were determined according to procedures as described elsewhere herein. Table 1E below shows results for the Swelling Ratio as determined at 1, 2.5, 5 and 10 minute intervals, along with the Swell Ratio Percentage at each interval, while FIG. 10A is a plot of the Swelling Ratio over time, and FIG. 10B is a plot of the Swell Ratio Percentage over time.

TABLE 1E

| Time (min) | | Description | 0 | 1 | 2.5 | 5 | 10 | |
|---|---|---|---|---|---|---|---|---|
| Mass mg | 1 | ENT_20180717i | 484 | 12828 | 28855 | 45906 | 48010 | |
| | 2 | ENT_20180717ii | 459 | 17113 | 36155 | 44683 | 42424 | |
| | 3 | ENT_20180717iii | 432 | 11992 | 24054 | 36968 | 35985 | |
| Mass Gain | 1 | ENT_20180717i | 0 | 12344 | 28371 | 45422 | 47526 | |
| (mg) | 2 | ENT_20180717ii | 0 | 16654 | 35696 | 44244 | 41965 | |
| | 3 | ENT_20180717iii | 0 | 11560 | 23622 | 36536 | 35553 | |
| | | | | | | | | Average Max |
| Selling | 1 | ENT_20180717i | 0.0 | 25.5 | 58.6 | 93.8 | 98.2 | 93.0 |
| Ratio Q | 2 | ENT_20180717ii | 0.0 | 36.3 | 77.8 | 96.3 | 91.4 | |
| | 3 | ENT_20180717iii | 0.0 | 26.8 | 54.7 | 84.6 | 82.3 | |
| | | | | | | | | Average % @ 1 min |
| % of Max | 1 | ENT_20180717i | 0% | 26% | 60% | 96% | 100% | 33% |
| Swell | 2 | ENT_20180717ii | 0% | 40% | 85% | 105% | 100% | |
| | 3 | ENT_20180717iii | 0% | 33% | 66% | 103% | 100% | |

FIG. 10C is a plot of the stress versus strain measurement with stress in units of Pascals, to characterize the Compressive Strength. FIG. 10D is a plot of the force versus strain measurement with force in units of Newtons, to characterize the Compressive Strength.

Table 1F below summarizes the Compressive Strength results, including the Yield Point, Peak Force Under Compression, and Energy Absorption for each sample.

TABLE 1F

| Start Batch | Approx. | Peak Force | Energy |
|---|---|---|---|
| Hydrogel | Yield | Under | Absorption @ |
| Compression | Point | Compression | 95% Strain |
| Strain TA-25 | (Pa) | (g) | (J/m\hat{ }3) |
| ENT_20180717_strain1 | 25,000 | 1,984 | 981,858 |
| ENT_20180717_strain2 | 25,000 | 1,956 | 932,847 |
| ENT_20180717_strain3 | 25,000 | 2,109 | 968,002 |
| Average: | 25,000 | 2,016 | 960,902 |
| S.D. | — | 81 | 25,265 |

FIG. 10E is a plot of the force exerted over time in characterizing the Radial Force of the samples. Table 1G below summarizes the Radial Force characterizations, including the Peak Swell Force and Impulse for each sample.

TABLE 1G

| Start Batch | | |
|---|---|---|
| Hydrogel Fixed | Impulse @ | Peak Swell |
| Distance Swell - | 5 mins (g*s) | Force (g) |
| ENT_20180717_swell1 | 52,665 | 230 |
| ENT_20180717_swell2 | 61,710 | 261 |
| ENT_20180717_swell3 | 47,478 | 266 |
| Average: | 53,951 | 252 |
| S.D. | 7,202 | 20 |

The samples as tested having the ion-paired structure using chitosan as a structural support polymer exhibited excellent swelling characteristics, including Swelling Ratio, Swelling Speed, and Swell Ratio Percentage, while also exhibiting excellent strength properties such as Radial Force and Compressive Strength properties.

### Example 1B

The present example illustrates the preparation of a yet another ion-paired SPH material suitable for use as the SPH body in an oral dosage form. As with Example 1A, the ion-paired SPH material was prepared using chitosan as the ionically charged structural support polymer, and polymerizing in the presence of a polymerization mixture with monomers comprising acrylic acid, acrylamide, MPEG Acrylate (Mn480), and methylene bisacrylamide as a cross-linking agent.

The ion-paired SPH material as prepared according to the method described in Example 1A, with the final ingredient amounts/ratios as set forth in Table 1H below.

TABLE 1H

| Component | Compound | Weight (g) | Mol Eq. | Wt % (g/g) |
|---|---|---|---|---|
| Monomer | Acrylamide | 1.250 | 0.01759 | 28.45% |
| Monomer | Acrylic Acid | 0.490 | 0.00680 | 11.15% |
| Monomer | MPEG Acrylate | 0.500 | 0.00094 | 11.38% |
| | (Mn 480) | | | |
| Cross-linking | Methylene | 0.025 | 0.00032 | 0.57% |
| Agent | Bisacrylamide | | | |
| Surfactant | PluronicF127 | 0.025 | | 0.57% |
| Ionically | Chitosan | 0.020 | | 0.46% |
| Charged | | | | |
| Structural | | | | |
| Support | | | | |
| Polymer | | | | |
| pH Adjuster | 5M NaOH | 0.333 | | |
| Solvent | Deionized | 1.000 | | |
| | Water | | | |

Total Reaction Mass (g) = 4.393
% Solids = 52.6%

| | | | | |
|---|---|---|---|---|
| Initiator | Ammonium | 0.450 | 0.000394 | 2.05% |
| | Persulfate | | | |
| Initiator | TEMED (20%) | 0.300 | 0.000516 | 1.37% |
| Foaming | Sodium | 0.500 | 0.0059 | 11.38% |
| Agent | Bicarbonate | | | |

The SPH material prepared according to the above procedure was then characterized to determine the Swelling Ratio and swelling characteristics, as well as strength characteristics such as the Compressive Strength and Radial Force, for each of the three samples prepared. The swelling characteristics and strength characteristics were determined according to procedures as described elsewhere herein. Table 11 below shows results for the Swelling Ratio as determined at 1, 2.5, 5 and 10 minute intervals, along with the Swell Ratio Percentage at each interval, while FIG. 10F is a plot of the Swelling Ratio over time, and FIG. 10G is a plot of the Swell Ratio Percentage over time.

TABLE 1I

| Time (min) | | Description | 0 | 1 | 2.5 | 5 | 10 | |
|---|---|---|---|---|---|---|---|---|
| Mass (mg) | 1 | ENT_20180622i | 434 | 21954 | 26351 | 28059 | 29978 | |
| | 2 | ENT_20180622ii | 453 | 18877 | 21742 | 23698 | 24265 | |
| | 3 | ENT_20180622iii | 375 | 16139 | 20310 | 22132 | 23175 | |
| Mass Gain | 1 | ENT_20180622i | 0 | 21520 | 25917 | 27625 | 29544 | |
| (mg) | 2 | ENT_20180622ii | 0 | 18424 | 21289 | 23245 | 23812 | |
| | 3 | ENT_20180622iii | 0 | 15764 | 19935 | 21757 | 22800 | |
| | | | | | | | | Average |
| Swelling | 1 | ENT_20180622i | 0.0 | 49.6 | 59.7 | 63.7 | 68.1 | 60.5 |
| Ratio Q | 2 | ENT_20180622ii | 0.0 | 40.7 | 47.0 | 51.3 | 52.6 | |
| | 3 | ENT_20180622iii | 0.0 | 42.0 | 53.2 | 58.0 | 60.8 | |
| | | | | | | | | Average |
| % of Max | 1 | ENT_20180622i | 0% | 73% | 88% | 94% | 100% | 73% |
| Swell | 2 | ENT_20180622ii | 0% | 77% | 89% | 98% | 100% | |
| | 3 | ENT_20180622iii | 0% | 69% | 87% | 95% | 100% | |

FIG. 10H is a plot of the stress versus strain measurement with stress in units of Pascals, to characterize the Compressive Strength.

Table 1J below summarizes the Compressive Strength results, including the Yield Point, Peak Force Under Compression, and Energy Absorption for each sample.

TABLE 1J

| Start Batch | Approx. | Peak Force | Energy |
|---|---|---|---|
| Hydrogel | Yield | Under | Absorption @ |
| Compression | Point | Compression | 95% Strain |
| Strain TA-25 | (Pa) | (g) | (J/m\hat{ }3) |
| 20180622_strain1 | 40,000 | 2,819 | 1,168,543 |
| 20180622_strain2 | 50,000 | 2,777 | 1,216,069 |
| 20180622_strain3 | 40,000 | 2,614 | 950,125 |
| Average: | 43,333 | 2,737 | 1,111,579 |

FIG. 10I is a plot of the force exerted over time in characterizing the Radial Force of the samples.

Table 1K below summarizes the Radial Force characterizations, including the Peak Swell Force and Impulse for each sample.

TABLE 1K

| Start Batch | | |
|---|---|---|
| Hydrogel Fixed | Impulse, | Peak |
| Distance Swell - | 5 mins (g*s) | Force (g) |
| 20180622-1-swell1 | 23,657 | 92 |
| 20180622-1-swell2 | 16,773 | 62 |
| 20180622-1-swell3 | 25,211 | 97 |
| Average: | 21,881 | 84 |

The samples as tested having the ion-paired structure using chitosan as a structural support polymer exhibited excellent swelling characteristics, including Swelling Ratio, Swelling Speed, and Swell Ratio Percentage, while also exhibiting excellent strength properties such as Radial Force and Compressive Strength properties.

### Example 2

The present example illustrates the preparation of a cationic SPH material prepared using cationic monomers, suitable for use as the SPH body in an oral dosage form. The SPH material was prepared using (3-acrylaminopropyl) trimethyl ammonium chloride as the cationically charged monomer, and polymerizing in a polymerization mixture with monomers comprising acrylamide and MPEG Acrylate (Mn480), with methylene bisacrylamide as a cross-linking agent.

To prepare the cationic SPH material of the current Example, initiator solutions were prepared shortly before the polymerization reaction, comprising an aqueous ammonium persulfate solution (20% by weight ammonium persulfate), and aqueous tetramethylethylenediamine (TEMED) solution (20% by weight TEMED) (see Tables 2A and 2B). The polymerization mixture was formed by combining the (3-acrylaminopropyl) trimethyl ammonium chloride with deionized water, MPEG acrylate, acrylamide, methylene bisacrylamide, PluronicF127, acetic acid and NaOH, and allowed to mix completely on a roller mixer, and the pH was checked to verify it was at about 4.75-5 (see Table 2C for final ingredient amounts). For purposes of evaluating the SPH material, the solution was split into 3 equal aliquots in 15×150 nm test tubes. The ammonium persulfate and TEMED solutions were then sequentially added to the test tubes and briefly stirred, after which 0.5 g of sodium bicarbonate as a foaming agent was added with vigorous mixing for another brief period. Polymerization onset was observed in about 30 seconds following the addition of sodium bicarbonate. The polymerized SPH material was allowed to cure for 30 minutes, following by placing into a 1:2 mixture of deionized water:reagent alcohol for at least one hour, and then in pure reagent alcohol for an additional hour. The SPH material was then manually blotted dry and placed in a drying convection over set to 160° F. for at least 12 hours.

TABLE 2A

| Ammonium Persulfate Solution | Amount (g) | % |
|---|---|---|
| Ammonium Persulfate | 1 | 20% |
| DI H2O | 4 | 80% |

TABLE 2B

| TEMED Solution | Amount (g) | % |
|---|---|---|
| TEMED | 1 | 20% |
| DI H2O | 4 | 80% |

TABLE 2C

| | | Weight | Mol | Wt % |
|---|---|---|---|---|
| Component | Compound | (g) | Eq. | (g/g) |
| Monomer | Acrylamide | 1.250 | 0.01759 | 26.49% |
| Monomer | (3-Acrylamidopropyl) | 1.000 | 0.00363 | 15.90% |
| | TMA Cl | | | |
| Monomer | MPEG Acrylate (Mn | 0.500 | 0.00032 | 0.53% |
| | 480) | | | |
| Cross-linking | Methylene | 0.025 | 0.00011 | 0.16% |
| Agent | Bisacrylamide | | | |
| Surfactant | PluronicF127 | 0.025 | | 0.53% |
| Foaming | Acetic Acid | 0.085 | | 1.80% |
| Agent | | | | |
| Solvent | Deionized Water | 2.000 | | |

Total Reaction Mass (g) = 4.718
% Solids = 54.0%

| | | | | |
|---|---|---|---|---|
| Initiator | Ammonium | 0.300 | 0.000263 | 1.27% |
| | Persulfate | | | |
| Initiator | TEMED (20%) | 0.200 | 0.000344 | 0.85% |
| Foaming | Sodium Bicarbonate | 0.250 | 0.0030 | 5.30% |
| Agent | | | | |
| pH adjusting | 5M NaOH | 0.067 | | |
| Agent | | | | |

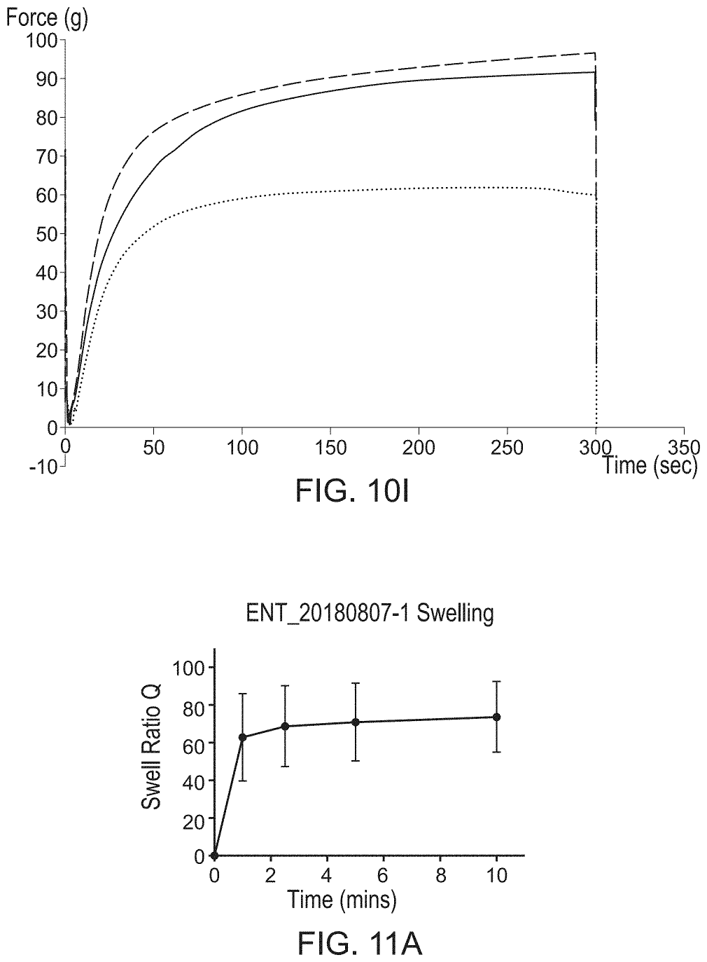

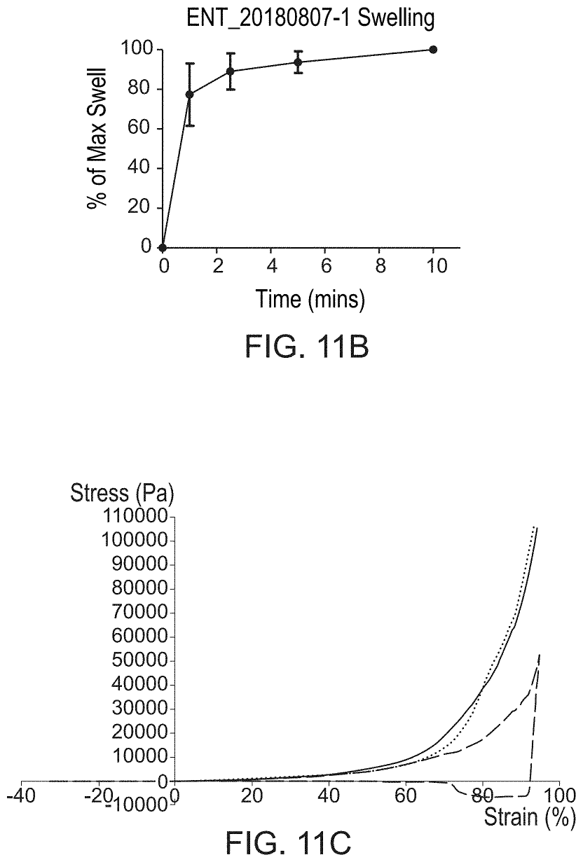

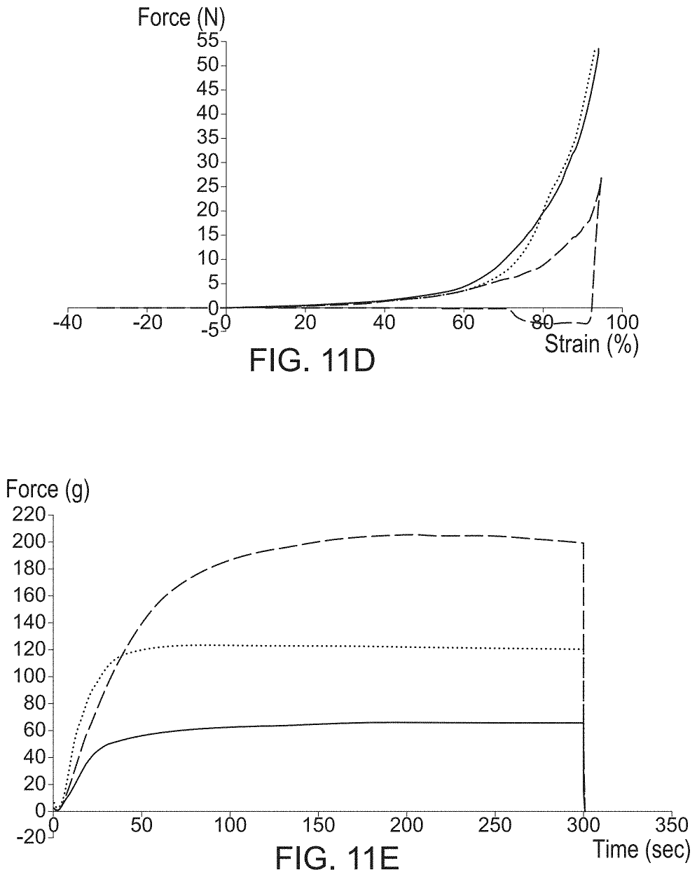

The SPH material prepared according to the above procedure was then characterized to determine the Swelling Ratio and other swelling characteristics, as well as strength characteristics such as the Compressive Strength and Radial Force, for each of the three samples prepared. The swelling characteristics and strength characteristics were determined according to procedures as described elsewhere herein. Table 2D below shows results for the Swelling Ratio as determined at 1, 2.5, 5 and 10 minute intervals, along with the Swell Ratio Percentage at each interval, while FIG. 11A is a plot of the Swelling Ratio over time, and FIG. 11B is a plot of the Swell Ratio Percentage over time.

TABLE 2D

| Time (min) | | Description | 0 | 1 | 2.5 | 5 | 10 | |
|---|---|---|---|---|---|---|---|---|
| Mass (mg) | 1 | 20180807_1 i | 398 | 33066 | 34215 | 35103 | 36088 | |
| | 2 | 20180807_1 ii | 378 | 34085 | 36458 | 36480 | 36259 | |
| | 3 | 20180807_1 iii | 543 | 9555 | 14509 | 16890 | 20226 | |
| Mass Gain | 1 | 20180807_1 i | 0 | 32668 | 33817 | 34705 | 35690 | |
| (mg) | 2 | 20180807_1 ii | 0 | 33707 | 36080 | 36102 | 35881 | |
| | 3 | 20180807_1 iii | 0 | 9012 | 13966 | 16347 | 19683 | |
| | | | | | | | | Average |
| Swelling | 1 | 20180807_1 i | 0.0 | 82.1 | 85.0 | 87.2 | 89.7 | 73.6 |
| Ratio Q | 2 | 20180807_1 ii | 0.0 | 89.2 | 95.4 | 95.5 | 94.9 | |
| | 3 | 20180807_1 iii | 0.0 | 16.6 | 25.7 | 30.1 | 36.2 | |
| | | | | | | | | Average |
| % of Max | 1 | 20180807_1 i | 0% | 92% | 95% | 97% | 100% | 77% |
| | 2 | 20180807_1 ii | 0% | 94% | 101% | 101% | 100% | |
| | 3 | 20180807_1 iii | 0% | 46% | 71% | 83% | 100% | |

FIG. 110 is a plot of the stress versus strain measurement with stress in units of Pascals, to characterize the Compressive Strength. FIG. 11D is a plot of the force versus strain measurement with force measured in Newtons, to characterize the Compressive Strength.

Table 2E below summarizes the Compressive Strength results, including the Yield Point, Peak Force Under Compression, and Energy Absorption for each sample.

TABLE 2E

| | Approx. | Peak | Energy |
|---|---|---|---|
| | Yield | Force Under | Absorption @ |
| Start Batch Hydrogel | Point | Compression | 95% Strain |
| Compression Strain TA-25 | (Pa) | (g) | (J/m\hat{ }3) |
| 20180807_cationic_strain1 | 85,000 | 5,482 | 1,476,146 |
| 20180807_cationic_strain2 | 85,000 | 5,500 | 1,353,681 |
| 20180807_cationic_strain3 | 40,000 | 2,760 | 840,888 |
| Average: | 70,000 | 4,581 | 1,223,572 |
| S.D. | 25,981 | 1,577 | 337,023 |

FIG. 11E is a plot of the force exerted over time in characterizing the Radial Force of the samples.

Table 2F below summarizes the Radial Force characterizations, including the Peak Swell Force and Impulse for each sample.

TABLE 2F

| Start Batch Hydrogel | Impulse @ | Peak |
|---|---|---|
| Fixed Distance Swell - | 5 min (g*s) | Force (g) |
| 20180807_cationic_swell1 | 17,992 | 67 |
| 20180807_cationic_swell1 | 34,771 | 124 |
| 20180807_cationic_swell1 | 52,524 | 206 |
| Average: | 35,096 | 133 |
| S.D. | 17,269 | 70 |

The samples as tested having the cationic SPH formed from monomer containing cationically charged groups exhibited excellent swelling characteristics, including Swelling Ratio, Swelling Speed, and Swell Ratio Percentage, while also exhibiting excellent strength properties such as Radial Force and Compressive Strength properties.

### Example 3

In this example, the ion-paired SPH material comprising the ionically charged structural support polymer of Example 1B (Ion-Paired SPH A), was compared to compared to an SPH material having the same composition, but without any ionically charged structural support polymer incorporated therein (Comparative SPH B). The Comparative SPH B material was prepared according to a method such as that described in Examples 1A-1B above, with the exception that chitosan was not added for the Comparative SPH material.

Table 3a below provides the ingredient amounts/ratios for the polymerization mixture use to form the Comparative SPH B.

TABLE 3a
Comparative SPH B

| Component | Compound | Weight (g) | Mol Eq. | Wt % (g/g) |
|---|---|---|---|---|
| Monomer | Acrylamide | 1.250 | 0.01759 | 28.52% |
| Monomer | Acrylic Acid | 0.500 | 0.00680 | 11.41% |
| Monomer | MPEG Acrylate | 0.500 | 0.00094 | 11.41% |
| | (Mn 480) | | | |
| Cross-linking | Methylene | 0.025 | 0.00032 | 0.57% |
| Agent | Bisacrylamide | | | |
| Surfactant | PluronicF127 | 0.025 | | 0.57% |
| Ionically | None | | | |
| Charged | | | | |
| Structural | | | | |
| Support | | | | |
| Polymer | | | | |
| pH Adjuster | 5M NaOH | 0.333 | | |
| Solvent | Deionized | 1.000 | | |
| | Water | | | |

Total Reaction Mass (g) = 4.383
% Solids = 52.5%

| | | | | |
|---|---|---|---|---|
| Initiator | Ammonium | 0.450 | 0.000394 | 2.05% |
| | Persulfate | | | |
| Initiator | TEMED (20%) | 0.300 | 0.000516 | 1.37% |
| Foaming | Sodium | 0.450 | 0.0053 | 10.27% |
| Agent | Bicarbonate | | | |

Referring to FIG. 10H for the Ion-Paired SPHA prepared in Example 1B, it can be seen that the material exhibits excellent compressive strength values, in terms of the Yield Point, Peak Force Under Compression, and Energy Absorption. Specifically, referring to Table 1J in Example 1B, the Ion-Paired SPH A exhibited an excellent Yield Point of 43,333 Pa on average, with an average Peak Force Under Compression of 2,737 g and an average Energy Absorption of 1,111,579 J/m³.

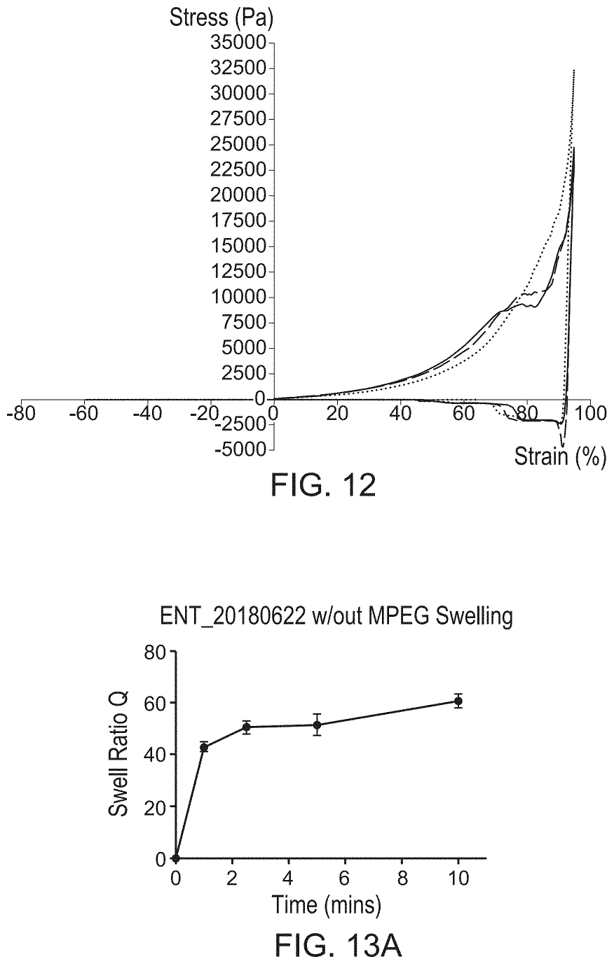

By comparison, FIG. 12 demonstrates the Compressive Strength as evidenced by the Yield Point, of the Comparative SPH B without any ionically charged structural support polymer.

Table 3B summarizes the Compressive Strength results for the Comparative SPH B, in terms of the Yield Point, Peak Force Under Compression, and Energy Absorption.

TABLE 3B

| | | Peak force | Energy |
|---|---|---|---|
| | Approximate | Under | Absorption @ |
| | Yield | Compression | 95% Strain |
| | Point (Pa) | (g) | (J/M\hat{ }3) |
| ENT_20180628- | 12,500.0 | 1,180.2 | 467,503.0 |
| 3_Strain1 | | | |
| ENT_20180628- | 11,000.0 | 1,685.1 | 503,817.2 |
| 3_Strain2 | | | |
| ENT_20180628- | 10,000.0 | 1,287.0 | 465,11.4 |
| 3_Strain3 | | | |
| Average | 11,166.7 | 1,384.1 | 478,813.9 |

Notably, the Comparative SPH B exhibited a dramatically reduced Yield Point as compared to the Ion-Paired SPH A, of only about 11,166.7 Pa on average, or almost ¼ the Compressive Strength of the Ion-Paired SPH A in terms of the Yield Point. Similarly, the Comparative SPH B exhibited a reduced average Peak Force Under Compression of 1,384.1 g and a reduced average Energy Absorption of 478,813.9 J/m³, about half the values of the Ion-Paired SPH A. Accordingly, the results demonstrate that the presence of the ionically charged structural support polymer can drastically improve the strength characteristics of the SPH material, which characteristics may render the SPH material suitable for use in environments such as the gastrointestinal environment where high compressive forces may exist.

### Example 4

In this example, comparative SPH samples were prepared to test the effect of chitosan and MPEG acrylate on the properties of the resulting composition. In this example, an SPH sample formed from a polymerization mixture comprising both chitosan and MPEG acrylate was prepared (Base Formulation), along with an SPH sample formed from a polymerization mixture without MPEG Acrylate (No MPEG Acrylate Formulation), a SPH sample with relatively high levels of chitosan (High Chitosan Formulation) and a SPH sample with no chitosan added (No Chitosan Formulation. The comparative results are described below.

The formulations were each prepared according to a method as described in Examples 1A and 1B.

The Baseline Formulation used final ingredient ratios/amounts as described for Example 1B above, and as provided in Table 1J.

The No MPEG Acrylate Formulation final ingredient ratios/amounts are in Table 4A below.

TABLE 4A
No MPEG Acrylate Formulation

| | | Weight | | Wt % |
|---|---|---|---|---|
| Component | Compound | (g) | Mol Eq. | (g/g) |
| Monomer | Acrylamide | 1.250 | 0.01759 | 32.27% |
| Monomer | Acrylic Acid | 0.490 | 0.00680 | 12.65% |
| Monomer | MPEG Acrylate | None | | |
| | (Mn 480) | | | |
| Cross-linking | Methylene | 0.025 | 0.00032 | 0.65% |
| Agent | Bisacrylamide | | | |
| Surfactant | PluronicF127 | 0.025 | | 0.65% |
| Ionically Charged | Chitosan | 0.020 | | 0.46% |
| Structural Support | | | | |
| Polymer | | | | |
| pH Adjuster | 5M NaOH | 0.333 | | |
| Solvent | Deionized | 1.000 | | |
| | Water | | | |

Total Reaction Mass (g) = 3.873
% Solids = 46.2%

| | | | | |
|---|---|---|---|---|
| Initiator | Ammonium | 0.450 | 0.000394 | 2.32% |
| | Persulfate | | | |
| Initiator | TEMED (20%) | 0.300 | 0.000516 | 1.55% |
| Foaming Agent | Sodium | 0.450 | 0.0059 | 11.62% |
| | Bicarbonate | | | |

The High Chitosan Formulation final ingredient ratios/amounts are in Table 4B below.

TABLE 4B
High Chitosan Formulation

| | | Weight | | Wt % |
|---|---|---|---|---|
| Component | Compound | (g) | Mol Eq. | (g/g) |
| Monomer | Acrylamide | 1.250 | 0.01759 | 32.27% |
| Monomer | Acrylic Acid | 0.490 | 0.00680 | 12.65% |
| Monomer | MPEG Acrylate | 0.500 | 0.00094 | 11.18% |
| | (Mn 480) | | | |
| Cross-linking | Methylene | 0.025 | 0.00032 | 0.65% |
| Agent | Bisacrylamide | | | |
| Surfactant | PluronicF127 | 0.025 | | 0.65% |
| Ionically Charged | Chitosan | 0.100 | | 2.24% |
| Structural Support | | | | |
| Polymer | | | | |
| pH Adjuster | 5M NaOH | 0.333 | | |
| Solvent | Deionized | 1.000 | | |
| | Water | | | |

Total Reaction Mass (g) = 4.473
% Solids = 53.4%

| | | | | |
|---|---|---|---|---|
| Initiator | Ammonium | 0.450 | 0.000394 | 2.01% |
| | Persulfate | | | |
| Initiator | TEMED (20%) | 0.300 | 0.000516 | 1.34% |
| Foaming Agent | Sodium | 0.450 | 0.0059 | 10.06% |
| | Bicarbonate | | | |

The No Chitosan Formulation final ingredient ratios/amounts are in Table 4C below.

TABLE 4C
No Chitosan Formulation

| | | Weight | | Wt % |
|---|---|---|---|---|
| Component | Compound | (g) | Mol Eq. | (g/g) |
| Monomer | Acrylamide | 1.250 | 0.01759 | 28.58% |
| Monomer | Acrylic Acid | 0.490 | 0.00680 | 11.21% |
| Monomer | MPEG Acrylate | 0.500 | 0.00094 | 11.43% |
| | (Mn 480) | | | |
| Cross-linking | Methylene | 0.025 | 0.00032 | 0.57% |
| Agent | Bisacrylamide | | | |
| Surfactant | PluronicF127 | 0.025 | | 0.57% |
| Ionically Charged | None | | | |
| Structural Support | | | | |
| Polymer | | | | |
| pH Adjuster | 5M NaOH | 0.333 | | |
| Solvent | Deionized | 1.000 | | |
| | Water | | | |

Total Reaction Mass (g) = 4.373
% Solids = 52.4%

| | | | | |
|---|---|---|---|---|
| Initiator | Ammonium | 0.450 | 0.000394 | 2.06% |
| | Persulfate | | | |
| Initiator | TEMED (20%) | 0.300 | 0.000516 | 1.37% |
| Foaming Agent | Sodium | 0.450 | 0.0059 | 10.29% |
| | Bicarbonate | | | |

The SPH material (Baseline, No MPEG Acrylate, High Chitosan and No Chitosan) as prepared were then characterized to determine swelling characteristics such as the Swelling Ratio, as well as strength characteristics such as the Compressive Strength and Radial Force, for each of the three samples prepared. The swelling characteristics and strength characteristics were determined according to procedures as described elsewhere herein.

For the Baseline Formulation, the swelling characteristics and strength characteristics are as set forth in Example 1B above.

For the No MPEG Acrylate Formulation, Table 4D below shows results for the Swelling Ratio as determined at 1, 2.5, 5 and 10 minute intervals, along with the Swell Ratio Percentage at each interval, while FIG. 13A is a plot of the Swelling Ratio over time.

TABLE 4D
No MPEG Acrylate Formulation

| Time (min) | | Description | 0 | 1 | 2.5 | 5 | 10 | |
|---|---|---|---|---|---|---|---|---|
| Mass (mg) | 1 | No MPEG i | 368 | 17647 | 20801 | 21833 | 24654 | |
| | 2 | No MPEG ii | 534 | 22961 | 27357 | 28488 | 31727 | |
| | 3 | No MPEG iii | 375 | 16139 | 20310 | 22132 | 23175 | |
| Mass Gain | 1 | No MPEG i | 0 | 17279 | 20433 | 21465 | 24286 | |
| (mg) | 2 | No MPEG ii | 0 | 22427 | 26823 | 27954 | 31193 | |
| | 3 | No MPEG iii | 0 | 19183 | 21833 | 20814 | 27115 | |
| | | | | | | | | Average |
| Swelling | 1 | No MPEG i | 0.0 | 47.0 | 55.5 | 58.3 | 66.0 | 60.7 |
| Ratio Q | 2 | No MPEG ii | 0.0 | 42.0 | 50.2 | 52.3 | 58.4 | |
| | 3 | No MPEG iii | 0.0 | 40.7 | 46.4 | 44.2 | 57.6 | |
| | | | | | | | | Average |
| % of Max | 1 | No MPEG i | 0% | 71% | 84% | 88% | 100% | 71% |
| Swell | 2 | No MPEG ii | 0% | 72% | 86% | 90% | 100% | |
| | 3 | No MPEG iii | 0% | 71% | 81% | 77% | 100% | |

For the High Chitosan Formulation, Table 4E below shows results for the Swelling Ratio as determined at 1, 2.5, 5 and 10 minute intervals, along with the Swell Ratio Percentage at each interval, while FIG. 13B is a plot of the Swelling Ratio over time.

TABLE 4E

| Time (min) | | Description | 0 | 1 | 2.5 | 5 | 10 | |
|---|---|---|---|---|---|---|---|---|
| Mass (mg) | 1 | High Chitosan i | 995 | 7262 | 9551 | 11401 | 13754 | |
| | 2 | High Chitosan ii | 727 | 11590 | 13804 | 16595 | 19537 | |
| | 3 | High Chitosan iii | 787 | 9596 | 13157 | 15885 | 18340 | |
| Mass Gain | 1 | High Chitosan i | 0 | 6267 | 8556 | 10406 | 12759 | |
| (mg) | 2 | High Chitosan ii | 0 | 10863 | 13077 | 15868 | 18810 | |
| | 3 | High Chitosan iii | 0 | 8809 | 12370 | 15098 | 17553 | |
| | | | | | | | | Average |
| Swelling | 1 | High Chitosan i | 0.0 | 6.3 | 8.6 | 10.5 | 12.8 | |
| Ratio Q | 2 | High Chitosan ii | 0.0 | 14.9 | 18.0 | 21.8 | 25.9 | 20.3 |
| | 3 | High Chitosan iii | 0.0 | 11.2 | 15.7 | 19.2 | 22.3 | |
| | | | | | | | | Average |
| % of Max | | High Chitosan i | 0% | 49% | 67% | 82% | 100% | |
| Swell | 2 | High Chitosan ii | 0% | 58% | 70% | 84% | 100% | 52% |
| | 3 | High Chitosan iii | 0% | 50% | 70% | 86% | 100% | |

For the No Chitosan Formulation, Table 4F below shows results for the Swelling Ratio as determined at 1, 2.5, 5 and 10 minute intervals, along with the Swell Ratio Percentage at each interval, while FIG. 13C is a plot of the Swelling Ratio over time.

TABLE 4F

| Time (min) | | Description | 0 | 1 | 2.5 | 5 | 10 | |
|---|---|---|---|---|---|---|---|---|
| Mass (mg) | 1 | No Chitosan i | 538 | 30974 | 42018 | 47021 | 44881 | |
| | 2 | No Chitosan ii | 450 | 12023 | 38652 | 39684 | 41361 | |
| | 3 | No Chitosan iii | 425 | 14856 | 18495 | 30359 | 36413 | |
| Mass Gain | 1 | No Chitosan i | 0 | 30436 | 41480 | 46483 | 44343 | |
| (mg) | 2 | No Chitosan ii | 0 | 11573 | 38202 | 39234 | 40911 | |
| | 3 | No Chitosan iii | 0 | 14431 | 18070 | 29934 | 35988 | |
| | | | | | | | | Average |
| Swelling | 1 | No Chitosan i | 0.0 | 56.6 | 77.1 | 86.4 | 82.4 | 86.0 |
| Ratio Q | 2 | No Chitosan ii | 0.0 | 25.7 | 84.9 | 87.2 | 90.9 | |
| | 3 | No Chitosan iii | 0.0 | 34.0 | 42.5 | 70.4 | 84.7 | |
| | | | | | | | | Average |
| % of Max | 1 | No Chitosan i | 0% | 69% | 94% | 105% | 100% | 46% |
| Swell | 2 | No Chitosan ii | 0% | 28% | 93% | 96% | 100% | |
| | 3 | No Chitosan iii | 0% | 40% | 50% | 83% | 100% | |

The comparative results for the swelling characteristics of each formulation is shown in Table 4G below, and FIGS. 13D-13E.

TABLE 4G
Hydrogel Swell Kinetics

| | Swell | |
|---|---|---|
| Formulation | Ratio Q | Swell % at 1 Minute |
| ENT_20180622 | 60.5 | 73 |
| ENT_20180622 No MPEG | 60.7 | 71 |
| ENT_20180622 High Chitosan | 20.1 | 52 |
| ENT_20180622 No Chitosan | 86 | 46 |

The strength characteristics of each of the formulations was also assessed. As discussed above, the strength characteristics of the Baseline Formulation as set out in Example 1B above.

With respect to the No MPEG Acrylate Formulation, FIG. 13F is a plot of the stress versus strain measurement with stress in units of Pascals, to characterize the Compressive Strength, and Table 4H sets out Compressive Strength measurements including the Yield Point, Peak Force Under Compression, and Energy Absorption.

TABLE 4H

| Start Batch | | Peak Force | Energy |
|---|---|---|---|
| Hydrogel | Approximate | Under | Absorption |
| Compression | Yield | Compression | @ 95% |
| Strain TA-25- | Point (Pa) | (g) | Strain (J/m\hat{ }3) |
| no-mpeg-strain-1 | 18,000 | 1,530 | 810,064 |
| no-mpeg-strain-2 | 20,000 | 2,197 | 814,222 |
| no-mpeg-strain-3 | 32,000 | 2,191 | 982,354 |
| Average: | 23,333 | 1,973 | 868,880 |
| S.D. | 7,572 | 384 | 98,293 |

With respect to the High Chitosan Formulation, FIG. 13G is a plot of the stress versus strain measurement with stress in units of Pascals, to characterize the Compressive Strength, and Table 41 sets out Compressive Strength measurements including the Yield Point, Peak Force Under Compression, and Energy Absorption.

TABLE 4I

| Start Batch | | Peak Force | Energy |
|---|---|---|---|
| Hydrogel | Approximate | Under | Absorption |
| Compression | Yield | Compression | @ 95% |
| Strain TA-25- | Point (Pa) | (g) | Strain (J/m\hat{ }3) |
| hi-chitosan-strain-1 | 65,000 | 5,415 | 2,377,970 |
| hi-chitosan-strain-2 | 35,000 | 2,964 | 1,294,791 |
| hi-chitosan-strain-3 | 22,000 | 2,127 | 728,103 |
| Average: | 40,667 | 3,502 | 1,466,954 |
| S.D. | 22,053 | 1,709 | 838,299 |

With respect to the No Chitosan Formulation, FIG. 13H is a plot of the stress versus strain measurement with stress in units of Pascals, to characterize the Compressive Strength, and Table 4J sets out Compressive Strength measurements including the Yield Point, Peak Force Under Compression, and Energy Absorption.

TABLE 4J

| Start Batch | | Peak Force | Energy |
|---|---|---|---|
| Hydrogel | Approximate | Under | Absorption |
| Compression | Yield | Compression | @ 95% |
| Strain TA-25- | Point (Pa) | (g) | Strain (J/m\hat{ }3) |
| no-chitosan-strain-1 | 15,000 | 1,153 | 586,733 |
| no-chitosan-strain-2 | 15,000 | 1,347 | 599,974 |
| no-chitosan-strain-3 | 18,000 | 1,421 | 561,395 |
| Average: | 16,000 | 1,307 | 582,700 |
| S.D. | 1,732 | 138 | 19,603 |

Table 4K below, along with FIGS. 131-13K, summarize the Compressive Strength results for the formulations.

TABLE 4K
Compression Strength (N = 3)

| | Approximate | | Peak Force Under | | Energy Absorption @ | |
|---|---|---|---|---|---|---|
| Formulation | Yield Point (Pa) | Stdev | Compression (g) | Stdev | 95% Strain (J/m^{∧}3) | Stdev |
| ENT_20180622 | 43,333 | 5,774 | 2,737 | 108 | 1,111,580 | 141,826 |
| ENT_20180622 | 23,333 | 7,572 | 1,973 | 384 | 868,880 | 98,293 |
| No MPEG | | | | | | |
| ENT_20180622 | 40,667 | 22,053 | 3,502 | 1,709 | 1,466,954 | 838,299 |
| High Chitosan | | | | | | |
| ENT_20180622 | 16,000 | 1,732 | 1,307 | 138 | 582,700 | 19,603 |
| No Chitosan | | | | | | |

The swell force characteristics of each formulation were also assessed. As discussed above, the swell force characteristics of the Baseline Formulation are as set out in Example 1B above.

With respect to the No MPEG Acrylate Formulation, FIG. 13L is a plot of the force exerted over time, to characterize the Radial Force of the samples, and Table 4L summarizes the Radial Force characterizations, including the Peak Swell Force and Impulse values.

TABLE 4L

| Start Batch Hydrogel | Impulse @ | Peak |
|---|---|---|
| Fixed Distance Swell- | 5 min (g*s) | Force (g) |
| 20180920_NoMPEG_swell1 | 35,093 | 126 |
| 20180920_NoMPEG_swell2 | 27,303 | 97 |
| 20180920_NoMPEG_swell3 | 38,757 | 145 |
| Average: | 33,718 | 122 |
| S.D. | 5,849 | 24 |

With respect to the High Chitosan Formulation, FIG. 13M is a plot of the force exerted over time, to characterize the Radial Force of the samples, and Table 4M summarizes the Radial Force characterizations, including the Peak Swell Force and Impulse values.

TABLE 4M

| Start Batch Hydrogel | Impulse @ | Peak |
|---|---|---|
| Fixed Distance Swell- | 5 min (g*s) | Force (g) |
| High-chitosan-swell-1 | 8,456 | 25 |
| High-chitosan-swell-2 | 12,254 | 42 |
| High-chitosan-swell-3 | 9,691 | 38 |
| Average: | 10,134 | 35 |
| S.D. | 1,937 | 9 |

With respect to the No Chitosan Formulation, FIG. 13N is a plot of the force exerted over time, to characterize the Radial Force of the samples, and Table 4N summarizes the Radial Force characterizations, including the Peak Swell Force and Impulse values.

TABLE 4N

| Start Batch Hydrogel | Impulse @ | Peak |
|---|---|---|
| Fixed Distance Swell- | 5 min (g*s) | Force (g) |
| 20180920_NoChitosan_swell1 | 18,004 | 81 |
| 20180920_NoChitosan_swell2 | 27,213 | 106 |
| 20180920_NoChitosan_swell3 | 26,204 | 112 |
| Average: | 23,807 | 100 |
| S.D. | 5,051 | 17 |

The radial swelling strength results for the formulations are summarized in Table 40 below, as well as FIGS. 130 and 13P.

TABLE 4O

| | Impulse @ | | Peak | |
|---|---|---|---|---|
| Formulation | 5 min (g*s) | Stdev | Force (g) | Stdev |
| ENT_20180622 | 21,880 | 4,491 | 84 | 19 |
| ENT_20180622 No MPEG | 33,718 | 5,849 | 122 | 24 |
| ENT_20180622 High Chitosan | 10,134 | 1,937 | 35 | 9 |
| ENT_20180622 No Chitosan | 23,807 | 5,051 | 100 | 17 |

As can be seen from the results herein, increasing the amount of chitosan can be seen to decrease the final Swelling Ratio at 10 minutes and the Swell Ratio Percentage achieved at one minute. Excluding chitosan completely resulted in an increased the Final Swelling Ratio at 10 minutes, but decreased the Swell Ratio Percentage at one minute. Excluding MPEG acrylate had little effect on the Swelling Ratio or Swell Ratio Percentage at one minute. Table 4P summarizes the results below.

TABLE 4P
Swell Kinetics

| | Swell | Swell % at |
|---|---|---|
| Formulation | Ratio Q | 1 Minute |
| ENT_20180522 | 60.5 | 73 |
| ENT_20180622 No MPEG | 60.7 | 71 |
| ENT_20180622 High Chitosan | 20.3 | 52 |
| ENT_20180522 No Chitosan | 86 | 46 |

With respect to Compressive Strength, the results show that removing MPEG Acrylate from the formulation significantly reduces the yield point, peak force under compression, and energy absorption ability of the SPH. Increasing the amount of chitosan increases these characteristics on average, though significantly increases the variability of the SPH's mechanical properties. Excluding chitosan completely significantly reduces all mechanical properties of the SPH. Interestingly, the results for the Baseline Formulation show that similar Compressive Strength values to the High Chitosan Formulation can be obtained for formulations comprising MPEG Acrylate, with significantly less chitosan added, while also allowing for a good Swelling Ratio and Swell Ratio Percentage as shown in Table 40 above. The Compressive Force results are summarized in Table 4Q below.

TABLE 4Q
Compression Strength (N = 3)

| | Approximate | | Peak Force Under | | Energy Absorption @ | |
|---|---|---|---|---|---|---|
| Formulation | Yield Point (Pa) | Stdev | Compression (g) | Stdev | 95% Strain (J/m^{∧}3) | Stdev |
| ENT_20180622 | 43,333 | 5,774 | 2,737 | 108 | 1,111,580 | 141,826 |
| ENT_20180622 | 23,333 | 7,572 | 1,973 | 384 | 868,880 | 98,293 |
| No MPEG | | | | | | |
| ENT_20180622 | 40,667 | 22,053 | 3,502 | 1,709 | 1,466,954 | 838,299 |
| High Chitosan | | | | | | |
| ENT_20180622 | 16,000 | 1,732 | 1,307 | 138 | 582,700 | 19,603 |
| No Chitosan | | | | | | |

With respect to the Radial Force or swelling force, the results show that removing MPEG from the formulation slightly increases the swelling force of the SPH compared to the base formulation. Increasing the amount of chitosan from about 0.5% to 2.25% significantly inhibits the swell force of the SPH while excluding it completely slightly increases the swell force. The Radial Force results are summarized in Table 4R below.

TABLE 4R

| | Impulse @ | | Peak | |
|---|---|---|---|---|
| Formulation | 5 min (g*s) | Stdev | Force (g) | Stdev |
| ENT_20180622 | 21,880 | 4,491 | 84 | 19 |
| ENT_20180622 No MPEG | 33,718 | 5,849 | 122 | 24 |
| ENT_20180622 High Chitosan | 10,134 | 1,937 | 35 | 9 |
| ENT_20180622 No Chitosan | 23,807 | 5,051 | 100 | 17 |

Finally, referring to FIGS. 13Q-13R, it can be seen that the Baseline Formulation produced a uniform and regular structure with good porosity (FIG. 13Q), whereas the No MPEG Acrylate Formulation provided a less uniform structure with poor overall structure (FIG. 13R).

### Example 5

In this Example, SPH compositions were prepared according to the methods described in Examples 1A-2 above, and properties were compared. The formulations included: Formulation 1, the formulation as prepared in Example 1B, Formulation 2, having higher amounts of chitosan than Formulation 1; Formulation 3, having chitosan but a lower % solids than Formulation 1; Formulation 4, having similar solids to Formulation 1 but no chitosan; Formulation 5, having low solids and no chitosan; Formulation 6, having less cross-linker and solids that in Formulation 1, and Formulation 7, corresponding to the cationic SPH of Example 2. Tables 5A and 5B below summarizes the composition/results for each formulation.

TABLE 5A

| | | | Peak Force | Yield | Impulse |
|---|---|---|---|---|---|
| Formulation | % | % | Compression | Point | at 5 min |
| No. | solids | chitosan | (g) | (Pa) | (g*s) |
| 1 | 52.6 | 0.46 | 2,737 | 43,333 | 21,181 |
| 2 | 47.5 | 0.82 | 1,762 | 16,167 | 16,981 |
| 3 | 36.2 | 0.31 | 1,368 | 14,833 | 29,768 |
| 4 | 52.5 | — | 1,384 | 11,166 | 11,926 |
| 5 | 36.0 | — | 825 | 10,333 | 20,779 |
| 6 | 42.7 | 0.37 | 2,016 | 25,000 | 53,951 |
| 7 | 54.0 | — | 4,392 | 58,333 | 47,270 |

TABLE 5B

| | Peak | Max | | | |
|---|---|---|---|---|---|
| | Swell | Swelling | Swell Ratio | | |
| Formulation | Force | Ratio (at | Percentage | Capsule | |
| No. | (g) | 10 mins) | at 1 min | Escape | Comments |
| 1 | 84 | 61 | 73 | yes | Excellent |
| | | | | | properties |
| 2 | 68 | 73 | 48 | yes | Viscous |
| | | | | | formulation |
| 3 | 113 | 79 | 77 | yes | Decreased |
| | | | | | mechanical |
| | | | | | strength |
| | | | | | compared to |
| | | | | | higher % |
| | | | | | solids and |
| | | | | | chitosan |
| | | | | | formulations |
| 4 | 46 | 77 | 84 | yes | Reduced |
| | | | | | mechanical |
| | | | | | strength and |
| | | | | | swell force |
| | | | | | compared to |
| | | | | | chitosan |
| | | | | | containing |
| | | | | | compositions |
| 5 | 85 | 174 | 69 | yes | High swell |
| | | | | | ratio but |
| | | | | | reduced |
| | | | | | strength |
| | | | | | compared to |
| | | | | | chitosan- |
| | | | | | containing |
| | | | | | formulations |
| 6 | 252 | 93 | 33 | yes | High swell |
| | | | | | ratio but |
| | | | | | reduced |
| | | | | | compressive |
| | | | | | strength |
| | | | | | compared to |
| | | | | | higher cross- |
| | | | | | linking |
| | | | | | compositions. |
| 7 | 201 | 51 | 63 | yes | Excellent |
| | | | | | strength |
| | | | | | properties |
| | | | | | with |
| | | | | | somewhat |
| | | | | | lower swell |
| | | | | | ratio |

### INCORPORATION BY REFERENCE

All patents and patent application publications mentioned herein, are hereby incorporated by reference in their entirety for all purposes as if each individual patent and/or patent application publication was specifically and individually incorporated by reference. In case of conflict, the instant application, including any definitions herein, will control.

### EQUIVALENTS

While specific embodiments have been discussed, the above specification is illustrative and not restrictive. Many variations will become apparent to those skilled in the art upon review of this specification. The full scope of the embodiments should be determined by reference to the claims, along with their full scope of equivalents, and the specification, along with such variations.

What is claimed is:

1. A pharmaceutically acceptable oral dosage form for delivery of an active agent to an intestinal site, the dosage form comprising:
a delivery structure comprising:
a monolithic body of super porous hydrogel (SPH) material, the monolithic body having an exterior surface; and
one or more active agent delivery regions to deliver the active agent, the one or more active agent delivery regions being located at the exterior surface of the monolithic body; and
a protective coating covering at least a portion of the delivery structure,
wherein at least 10 wt % of the active agent contained in the oral dosage form is located in the one or more active agent delivery regions at the exterior surface of the monolithic body.

2. A pharmaceutically acceptable oral dosage form for delivery of an active agent to an intestinal site, the dosage form comprising:
a delivery structure comprising:
a monolithic body of super porous hydrogel (SPH) material, the monolithic body having an exterior surface; and
one or more active agent delivery regions to deliver the active agent, the one or more active agent delivery regions being located at the exterior surface of the monolithic body; and
a protective coating covering at least a portion of the delivery structure,
wherein the SPH material comprises a porous cross-linked polymeric structure comprising a crosslinked polymer matrix having a repeat structure of monomers comprising ionically charged chemical groups, about an ionically charged structural support polymer comprising ionically charged chemical groups, the ionically charged structural support polymer comprising chitosan and having a molecular weight of at least 50,000 g/mol,
wherein at least some of the ionically charged groups of the crosslinked polymer matrix are ion-paired with the ionically charged groups of ionically charged structural support polymer,
wherein each of the ionically charged chemical groups of the ionically charged structural support polymer each have an ionic charge that is the opposite of that of a charge of the ionically charged chemical groups of the repeat structure of the cross-linked polymer matrix, and
wherein the SPH material comprises a Maximum Swell Ratio of at least 20, exhibits a Swell Ratio Percentage of at least 30% of the Maximum Swell Ratio in a time interval of 60 seconds or less, and comprises a Compressive Strength as measured by Yield Point of at least 5,000 Pa.

3. The dosage form according to any preceding claim, wherein at least 20 wt %, at least 30 wt %, at least 50 wt %, at least 60 wt %, at least 70 wt %, at least 80 wt %, at least 90 wt %, at least 95 wt %, at least 98 wt % and/or at least 99 wt % of the active agent contained in the oral dosage form is located in one or more active agent delivery regions at the exterior surface of the monolithic body.

4. The dosage form according to any preceding claim, wherein the monolithic body comprises first and second ends, and a side surface extending between first and second ends.

5. The dosage form according to any preceding claim, wherein the monolithic body comprises a longitudinal axis L extending between first and second ends of the body, and wherein a ratio of a maximum length of the body, as measured according to a maximum distance between the first and second ends in the longitudinal direction, to a maximum width of the body, as measured according to a maximum distance between opposing sides of the side surface in a direction orthogonal to the longitudinal direction, is at least 1.25:1, such as at least 1.5:1, at least 1.75:1, at least 2:1, at least 2.5:1, and/or at least 3:1.

6. The dosage form according to any preceding claim, wherein the side surface comprises a cylindrically-shaped side surface.

7. The dosage form according to any preceding claim, wherein the side surface comprises rectangular prism-shaped side surface.

8. The dosage form according to any preceding claim, wherein the side surface comprises a substantially planar region extending at least partly along the longitudinal axis of the monolithic body, and optionally extending between the first and second opposing ends of the monolithic body.

9. The dosage form according to any preceding claim, wherein the one or more active agent delivery regions are located on the side surface.

10. The dosage form according to any preceding claim, wherein the one or more active agent delivery regions are located on a cylindrically shaped side surface.

11. The dosage form according to any preceding claim, wherein the one or more active agent regions are located on a substantially planar region of the side surface.

12. The dosage form according to any of claims 1-5, wherein the one or more active agent delivery regions are located at surfaces of the first and second ends.

13. The dosage form according to any preceding claim, wherein the one or more active agent delivery regions extend across at least 10%, at least 20%, at least 30%, at least 50%, at least 60%, at least 70%, at least 80%, at least 90%, at least 95%, at least 98% and/or at least 99% of the exterior surface of the monolithic body.

14. The dosage form according to any preceding claim, wherein the one or more active agent delivery regions comprise particles and/or granules of active agent composition containing the active agent disposed on the exterior surface.

15. The dosage form according to claim 14, wherein the particles and/or granules are adhered to the exterior surface via at least one of frictional forces and an adhering agent.

16. The dosage form according to any of claims 14-15, wherein the particles and/or granules have an average particle size in a range of from 1 micron to 100 microns.

17. The dosage form according to any of claims 14-16, wherein at least 80%, 90%, 95%, and/or 99% of the particles and/or granules have a diameter size in a range from 1 micron to 100 microns.

18. The dosage form according to any of claims 14-17, wherein the particles and/or granules of active agent composition further comprise a permeation enhancer.

19. The dosage form according to any of claims 14-18, wherein the particles and/or granules of active agent composition comprise from 0 wt % to 85 wt % permeation enhancer.

20. The dosage form according to any of claims 14-19, wherein the particles and/or granules of active agent composition are formed by compressing the active agent and optionally one or more permeation enhancers and binder into a tablet, and crushing the tablet to form the particles and/or granules.

21. The dosage form according to any of claims 14-20, wherein the particles and/or granules of active agent are disposed on the elongate side surface of the monolithic body.

22. The dosage form according to any of claims 14-21, wherein the particles and/or granules of active agent comprise at least 10 wt %, at least 20 wt %, at least 30 wt %, at least 50 wt %, at least 60 wt %, at least 70 wt %, at least 80 wt %, at least 90 wt %, at least 95 wt %, at least 98 wt % and/or at least 99 wt % of the active agent contained in the dosage form.

23. The dosage form according to any of claims 1-13, wherein the one or more active agent delivery regions comprise one or more compressed tablets having the active agent, the one or more compressed tablets being affixed to the exterior surface of the monolithic body.

24. The dosage form according to claim 23 wherein the monolithic body comprises first and second longitudinal ends, and an elongate surface extending between the first and second ends, and wherein the one or more compressed tablets are affixed to the elongate surface.

25. The dosage form according to any of claims 23-24, wherein the one or more compressed tablets are affixed to one or more first and second longitudinal ends of the monolithic body.

26. The dosage form according to any of claims 23-25, wherein the one or more compressed tablets further comprise a permeation enhancer.

27. The dosage form according to any of claims 23-26, wherein the one or more compressed tablets are affixed to the exterior surface of the monolithic body such that they extend across at least 20%, at least 30%, at least 40%, at least 50%, at least 60%, at least 70%, at least 80%, at least 90%, at least 95%, at least 98%, and/or at least 99% of the exterior surface of the monolithic body.

28. The dosage form according to any of claims 23-27, wherein the one or more compressed tablets are affixed to the exterior surface of the monolithic body by exerting a compressive force to compress the one or more tablets against and/or into the exterior surface.

29. The dosage form according to any of claims 23-28, wherein the one or more compressed tablets are affixed to the exterior surface of the monolithic body by an adhesive that adheres a surface of one or more of the compressed tablets to the exterior surface of the monolithic body.

30. The dosage form according to any of claims 23-29, wherein the one or more compressed tablets are affixed at opposing surface portions of the exterior surface.

31. The dosage form according to any of claims 1-13, wherein the one or more active agent delivery regions comprises a coating containing the active agent that is formed across at least a portion of the exterior surface of the monolithic body.

32. The dosage form according to claim 31, wherein the coating containing the active agent extends across at least 25%, at least 30%, at least 50%, at least 60%, at least 70%, at least 80%, at least 90%, at least 95%, at least 98%, and/or at least 99% of the exterior surface of the monolithic body.

33. The dosage form according to any of claims 31-32, wherein the coating at least partially permeates through the exterior surface at least partially into the interior volume of the monolithic body.

34. The dosage form according to any of claims 31-33, wherein the coating comprises at least 20%, at least 30%, at least 50%, at least 60%, at least 70%, at least 80%, at least 90%, at least 95%, at least 98% and/or at least 99% of the active agent contained in the dosage form.

35. The dosage form according to any of claims 31-34, wherein the coating further comprises a permeation enhancer.

36. The dosage form according to any of claims 31-35, wherein the coating is formed by spray coating of a solution comprising the active agent, and optionally permeation enhancer, onto at least a portion of the exterior surface.

37. The dosage form according to any of claims 1-13, wherein the one or more active agent delivery regions comprises one or more biodegradable films comprising the active agent, formed on at least a portion of the exterior surface.

38. The dosage form according to claim 37, wherein the biodegradable film comprises any one or more of proteins, polysaccharides, carbohydrates, gums, polypeptides, and lipids.

39. The dosage form according to any of claim 37 or 38, wherein the biodegradable film extends across at least 50%, at least 60%, at least 70%, at least 80%, at least 90%, at least 95%, at least 98%, and/or at least 99% of the exterior surface of the monolithic body.

40. The dosage form according to any of claims 37-39, wherein the monolithic body comprises first and second ends, and an elongate surface extending between the first and second ends, and wherein the biodegradable film covers at least a portion of the elongate surface.

41. The dosage form according to any one of claims 37-40, wherein the active agent is incorporated into the composition of the biodegradable film.

42. The dosage form according to any one of claims 37-41, wherein the active agent is disposed on an outer surface of the biodegradable film.

43. The dosage form according to any one of claims 37-42, wherein the active agent is present in the form of one or more of granules and/or particles, compressed tablet, and/or lipid-containing composition, and is disposed on the outer surface of the biodegradable film.

44. The dosage form according to any of claims 1-13, wherein the active agent is incorporated into a lipid-containing composition and disposed on a portion of the exterior surface of the monolithic body.

45. The dosage form according to claim 44, wherein the lipid-containing composition and active agent are contained in one or more capsules affixed to the exterior surface of the monolithic body.

46. The dosage form according to any of claims 44 and 45, wherein the monolithic body comprises first and second opposing ends, and an elongate surface extending between the first and second opposing ends, and wherein the lipid-containing composition and active agent are provided at one or more of the first and second opposing ends.

47. The dosage form according to any preceding claim, wherein the dosage form comprises a single monolithic body comprising the super porous hydrogel.

48. The dosage form according to any preceding claim, wherein the dosage form comprises a plurality of monolithic bodies comprising the super porous hydrogel.

49. The dosage form according to any preceding claim, wherein the SPH body comprise a diameter of at least 5 mm, at least 6 mm, at least 8 mm, at least 9 mm and/or at least 10 mm.

50. The dosage form according to any preceding claim, wherein the SPH body comprise a mass of at least 50 mg, at least 75 mg and/or at least 100 mg, and no more than 2 g, no more than 1 g and/or no more than 0.5 grams.

51. The dosage form according to any preceding claim, wherein a single monolithic body comprising super porous hydrogel comprises at least 20% by weight, at least 30% by weight, at least 50% by weight, at least 60% by weight, at least 70% by weight, at least 80% by weight, at least 90% by weight, at least 95% by weight, at least 98% by weight, and/or at least 99% by weight of the total amount of super porous hydrogel in the dosage form.

52. The dosage form according to any preceding claim, wherein the monolithic body comprises opposing first and second longitudinal end surfaces, and a side surface extending between the first and second longitudinal end surfaces, the side surface extending about a longitudinal axis of the monolithic body that passes through the opposing first and second longitudinal end surfaces.

53. The dosage form according to claim 52, wherein the side surface comprises a cylindrical side surface.

54. The dosage form according to claim 52, wherein the side surface comprises a rectangular prism-shaped side surface.

55. The dosage form according to claim 52, wherein the side surface comprises a combination of curved and substantially planar surfaces.

56. The dosage form according to any preceding claim, wherein the monolithic body is spherically-shaped.

57. The dosage form according to any preceding claim, wherein the one or more active agent delivery regions further contain at least one permeation enhancer that facilitates permeation of the active agent into tissue in the intestinal region.

58. The dosage form according to claim 57, wherein the active agent delivery regions at the exterior surface of the monolithic body comprise at least about 50%, at least 60%, at least 70%, at least 80%, at least 90%, at least 95%, at least 98% and/or at least 99% of the permeation enhancer contained in the dosage form.

59. The dosage form according to any preceding claim, wherein in a case that the monolithic body comprises a surface indentation or void formed therein that is in connection with the exterior surface, the indentation and/or void has a volume that does not exceed 30%, 20%, 10%, 8%, 7.5%, 7%, 6%, 5%, 3.5%, 3%, 1.5%, 1% and/or 0.5% of the total volume occupied by the monolithic body.

60. The dosage form according to any preceding claim, wherein in a case that the monolithic body comprises one or more surface indentations or voids formed therein in connection with the exterior surface, the one or more indentations and/or voids have a total combined volume that does not exceed 30%, 20%, 10%, 8%, 7.5%, 7%, 6%, 5%, 3.5%, 3%, 1.5%, 1% and/or 0.5% of the total volume occupied by the monolithic body.

61. The dosage form according to any preceding claim, wherein in a case where the monolithic body comprises one or more indentations or voids formed therein, the volume of such void or hole does not exceed 40 mm³, 30 mm³, and/or 20 mm³.

62. The dosage form according to any preceding claim, wherein a total volume of any surface indentations and/or voids connected to the exterior surface and having a volume greater than 40 mm³, 50 mm³, and/or 65 mm³ does not exceed 30%, 20%, 10%, 8%, 5%, 3%, 1.5%, 1% and/or 0.5% of the total volume occupied by the monolithic body.

63. The dosage form according to any preceding claim, wherein an amount of active agent present in any surface indentation and/or void connected to the exterior surface and having a volume greater than 40 mm³, 50 mm³, and/or 65 mm³ is less than 50 wt %, less than 40 wt %, less than 30 wt %, less than 20 wt %, less than 10 wt %, less than 8 wt %, less than 5 wt %, less than 3 wt %, less than 2 wt %, less than 1.5 wt %, less than 1 wt %, less than 0.5 wt %, and/or less than 0.1 wt %.

64. The dosage form according to any preceding claim, wherein the superporous hydrogel comprises an Effective Density in a Dried State of less than 0.9 g/cm³, less than 0.8 g/cm³, less than 0.75 g/cm³, less than 0.6 g/cm³, less than 0.5 g/cm³, less than 0.45 g/cm³, less than 0.3 g/cm³, and/or less than 0.25 g/cm³, and greater than 0.05 g/cm³.

65. The dosage form according to any preceding claim, wherein the super porous hydrogel comprises a 3-dimensional network of hydrophilic polymers.

66. The dosage form according to any preceding claim, wherein the super porous hydrogel comprises a polymeric network formed from any one or more of acrylic acid, acrylamide, sodium acrylate, 2-hydroxyethyl methacrylate, 2-acrylamido-2-methyl-1-propanesulfonic acid, 2-acryloyloxy ethyl trimethylammonium methyl sulfate, 2-hydroxypropyl methacrylate, 3-sulphopropyl acrylate potassium, hydroxyl ethyl methyl acrylate, N-isopropyl acrylamide, acrylonitrile, polyvinyl alcohol, glutaraldehyde, N, N-methylenebisacrylamide, N, N, N, N-tetramethylenediamine, pluronic F127, hydroxyethyl acrylate, diethylene glycol diacrylate, polyethylene glycol acrylate, polyethylene glycol diacrylate, cross-linked sodium carboxymethylcellulose (Ac-Di-Sol), crosslinked sodium starch glycolate (Primojel), crosslinked polyvinylpyrrolidone (crospovidone), Carbopol, sodium alginate, sodium carboxymethylcellulose, chitosan, pectin, or salts thereof.

67. The dosage form according to any preceding claim, wherein the monolithic body of super porous hydrogel material comprises one or more of super porous hydrogel, super porous hydrogel composite and super porous hydrogel hybrid.

68. The dosage form according to any preceding claim, wherein the monolithic body comprises a unitary body of the super porous hydrogel material.

69. The dosage form according to any preceding claim, wherein the monolithic body comprises a Compressive Strength as measured by the Yield Point of at least 5,000, at least 8,000 Pa, at least 10,000 Pa, at least 15,000 Pa, at least 18,000 Pa, at least 20,000 Pa, at least 25,000 Pa, least 30,000 Pa, at least 35,000 Pa, at least 40,000 Pa and/or at least 45,000 Pa, and no more than 100,000.

70. The dosage form according to any preceding claim, wherein the monolithic body comprises a Compressive Strength as measured by the Yield Point in a range of from 8,000 Pa to 100,000 Pa, in a range from 20,000 Pa to 90,000 Pa, and/or in a range from 30,000 Pa to 80,000 Pa.

71. The dosage form according to any preceding claim, wherein the monolithic body comprises a Maximum Swell Ratio of at least 20, at least 25, at least 35, at least 40, at least 45, at least 50, at least 55, at least 60, at least 65, at least 70, at least 75, at least 80, at least 85, at least 90, at least 95, at least 100, at least 115, at least 120, at least 130, at least 140, at least 150, at least 160, at least 170, at least 180, at least 190, at least 200, and/or at least 250.

72. The dosage form according to any preceding claim, wherein the monolithic body comprises a Maximum Swell Ratio that is in a range of from 30 to 100, in a range of from 40 to 80, and/or in a range of from 50 to 75.

73. The dosage form according to any preceding claim, wherein the monolithic body comprises a Swell Ratio Percentage of at least 30%, at least 35%, at least 45%, at least 50%, at least 55%, at least 60% and/or at least 70% of a Maximum Swell Ratio for the SPH material at a time interval of 60 seconds or less.

74. The dosage form according to any preceding claim, wherein the monolithic body comprises Swell Ratio Percentage that is in a range of from 30% to 100%, 40% to 90%, and/or 50% to 80% of a Maximum Swell Ratio for the SPH material at a time interval of 60 seconds or less.

75. The dosage form according to any preceding claim, wherein the SPH body comprises a Radial Swell Force measured at a surface thereof of at least 15 g, at least 25 g, at least 30 g, at least 35 g, at least 40 g, at least 50 g, at least 60 g, at least 75 g, and/or at least 100 g, and less than 1000 g.

76. The dosage form according to any preceding claim, wherein the SPH body comprises a Radial Swell Force measured at a surface that is in a range of from 50 g to 1000 g, and/or in a range of from 70 g to 250 g, and/or in a range of from 75 g to 200 g.

77. The dosage form according to any preceding claim, wherein the dosage form further comprises one or more permeation enhancers selected from the group consisting of sodium deoxycholate, hexylamine, DTAB, sodium lauryl sulfate, sodium caprate, lauroyl carnitine, EDTA, palmitoyl carnitine, PPS, and dimethyl palmitoyl ammonio propanesulfonate, or salts thereof.

78. The dosage form according to any preceding claim, wherein the protective coating comprises a capsule.

79. The dosage form according to any preceding claim, wherein the protective coating comprises an enteric coating.

80. The dosage form according to any preceding claim, wherein the protective coating comprises a capsule coated with an enteric coating.

81. The dosage form according to any preceding claim, comprising an enteric coating that becomes at least partially permeable when exposed to gastric fluid at a pH of from about 5.5 to about 7.5.

82. The dosage form according to any of claims 1-3, wherein the delivery structure comprises a plurality of bodies of SPH material.

83. The dosage form according to any preceding claim, wherein the body of SPH material comprises a plurality of crevices.

84. The dosage form according to any preceding claim, wherein the body of SPH is in a Compressible State and comprises an amount of retained water of at least 2.5%, at least 5%, and/or at least 8%, and no more than 10% by weight of the SPH body.

85. The dosage form according to any preceding claim, wherein a volume of the body of SPH is in a Compressed State having a compressed volume corresponding to less than 90%, less than 80%, less than 75%, less than 60% and/or less than 50% of the body SPH in an Uncompressed State.

86. The dosage form according to any preceding claim, where the body of SPH in the Compressed State retains a Swell Speed in which a Swell Ratio Percentage of at least 30%, at least 35%, at least 45%, at least 50%, at least 55%, at least 60% and/or at least 70% of a Maximum Swell Ratio for the SPH body is achieved at a time interval of 60 seconds or less.

87. The dosage form according to any preceding claim, wherein the body of SPH is in a Compressed State and exhibits a Volume Swell Ratio of at least 20, at least 30, at least 40, at least 50, at least 60, at least 70 and/or at least 80.

87. The dosage form according to any preceding claim, wherein the body of SPH is in a Compressed State and exhibits a Volume Swell Ratio that is at least 2 times, at least 3 times, at least 4 times and/or at least 5 times a Volume Swell Ratio of the body of SPH material in an Uncompressed State.

89. A method of forming a super-porous hydrogel (SPH) material for use with the SPH body of claim 1 or 2, the method comprising:
forming a polymerization mixture by combining (i) a structural support material comprising at least one ionically charged structural support polymer having a molecular weight of at least 50,000 g/mol, the ionically charged structural support polymer having a plurality of ionically charged chemical groups, (ii) a monomer material comprising at least one ionically charged ethylenically-unsaturated monomer, and (iii) at least one cross-linking agent;
forming a foam of the polymerization mixture; and
polymerizing the foam to form a porous crosslinked polymeric structure having ion-pairing between a cross-linked polymer matrix formed by polymerization of the ionically charged ethylenically-unsaturated monomer with the cross-linking agent, and the ionically charged structural support polymer,
wherein each of the ionically charged chemical groups of the ionically charged structural support polymer each have an ionic charge that is the opposite of that of a charge of the ionically charged ethylenically-unsaturated monomer.

90. A super-porous hydrogel (SPH) material for the SPH body of claim 1 or claim 2, comprising:
a porous cross-linked polymeric structure comprising a crosslinked polymer matrix having a repeat structure of monomers comprising ionically charged chemical groups, about an ionically charged structural support polymer comprising ionically charged chemical groups, the ionically charged structural support polymer having a molecular weight of at least 50,000 g/mol,
wherein at least some of the ionically charged groups of the crosslinked polymer matrix are ion-paired with the ionically charged groups of ionically charged structural support polymer, and
wherein each of the ionically charged chemical groups of the ionically charged structural support polymer each have an ionic charge that is the opposite of that of a charge of the ionically charged chemical groups of the repeat structure of the cross-linked polymer matrix.

91. The method and/or SPH material according to any of claims 89-90, wherein the SPH material comprises a Maximum Swell Ratio of at least 20, at least 25, at least 35, at least 40, at least 45, at least 50, at least 55, at least 60, at least 65, at least 70, at least 75, at least 80, at least 85, at least 90, at least 95, at least 100, at least 115, at least 120, at least 130, at least 140, at least 150, at least 160, at least 170, at least 180, at least 190, at least 200, and/or at least 250.

92. The method and/or SPH material according to any of claims 89-91, wherein the SPH material comprises a Maximum Swell Ratio in a range of from 30 to 1000, and/or in a range of from 40 to 80, and/or in a range of from 50 to 75.

93. The method and/or SPH material according to any of claims 89-92, wherein the SPH material comprises a Swell Ratio Percentage of at least 30%, at least 35%, at least 45%, at least 50%, at least 55%, at least 60% and/or at least 70% of a Maximum Swell Ratio for the SPH material at a time interval of 60 seconds or less.

94. The method and/or SPH material according to any of claims 89-93, wherein the Swell Ratio Percentage is in a range of from 30% to 100%, 40 to 90%, and/or 50% to 80% of a Maximum Swell Ratio for the SPH material at a time interval of 60 seconds or less.

95. The method and/or SPH material according to any of claims 89-94, wherein the SPH material comprises a Compressive Strength as measured by the Yield Point of at least 5,000, at least 8,000 Pa, at least 10,000 Pa, at least 15,000 Pa, at least 18,000 Pa, at least 20,000 Pa, at least 25,000 Pa, least 30,000 Pa, at least 35,000 Pa, at least 40,000 Pa and/or at least 45,000 Pa, and no more than 100,000.

96. The method and/or SPH material according to any of claims 89-95, wherein the monolithic body comprises a Compressive Strength as measured by the Yield Point in a range of from 8,000 Pa to 100,000 Pa, in a range from 20,000 Pa to 90,000 Pa, and/or in a range from 30,000 Pa to 80,000 Pa.

97. The method and/or SPH according to any of claims 89-96, wherein the SPH material comprises a Radial Swell Force as measured at a surface thereof of at least 15 g, at least 25 g, at least 30 g, at least 35 g, at least 40 g, at least 50 g, at least 60 g, at least 75 g, and/or at least 100 g, and less than 1000 g.

98. The method and/or SPH according to any of claims 89-97, wherein the SPH material comprises a Radial Swell Force as measured at a surface thereof that is in a range of from 50 g to 1000 g, and/or in a range of from 70 g to 250 g, and/or in a range of from 75 g to 200 g.

99. The method and/or SPH material according to any of claims 89-98, wherein the ionically charged chemical groups of the ethylenically-unsaturated monomer are anionically charged, and the ionically charged chemical groups of the ionically charged structural support polymer are cationically charged.

100. The method and/or SPH material according to any of claims 89-99, wherein the ionically charged chemical groups of the ionically charged ethylenically-unsaturated monomer are cationically charged, and the ionically charged chemical groups of the ionically charged structural support polymer are anionically charged.

101. The method and/or SPH material according to any of claims 89-100, wherein the ionically charged ethylenically-unsaturated monomer comprises any selected from the group consisting of acrylate monomers (salts of (meth)acrylic acid), salts of esters of (meth) acrylic acid, salts of N-alkyl amides of (meth)acrylic acid, sulfopropyl acrylate monomers, PEG acrylate, and 2-(acryloyloxy)ethyl trimethylammonium methyl sulfate, and/or salts thereof.

102. The method and/or SPH material according to any of claims 89-101, wherein the monomer material further comprises non-ionically charged ethylenically-unsaturated monomers, including any selected from the group consisting of acrylamide monomers, acrylamidopropyl monomers, esters of (meth)acrylic acid and their derivatives (2-hydroxyethyl (meth) acrylate, hydroxypropyl(meth) acrylate, butanediol monoacrylate), N-alkyl amides of (meth) acrylic acid, N-vinyl pyrrolidone, (meth)acrylamide derivatives (N-isopropyl acrylamide, N-cyclopropyl (meth)acrylamide, N.N-dimethylaminoethyl acrylate, and 2-acrylamido-2-methyl-1-propanesulfonic acid, and/or salts thereof.

103. The method and/or SPH material according to any of claims 89-102, wherein the monomer material further comprises an acrylate monomer having a polyethylene glycol repeat group of the following formula: 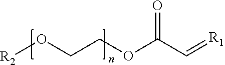
where R₁ and R₂ are each independently hydrocarbyl with 6 carbons or less, or hydrogen, n is on average in a range of from 2 to about 20, or is in a range of from about 5 to about 15, and/or is in a range of from about 8 to 12.

104. The method and/or SPH material according to any of claims 89-103, wherein the monomer material comprises MPEG acrylate.

105. The method and/or SPH material according to any of claims 89-104, wherein the ionically charged structural support material comprises an ionically charged structural support polymer selected from the group consisting of a polysaccharide, chitosan, chitins, alginate, cellulose, cyclodextrin, dextran, gums, lignins, pectins, saponins, deoxyribonucleic acid, ribonucleic acids, polypeptides, protein, albumin, bovine serum albumin, casein, collagen, fibrinogen, gelatin, gliaden, poly amino acids, synthetic polymers, (meth) acrylamide polymer, (meth)acrylic acid polymer, (meth) acrylate polymer, acrylonitrile, ethylene polymers, ethylene glycol polymers, ethyleneimine polymers, ethyleneoxide polymers, styrene sulfonate polymers, vinyl acetate polymers, vinyl alcohol polymers, vinyl chloride polymers, and vinylpyrrolidone polymers and/or derivatives, salts, and/or homo or copolymers thereof.

106. The method and/or SPH material according to any of claims 89-105, wherein the ionically charged structural support polymer comprises a molecular weight of at least 55,000 g/mol MW, at least 65,000 g/mol MW, at least 80,000 g/mol MW, at least 100,000 g/mol MW, at least 125,000 g/mol MW, at least 150,000 g/mol MW, at least 175,000 g/mol MW, at least 200,000 g/mol MW, and/or at least 225,000 g/mol MW.

107. The method and/or SPH material according to any of claims 89-106, wherein the ionically charged structural support polymer has a molecular weight in the range of from 50,000 g/mol MW to 250,000 g/mol MW.

108. The method and/or SPH according to any of claims 89-107, wherein the SPH material comprises an Effective Density in a Dried State of less than 0.9 g/cm³, less than 0.8 g/cm³, less than 0.75 g/cm³, less than 0.6 g/cm³, less than 0.5 g/cm³, less than 0.45 g/cm³, less than 0.3 g/cm³, and/or less than 0.25 g/cm³, and greater than 0.05 g/cm³.

109. The method and/or SPH according to any of claims 89-108, wherein the crosslinking agent comprise at least one selected from the group consisting of N,N′-methylene bisacrylamide, N,N′-methylene bisacrylamide, (poly)ethylene glycol di(meth)acrylate, ethylene glycol diglycidyl ether, glycidyl methacrylate, polyamidoamine epichlorohydrin, and N,N′-diallyltartardiamide.

110. The method and/or SPH material according to any of claims 89-109, wherein the polymerization mixture that is polymerized to form the SPH material comprises at least 1% by weight, at least 5% by weight, at least 8% by weight, and/or at least 10% by weight of the monomer material comprising at the least one ionically charged ethylenically-unsaturated monomer, and no more than 35% by weight, 25% by weight, 18% by weight and/or 15% by weight of the monomer material comprising at the least one ionically charged ethylenically-unsaturated monomer.

111. The method and/or SPH material according to any of claims 89-110, wherein the monomer material comprising at the least one ionically charged ethylenically-unsaturated monomer is acrylic acid, and/or a salt thereof.

112. The method and/or SPH material according to any of claims 89-111, wherein the polymerization mixture that is polymerized to form the SPH material comprises at least 0.25%, at least 0.3% by weight, at least 0.45% by weight, and/or at least 0.5% by weight of the structural support material comprising the at least one ionically charged structural support polymer, and no more than 1% by weight, no more than 0.90% by weight, no more than 0.85% by weight and/or no more than 0.75% by weight of the structural support material comprising the at least one ionically charged structural support polymer.

113. The method and/or SPH material according to any of claims 89-112, wherein the at least one ionically charged structural support polymer is chitosan and/or a salt thereof.

114. The method and/or SPH material according to any of claims 89-113, wherein the polymerization mixture that is polymerized to form the SPH material comprises at least 0.001% by weight, at least 0.01% by weight, at least 0.1% by weight, and/or at least 0.5% by weight of the cross-linking agent, and no more than 1% by weight, 0.8% by weight, 0.7% by weight and/or 6% by weight of the cross-linking agent.

115. The method and/or SPH material according to any of claims 89-114, wherein the cross-linking agent is methylene bisacrylamide.

116. The method and/or SPH material according to any of claims 89-115, wherein the polymerization mixture that is polymerized to form the SPH material comprises at least 1% by weight, at least 5% by weight, at least 15% by weight, and/or at least 25% by weight of a non-ionically charged ethylenically unsaturated monomer, and no more than 50% by weight, 45% by weight, 35% by weight and/or 30% by weight of the non-ionically charged ethylenically unsaturated monomer.

117. The method and/or SPH material according to any of claims 89-116, wherein the non-ionically charged ethylenically unsaturated monomer comprises acrylamide.

118. The method and/or SPH material according to any of claims 89-117, wherein the polymerization mixture that is polymerized to form the SPH material comprises at least 1% by weight, at least 5% by weight, at least 8% by weight, and/or at least 10% by weight of an acrylate monomer having a polyethylene glycol repeat group, and no more than 35% by weight, 30% by weight, 20% by weight and/or 15% weight of the acrylate monomer having a polyethylene glycol repeat group.

119. The method and/or SPH material according to any of claims 89-118, wherein the acrylate monomer having the polyethylene glycol repeat group comprises MPEG acrylate.

120. The method and/or SPH material according to any of claims 89-119, wherein the polymerization mixture that is polymerized to form the SPH material comprises a combined amount of the monomer material, structural support material, and at least one cross-linking agent, that is greater than 25%, 30%, 35%, 40% and/or 50% by weight of the total weight of the polymerization mixture, and no more than 90%, no more than 80% and/or no more than 75% by weight of the total weight of the polymerization mixture.

121. The method and/or SPH material according to any of claims 89-120, wherein the SPH material is at least partially dried in a humidified environment comprising an environmental humidity of at least 50%, at least 65%, and/or at least 75%.

122. The method and/or SPH material according to any of claims 89-121, wherein the SPH material is in a Compressible State and comprises an amount of retained water of at least 2.5%, at least 5%, and/or at least 8%, and no more than 10% by weight of the SPH material.

123. The method and/or SPH material according to any of claims 89-122, wherein a volume of the SPH material is in a Compressed State having a compressed volume corresponding to less than 90%, less than 80%, less than 75%, less than 60% and/or less than 50% of the SPH material in an Uncompressed State.

124. The method and/or SPH material according to claim 123, where the SPH material in the Compressed State retains a Swell Speed in which a Swell Ratio Percentage of at least 30%, at least 35%, at least 45%, at least 50%, at least 55%, at least 60% and/or at least 70% of a Maximum Swell Ratio for the SPH material is achieved at a time interval of 60 seconds or less.

125. The method and/or SPH material according to any of claims 89-124, wherein the SPH material in the Compressed State exhibits a Volume Swell Ratio of at least 20, at least 30, at least 40, at least 50, at least 60, at least 70 and/or at least 80.

126. The method and/or SPH material according to any of claims 89-125, wherein the SPH material in the Compressed State exhibits a Volume Swell Ratio that is at least 2 times, at least 3 times, at least 4 times and/or at least 5 times a Volume Swell Ratio of the SPH material in an Uncompressed State.

127. The method and/or SPH material according to any of claims 89-126, wherein the SPH material is an elastic material.

128. The method and/or SPH material according to any of claims 89-127, wherein the SPH material comprises one or more crevices formed therein.

129. An SPH material formed by a method according to any of claims 89 and 91-128.

130. A method of forming a super-porous hydrogel (SPH) material for the SPH body of any preceding claim, the method comprising:
forming a polymerization mixture by combining (i) a monomer material comprising at least one cationically charged ethylenically-unsaturated monomer, and optionally at least one non-ionically charged ethylenically unsaturated monomer, and (ii) at least one cross-linking agent;
forming a foam of the polymerization mixture; and
polymerizing the foam to form a porous crosslinked polymeric structure formed by polymerization of the cationically charged ethylenically-unsaturated monomer with the cross-linking agent, and optionally with the neutral ethylenically unsaturated monomer,
wherein the porous crosslinked polymeric structure comprises a Maximum Swell Ratio of at least 20, and a Compressive Strength as measured by the Yield Point of at least 5000 Pascals.

131. A super-porous hydrogel (SPH) material for the SPH body of any preceding claims, comprising:
a porous cross-linked polymeric structure comprising a crosslinked polymer matrix having a repeat structure of monomer residues obtained from cationically charged ethylenically-unsaturated monomers, and optionally monomer residues obtained from non-ionically charged ethylenically-unsaturated monomers,
wherein the porous cross-linked polymeric structure comprises a Maximum Swell Ratio of at least 20, and a Compressive Strength as measured by the Yield Point of at least 5000 Pascals.

132. The method and/or SPH material according to any of claims 130-131, wherein the SPH material comprises a Maximum Swell Ratio of at least 25, at least 35, at least 40, at least 45, at least 50, at least 55, at least 60, at least 65, at least 70, at least 75, at least 80, at least 85, at least 90, at least 95, at least 100, at least 115, at least 120, at least 130, at least 140, at least 150, at least 160, at least 170, at least 180, at least 190, at least 200, and/or at least 250.

133. The method and/or SPH material according to any of claims 130-132, wherein the SPH material comprises a Maximum Swell Ratio in a range of from 30 to 1000, and/or in a range of from 40 to 80, and/or in a range of from 50 to 75.

134. The method and/or SPH material according to any of claims 130-133, wherein the SPH material comprises a Swell Ratio Percentage of at least 30%, at least 35%, at least 45%, at least 50%, at least 55%, at least 60% and/or at least 70% of a Maximum Swell Ratio for the SPH material at a time interval of 60 seconds or less.

135. The method and/or SPH material according to any of claims 130-134, wherein the SPH material comprises a Swell Ratio Percentage in a range of from 30% to 100%, 40% to 90%, and/or 50% to 80% of a Maximum Swell Ratio at a time interval of 60 seconds or less.

136. The method and/or SPH material according to any of claims 130-135, wherein the SPH material comprises a Compressive Strength as measured by the Yield Point of 8,000 Pa, at least 10,000 Pa, at least 15,000 Pa, at least 18,000 Pa, at least 20,000 Pa, at least 25,000 Pa, at least 30,000 Pa, at least 35,000 Pa, at least 40,000 Pa and/or at least 45,000 Pa, and no more than 100,000.

137. The method and/or SPH material according to any of claims 130-136, wherein the monolithic body comprises a Compressive Strength as measured by the Yield Point in a range of from 8,000 Pa to 100,000 Pa, in a range from 20,000 Pa to 90,000 Pa, and/or in a range from 30,000 Pa to 80,000 Pa.

138. The method and/or SPH according to any of claims 130-137, wherein the SPH material comprises a Radial Swell Force as measured at a surface thereof of at least 15 g, at least 25 g, at least 30 g, at least 35 g, at least 40 g, at least 50 g, at least 60 g, at least 75 g, and/or at least 100 g, and less than 1000 g.

139. The method and/or SPH according to any of claims 130-138, wherein the SPH material comprises a Radial Swell Force as measured at a surface thereof that is in a range of from 50 g to 1000 g, and/or in a range of from 70 g to 250 g, and/or in a range of from 75 g to 200 g.

140. The method and/or SPH material according to any of claims 130-139, wherein the cationically charged ethylenically-unsaturated monomer comprises any selected from the group consisting of 3-(amino)propyl methacrylamide, 3-(dimethylamino)propyle-methacrylamide, 3-(trimethylammonium)propyl-methacrylamide, and/or salts thereof.

141. The method and/or SPH material according to any of claims 130-140, wherein the monomer material further comprises non-ionically charged ethylenically-unsaturated monomers, including any selected from the group consisting of acrylamide monomers, acrylamidopropyl monomers, esters of (meth)acrylic acid and their derivatives (2-hydroxyethyl (meth) acrylate, hydroxypropyl(meth) acrylate, butanediol monoacrylate), N-alkyl amides of (meth) acrylic acid, N-vinyl pyrrolidone, (meth)acrylamide derivatives (N-isopropyl acrylamide, N-cyclopropyl (meth)acrylamide, N.N-dimethylaminoethyl acrylate, and 2-acrylamido-2-methyl-1-propanesulfonic acid.

142. The method and/or SPH material according to any of claims 130-141, wherein the monomer material further comprises an acrylate monomer having a polyethylene glycol repeat group of the following formula: 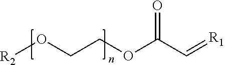
where R₁ and R₂ are each independently hydrocarbyl with 6 carbons or less, or hydrogen, n is on average in a range of from 2 to about 20, or is in a range of from about 5 to about 15, and/or is in a range of from about 8 to 12.

143. The method and/or SPH material according to any of claims 130-142, wherein the monomer material comprises MPEG acrylate.

144. The method and/or SPH according to any of claims 130-143, wherein the SPH material comprises an Effective Density in a Dried State of less than 0.9 g/cm³, less than 0.8 g/cm³, less than 0.75 g/cm³, less than 0.6 g/cm³, less than 0.5 g/cm³, less than 0.45 g/cm³, less than 0.3 g/cm³, and/or less than 0.25 g/cm³, and greater than 0.05 g/cm³.

145. The method and/or SPH according to any of claims 130-144, wherein the crosslinking agent comprise at least one selected from the group consisting of N,N′-methylene bisacrylamide, N,N′-methylene bisacrylamide, (poly)ethylene glycol di(meth)acrylate, ethylene glycol diglycidyl ether, glycidyl methacrylate, polyamidoamine epichlorohydrin, and N,N′-diallyltartardiamide.

146. The method and/or SPH material according to any of claims 130-145, wherein the polymerization mixture that is polymerized to form the SPH material comprises at least 1% by weight, at least 5% by weight, at least 8% by weight, and/or at least 10% by weight of the monomer material comprising at least one cationically charged ethylenically-unsaturated monomer, and no more than 35% by weight, 30% by weight, 25% by weight and/or 20% by weight of the monomer material comprising at least one cationically charged ethylenically-unsaturated monomer.

147. The method and/or SPH material according to any of claims 130-146, wherein the monomer material comprising at the least one cationically charged ethylenically-unsaturated monomer is (3-acrylamidopropyl)trimethylammonium, and/or a salt thereof.

148. The method and/or SPH material according to any of claims 130-147, wherein the polymerization mixture that is polymerized to form the SPH material comprises at least 0.001% by weight, at least 0.01% by weight, at least 0.1% by weight, and/or at least 0.5% by weight of the cross-linking agent, and no more than 1% by weight, 0.8% by weight, 0.7% by weight and/or 6% by weight of the cross-linking agent.

149. The method and/or SPH material according to any of claims 130-148, wherein the cross-linking agent is methylene bisacrylamide.

150. The method and/or SPH material according to any of claims 130-149, wherein the polymerization mixture that is polymerized to form the SPH material comprises at least 1% by weight, at least 5% by weight, at least 15% by weight, and/or at least 25% by weight of a non-ionically charged ethylenically unsaturated monomer, and no more than 50% by weight, 45% by weight, 35% by weight and/or 30% by weight of the non-ionically charged ethylenically unsaturated monomer.

151. The method and/or SPH material according to any of claims 130-150, wherein the non-ionically charged ethylenically unsaturated monomer comprises acrylamide.

152. The method and/or SPH material according to any of claims 130-151, wherein the polymerization mixture that is polymerized to form the SPH material comprises at least 1% by weight, at least 5% by weight, at least 8% by weight, and/or at least 10% by weight of an acrylate monomer having a polyethylene glycol repeat group, and no more than 35% by weight, 30% by weight, 20% by weight and/or 15% by weight of the acrylate monomer having a polyethylene glycol repeat group.

153. The method and/or SPH material according to any of claims 130-152, wherein the acrylate monomer having the polyethylene glycol repeat group comprises MPEG acrylate.

154. The method and/or SPH material according to any of claims 130-153, wherein the polymerization mixture that is polymerized to form the SPH material comprises a combined amount of the monomer material and at least one cross-linking agent, that is greater than 25%, 30%, 35%, 40% and/or 50% by weight of the total weight of the polymerization mixture, and no more than 90%, no more than 80% and/or no more than 75% by weight of the total weight of the polymerization mixture.

155. The method and/or SPH material according to any of claims 130-154, wherein the SPH material is at least partially dried in a humidified environment comprising an environmental humidity of at least 50%, at least 65%, and/or at least 75%.

156. The method and/or SPH material according to any of claims 130-155, wherein the SPH material is in a Compressible State and comprises an amount of retained water of at least 2.5%, at least 5%, and/or at least 8%, and no more than 10% by weight of the SPH material.

157. The method and/or SPH material according to any of claims 129-156, wherein a volume of the SPH material is in a Compressed State having a compressed volume corresponding to less than 90%, less than 80%, less than 75%, less than 60% and/or less than 50% of the SPH material in an Uncompressed State.

158. The method and/or SPH material according to claim 157, where the SPH material in the Compressed State retains a Swell Speed in which a Swell Ratio Percentage of at least 30%, at least 35%, at least 45%, at least 50%, at least 55%, at least 60% and/or at least 70% of a Maximum Swell Ratio for the SPH material is achieved at a time interval of 60 seconds or less.

159. The method and/or SPH material according to any of claims 130-158, wherein the SPH material in the Compressed State exhibits a Volume Swell Ratio of at least 20, at least 30, at least 40, at least 50, at least 60, at least 70 and/or at least 80.

160. The method and/or SPH material according to any of claims 130-159, wherein the SPH material in the Compressed State exhibits a Volume Swell Ratio that is at least 2 times, at least 3 times, at least 4 times and/or at least 5 times a Volume Swell Ratio of the SPH material in an Uncompressed State.

161. The method and/or SPH material according to any of claims 130-160, wherein the SPH material is an elastic material.

162. The method and/or SPH material according to any of claims 130-161, wherein the SPH material comprises one or more crevices formed therein.

163. An SPH material formed by a method according to any of claims 130 and 132-162.
